(19) United States
(12) Patent Application Publication
(10) Pub. No.: US 2024/0164801 A1
(43) Pub. Date: May 23, 2024

(54) RAPID CLOT REMOVAL USING ASPIRATION CATHETER WITH ASPIRATION GUIDED BY MONITORING ASPIRATION PERFORMANCE

(71) Applicant: MIVI Neuroscience, Inc., Eden Prairie, MN (US)

(72) Inventors: John Wainwright, Foothill Ranch, CA (US); Mayra Contreras, Santa Ana, CA (US)

(21) Appl. No.: 18/514,518

(22) Filed: Nov. 20, 2023

Related U.S. Application Data
(60) Provisional application No. 63/426,902, filed on Nov. 21, 2022.

(51) Int. Cl. A61B 17/22; A61M 1/00

(52) U.S. Cl. CPC A61B 17/22; A61M 1/85; A61B 2017/22041; A61B 2017/22079; A61B 2217/005

(57) ABSTRACT

Aspiration catheter systems are described that implement a procedure for aspirating a clot from a blood vessel using an aspiration catheter system, the method comprising:

initiating calibrated continuous aspiration;

evaluating aspiration measurements; and

adjusting application of aspiration based on aspiration measurements. In particular, pulsed aspiration can be applied selectively in response to flow measurements, such as values of the flow rate below a threshold value. The automated system can also use flow measurement to determine if the clot is presumably cleared as well as estimating the clot hardness. Effective aspiration can be applied with aspiration off periods shorter than the aspiration on periods. The corresponding aspiration system can comprise an automated system with a controller, an automatic valve, a flow rate sensor, a filter, and generally a pressure sensor. The catheter can be a full length aspiration catheter or a distal access aspiration catheter sharing the aspiration lumen with a guide catheter.

### CROSS REFERENCE TO RELATED APPLICATIONS

This application claims priority to copending provisional patent application 63/426,902 filed on Nov. 21, 2022 to Wainwright et al., entitled “Rapid Clot Removal Using Aspiration Catheter With Aspiration Guided by Monitoring Aspiration Performance,” incorporated herein by reference.

### FIELD OF THE INVENTION

The invention relates to aspiration catheter systems designed with fittings designed for efficient and safe operation of the aspiration treatment for use in bodily vessels with tortuous paths, such as cerebral arteries. In particular, the invention relates to suction catheter systems comprising a guide catheter and a suction extension slidably disposed within the guide catheter and to fittings allowing for efficient evaluation of the processing and real time adjustment of aspiration conditions, including use of pulsatile flow as appropriate.

### BACKGROUND OF THE INVENTION

Procedures in blood vessels of the brain are gaining use as an approach for ameliorating acute stroke events or other interventions in blood vessels in the brain. Blood vessels in the brain follow particularly tortuous paths which can increase the difficulty of reaching target locations in these vessels. Other vessels in a patient can also follow winding paths that increase the difficulty of reaching target locations.

Aspiration catheters have found use with respect to removal of clots from vessels. Furthermore, a significant reason for ischemic injury during percutaneous procedures can be generation of emboli that block smaller distal vessels. Aspiration catheters used alone or with embolic protection device can be effective to capture emboli generated during procedures. The delivery of effective devices to the small blood vessels of the brain to remove clots and/or to capture emboli remains challenging.

Ischemic strokes can be caused by clots within a cerebral artery. The clots block blood flow, and the blocked blood flow can deprive brain tissue of its blood supply. The clots can be thrombus that forms locally or an embolus that migrated from another location to the place of vessel obstruction. To reduce the effects of the cut off in blood supply to the tissue, time is an important factor. In particular, it is desirable to restore blood flow in as short of a period of time as possible. The cerebral artery system is a highly branched system of blood vessels connected to the interior carotid arteries. The cerebral arteries are also very circuitous. Medical treatment devices should be able to navigate along the circuitous route posed by the cerebral arteries for placement into the cerebral arteries.

### SUMMARY OF THE INVENTION

In a first aspect, the invention pertains to a method for aspirating a clot from a blood vessel using an aspiration catheter system, the method comprising:
initiating calibrated continuous aspiration;
evaluating aspiration measurements; and
adjusting application of aspiration based on aspiration measurements.

In a further aspect, the invention pertains to an aspiration thrombectomy system comprising an aspiration catheter assembly, fittings, a pump, a conduit, a flow meter, a filter, a first automatic valve, and a controller. Optionally, the aspiration catheter assembly further comprises a pressure sensor. The aspiration catheter assembly comprises a suction lumen extending from a proximal end with a connector, to a distal opening. The fittings comprise a branched manifold with a first branch comprising a hemostatic valve and a second branch comprising a connector, wherein the fittings are in fluid communication with the suction lumen of the aspiration catheter. The conduit is connected to the pump and to the connector of the second branch. The flow meter is connected to the conduit to measure flow rate to the pump. The filter is connected to the conduit to remove clots from the flow. The first automatic valve is configured to control flow between the fittings and the suction lumen. The controller is connected to the flow meter and the automatic valve and wherein the controller controls the valve based on measurements received from the flow meter and pulses the valve based on measured flow values.

In another aspect, the invention pertains to a method for aspirating a clot from a blood vessel using an aspiration catheter system, the method comprising the step of applying pulsed aspiration with alternating aspiration on periods separated by aspiration off periods, wherein the aspiration off periods are no more than half as long as the aspiration on periods and wherein the aspiration on periods are from about 0.25 second to about 25 seconds.

### BRIEF DESCRIPTION OF THE DRAWINGS

FIG. 1 is a flow chart of an overview of a procedure to clear a clot using an aspiration control system.

FIG. 2 is a fragmentary view of an embodiment of an aspiration system from a position in the neuro-vasculature to the proximal fittings.

FIG. 3A is a side view of an embodiment of an aspiration catheter.

FIG. 3B is a fragmentary side view of an aspiration catheter having a narrow diameter distal segment for placement in small vessels within the body.

FIG. 4 is a side view of a suction catheter system comprising a guide catheter with a suction extension with the guide catheter shown as transparent to allow visualization of structure within the guide catheter.

FIG. 5A is a side view of an embodiment of a guide catheter extending from a luer fitting to a distal tip.

FIG. 5B is a fragmentary sectional view of a portion of the guide catheter of FIG. 5A between points B-B in FIG. 5A with the cross section taken along a plane through the central axis of the catheter.

FIG. 5C is a fragmentary sectional view of a portion of the guide catheter of FIG. 5A between points C-C in FIG. 5A with the cross section taken along a plane through the central axis of the catheter.

FIG. 6 is a side view of an embodiment of a section extension.

FIG. 7 is a top view of the suction extension of FIG. 6 with some hidden structure shown with dashed lines.

FIG. 8 is a sectional side view of the suction extension of FIG. 6 taken along line 8-8 of FIG. 7.

FIG. 9 is a fragmentary sectional view taken along line 9-9 of FIG. 6.

FIG. 10 is a fragmentary sectional view taken along line 10-10 of FIG. 6.

FIG. 11 is a fragmentary sectional view of the catheter of FIG. 11 taken along an orthogonal view indicated by line 11-11 of FIG. 9.

FIG. 12 is a sectional end view of the catheter of FIG. 6 taken along line 12-12 of FIG. 8.

FIG. 13 is a fragmentary side view of an alternative embodiment of a suction extension with the expanded insert showing the attachment of a control wire to the proximal portion using a coiled end portion of the control wire.

FIG. 14 is a sectional view taken along line 14-14 of FIG. 13.

FIG. 15 is a fragmentary side view of an alternative embodiment of the proximal end of the control structure in which a handle is attached to the control structure and the end of the control structure is twisted to restrict the movement of the handle relative to its position on the control structure.

FIG. 16 is a fragmentary side view of a section of catheter wall with a sensor positioned exterior to the catheter wall.

FIG. 17 is a fragmentary sectional view of a section of catheter wall with a sensor positioned interior to the catheter wall.

FIG. 18 is a fragmentary side view of a suction tip with a bend.

FIG. 19 is a fragmentary side view of a suction tip with a bend and an angled opening.

FIG. 20 is a fragmentary side view of a suction tip with a gentle curve.

FIG. 21 is a sectional end view of a connecting section of a suction extension interfacing with engagement section of a guide catheter with a non-circular cross section.

FIG. 22 is a schematic depiction of a collection of medical devices that can be used together or in selected sub-combinations for selected percutaneous procedures in bodily vessels including a suction system as described herein.

FIG. 23 is a perspective view of a Y-branch manifold adapted for connection to an adaptive aspiration supply.

FIG. 24 is a perspective view of an embodiment of a valve in communication with a controller.

FIG. 25A is side view of a filter with a screen filter element in a compartment secured below a cap.

FIG. 25B is an exploded side view of the filter of FIG. 25A.

FIG. 25C is a cross-sectional view of the cap of the filter of FIG. 25A.

FIG. 25D is a cross-sectional view of the filter of FIG. 25A.

FIG. 25E is a side view of a filter with a screen filter element in a compartment secured below a cap with an electrical connection to a controller.

FIG. 25F is an exploded side view of the filter of FIG. 25E with electrodes encircling filter element and an electrical connecting exiting through a sealed hole in the filter body.

FIG. 25G is a cross-sectional view of the filter of 25E with electrodes encircling filter element and an electrical connecting exiting through a sealed hole in the filter body.

FIG. 26 is a side view of a pressure sensor with a display.

FIG. 27 is a side view of a pressure sensor.

FIG. 28 is cross-sectional view of a flow meter with a paddle wheel.

FIG. 29 is a cross-sectional view of a pressure sensor.

FIG. 30 is a schematic depiction of an embodiment of an adaptive aspiration supply.

FIG. 31 is a schematic depiction of a human patient with alternative access approaches for directing catheters into the blood vessels of the brain.

FIG. 32 is a view within a branched blood vessel section showing the delivery of medical devices along a guidewire from a guide catheter to a clot. Inserts show expanded views of two internal sections of the guide catheter.

FIG. 33 is a schematic view in a section of blood vessel of a suction system being used to remove a clot.

FIG. 34 is a schematic view in a section of blood vessel with a suction system positioned upstream from a clot and a fiber based filter deployed downstream from the clot.

FIG. 35 is a schematic view of the section of blood vessel of FIG. 49 with the fiber based filter being drawn toward the suction tip to draw the clot to the tip for facilitating removal of the clot.

FIG. 36 is a schematic view of a section of blood vessel with a suction system positioned upstream from a clot, a fiber based filter deployed downstream from the clot and another medical device positioned at the clot.

FIG. 37 is a schematic view of the section of blood vessel of FIG. 51 with the various medical devices being used in concert for the removal of the clot.

FIG. 38 depicts an integrated display with the display showing patient imaging and windows indicating pressure and flow.

FIG. 39 is a flow chart of a procedure to clear a clot using an aspiration control system.

FIG. 40 is a schematic view of an experimental system configured to allow for real time monitoring of a simulated aspiration thrombectomy.

FIG. 41 is a perspective view of a synthetic clot.

FIG. 42 is a closeup view of a flow sensor.

FIG. 43 is a side view of a distal portion of a catheter.

FIG. 44 is a side view of a synthetic clot in tubing.

FIG. 45 is a side view of a catheter positioned within the tubing of FIG. 44 at a position near the synthetic clot.

FIG. 46 is a chart depicting pressure and flow rate measurements for an exemplary trial.

FIGS. 47A-471 are charts depicting pressure and flow rate measurements for exemplary trials using a Q4 catheter.

FIGS. 48A-48L are charts depicting pressure and flow rate measurements for exemplary trials using a Zoom45 catheter.

FIGS. 49A-49D are charts depicting pressure and flow rate measurements for exemplary trials using a 4MAX catheter.

FIG. 50 is a comparison of results with a Q6 catheter and a Sophia6 catheter.

FIG. 51 is a set of charts depicting flow and pressure measurements where a valve is closed and reopened at selected increments.

FIG. 52 is a chart depicting results from trials with a simulated soft clot with aspiration pulsed.

FIG. 53 is a set of charts with representative pressure and flow measurements of medium hardness clots and hard clots plotted in superimposed format.

FIG. 54 is a box plot of catheter ingestion time during trials.

FIG. 55 is a box plot of system ingestion time during trials.

FIG. 56 is a plot of flow and pressure during a trial procedure.

FIG. 57 is a plot of flow and pressure during a trial procedure using a 5 mm clot.

FIG. 58 depicts an expanded portion of the plot of FIG. 57.

FIG. 59 depicts a plot of flow and pressure during a trial procedure.

FIG. 60 depicts an expanded portion of the plot of FIG. 59.

FIG. 61 is a box plot of ingestion times for a Q4 catheter comparing constant aspiration with a pulsed aspiration procedure.

FIG. 62 is a box plot of ingestion times for a Q5 catheter comparing constant aspiration with a pulsed aspiration procedure.

FIG. 63 is a plot of pressure as a function of time using a Q5 catheter for short times with soft clots.

FIG. 64 is a plot of pressure as a function of time using a Q5 catheter for short times with medium hardness clots.

FIG. 65 is a plot of pressure as a function of time using a full length aspiration catheter for short times with both soft and medium hardness clots.

### DETAILED DESCRIPTION OF THE INVENTION

Based on understanding gained from studies of model clot clearance, an understanding of flow and/or pressure measurements can be applied for the improvement of aspiration thrombectomy procedures to improve results and reduce potential negative impacts on the patients. In particular, it is possible to identify different stages of clot movement under the procedure, which can be monitored automatically, to more effectively apply aspiration while also shortening procedure times. Also, clot hardness can be estimated from the measurements (related to flow and/or involving external evaluation), and subsequent clot removal efforts can be modified based on the measurements. Automated control of the flow can effectuate more effective treatment and reduce risk to the patient. Flow movement through the system can be rapid, so manual response would generally be significantly slower than automatic response. In combination with sensor measurement, pulsed aspiration can be adopted to safely speed the clot removal. Processes are described for the real time evaluation of flow conditions to automatically implement pulsatile flow when flow measurement indicate the likely improved results resulting from the use of pulsatile flow. Pressure and flow measurements are used to evaluate clot hardness, and a determination of clot hardness can be used to select the most appropriate aspiration profile. By shortening treatment times and quickly ending the procedure after clot clearance, reduced stresses can be applied to the blood vessel without compromising clot removal efficacy.

As described below, the improved processes make use of aspiration catheter systems that provide the functionalities providing for the processes described herein. The aspiration processes described herein generally can be effectively used in various aspiration catheter systems, including various unitary catheter based aspiration systems. But the process improvements can be particularly advantageous for systems with an aspiration catheter or suction extension (distal access catheter) that is designed to insert with its proximal end into a guide catheter with its distal tip extending past the guide catheter. The aspiration procedure can be considered as involving the following stages: initiation of aspiration, clot migration under aspiration to the catheter tip, corking of the clot at the catheter opening, restrained movement of the clot through the catheter, clearance of the clot from the catheter with clot capture in a filter, and stopping aspiration. The aspiration system comprises catheters, fittings, valve(s), sensor(s), a pump, and control systems, which all interface with each other.

In embodiments of particular interest, the catheters generally comprise an aspiration catheter and a guide catheter, and improved clot removal is observed with catheter system involving the guide providing a portion of the aspiration lumen. Results presented below compare aspiration efficacy with an aspiration catheter system with a portion of the aspiration lumen within the guide catheter with commercial versions of unitary aspiration catheter systems. As used herein, fittings refer in this context to the connectors, tubing, and the like, that connects the catheter to the sensors, valve(s) and a pump and maintains a clean volume behind hemostatic valves and the like. Fittings are known in the art and in some form are used in basically all catheter procedures, although particular configurations are selected for the application. Sensor(s) generally comprises a pressure sensor and/or a flow sensor. Sensors can be integrated into the catheter itself, but experiments are presented with external sensors, and the procedures herein are based on sensor readings made in the fittings. Overall, the system generally comprises a plurality of valves with at least one automatic flow valve (such as a solenoid valve) along with one or more hemostatic valves, an isolation valve, and possibly (manual or automatic) valves to control or read flow direction. A control system generally comprises a processor with suitable memory and instructions, displays, and interfaces with one or more valves, one or more sensors and optionally the pump. Control boards designed for sensor operation can be adapted to control automatic valve operation.

Pulsed aspiration has been suggested to be an instrument to significantly improve efficacy of clot removal. For example, it was speculated the pulsed aspiration would apply stresses that would help break up clots to facilitate removal, see WO 2014/151209 to Grey et al., entitled “Dynamic Aspiration Methods and Systems,” incorporated herein by reference. In model studies, significantly improved clot clearance was observed with cyclic aspiration. Later studies suggested providing venting to result in back flow during part of the aspiration cycling, but this approach seems to offer risk of allowing the clot to travel upstream in the vessel possibly to locations where it no longer could be retrieved. See, U.S. Pat. No. 11,464,528 to Brady et al., entitled “Clot Retrieval System for Removing an Occlusive Clot From a Blood Vessel,” and U.S. Pat. No. 11,096,712 to Teigen et al., entitled “Aspiration Thrombectomy System and Methods for Thrombus Removal With Aspiration Catheter,” incorporated herein by reference. As described in U.S. Pat. No. 10,531,883 to DeVille et al., “Aspiration Thrombectomy System and Methods for Thrombus Removal With Aspiration Catheter,” and published U.S. patent application, the system is designed to control the venting to limit reverse flow from the distal end of the catheter. Nevertheless, venting creates potential risks and complications to control the flow.

Applicant has found that the catheter design is a significant factor in aspiration efficacy, but appropriate use of pulsed aspiration can further improve efficiency of clot clearance. The results presented herein demonstrate these results. In particular, flow and pressure measurements are used to perform real time evaluation of the clot clearance with the selected use of pulsing of the aspiration to facilitate clot clearance. Pulsed aspiration, which can be automatically controlled, can be focused on times during which the clot is corked to reduce risk of clot fragmentation or loss as well as to further speed the process. Safe and effective clot clearance can be effectively deployed, and the catheter system using sections of the guide catheter for forming the aspiration lumen is demonstrated to provide superior clot clearance relative to single catheter systems. Indiscriminate pulsing of the aspiration can slow the clot clearance.

Applicant has developed an aspiration system incorporating a flow meter along with a pressure sensor to evaluate the status of the flow. The flow measurements are complimentary and in some ways more diagnostic of the status in the catheter system and measurements of the pressure sensor. These measurements are examined in detail in bench studies discussed below. The initial incorporation of a flow meter into aspiration system was described in published U.S. patent application 2023/0029243 to Ogle (hereinafter the ′ 243 application), entitled “Suction Catheter Systems With Designs Allowing Improved Aspiration and Evaluation of Aspiration Condition,” incorporated herein by reference. The aspiration systems herein further comprise an automatic valve that provides for systematic evaluation of the flow status and control of the flow accordingly. Pulsed aspiration can be used when the flow status indicates desirability of pulsed flow.

The use of flow meters and/or pressure sensors to assist with aspiration thrombectomy is described more specifically in copending U.S. patent application Ser. No. 17/667,828 to Wainwright et al. (hereinafter the ′828 application), entitled “Suction Catheter Systems With Designs Allowing Improved Aspiration and Evaluation of Aspiration Condition,” incorporated herein by reference. As described in the ′828 application, one or more flow meter(s) and/or pressure sensor(s) can be independently placed in different locations, such as integral with a catheter, in the proximal fittings adjacent other components, or adjacent the pump.

The procedures described herein are designed to improve aspiration thrombectomy by automatic response based on sensor measurements. A flow shut off valve can cut aspiration to provide pulsed aspiration at appropriate times in the procedure. The flow shut-off valve can similarly stop aspiration automatically once the clot is safely captured and/or reduce aspiration pressures at the later stages of clot clearance, such that continued aspiration is not depleting blood flow in the vessel or potentially harming the vessel. The control system can be adapted to provide these functionalities based on sensor readings. With effective clot removal, pulsed aspiration may only be desired for a limited extent to improve clearance efficacy.

The procedure begins with assembly and preparation of the catheter system. The specific options for the catheter system are discussed in the following. For preparation, the components are filled with sterile saline or similar biocompatible fluid, such as those known in the art. The pump can be effectively used to prime the system with sterile fluid, such as by placing the catheter into the sterile fluid reservoir and running the pump to suck the fluid through the catheter components and fittings. During priming, the system can be checked for air leaks. Flow and/or pressure not corresponding to the standard values for the catheter system can indicate air leaks that can be eliminated prior to insertion into the patient. Once the system is primed and checked, it can be introduced into the patient using conventional introducers or the like to provide access into the vessel.

For access to cerebral vessels, access can be made through the femoral artery with the catheter tracked up the aorta and guided into the right or left carotid artery prior to reaching the heart. In alternative embodiments, the catheter can be introduced into an artery in the arm, through the brachiocephalic artery and then guided into the left or right carotid artery, or other reasonable access point. While the catheter can in principle be delivered directly into a carotid artery in the patient's neck, this is generally considered a less stable delivery configuration. The discussion herein focuses on the femoral artery access, but modifications for other access routes generally involve changes within the knowledge of persons of ordinary skill in the art based on the teachings herein. Additionally, if an on/off switch or valve is placed proximal to the pressure sensor, the pressure could measure the pulse pressure while navigating. Obtaining local pressure changes may be useful especially if IntraCranial Atherosclerotic Disease (ICAD) is present. Fidelity of the measurement would be further enhanced if the pressure sensor was at the catheter junction or near distal tip. This would operate much like an Arterial line (A-line) but provide local measurements. If the pressure sensor is further distal it would provide higher resolution/lower capacitance of pressure sensing.

The results herein confirm the excellent performance of Applicant's Q-Catheter™ design, which has an aspiration catheter (distal access catheter) positioned to use a section of the guide catheter for the aspiration lumen with a narrower tip extending past the guide catheter into smaller vessels. See also U.S. Pat. No. 10,716,915 to Ogle et al. (hereinafter the ′915 patent), entitled “Catheter Systems for Applying Effective Suction in Remote Vessels and Thrombectomy Procedures Facilitated by Catheter Systems,” incorporated herein by reference. These results are consistent with clinical evaluations. See Kobeisi et al., “Mechanical thrombectomy with Q catheter in stroke caused by primary and secondary distal and medium vessel occlusion,” Interventional Neuroradiology April 2023 (doi:10.1177/15910199231167915), incorporated herein by reference. The improved control features described herein should further improve clinical efficacy of the Q-catheter or other aspiration catheter designs.

Aspiration Control System

For the control processes described herein, the overall procedure involves preparing the system, initiating aspiration and evaluation of the status of the clot. With an efficient aspiration catheter, soft clots can be rapidly and effectively cleared. If the clot is exhibiting corking in the catheter, pulsed aspiration can be delivered. It has been found that aspiration pulses comprising short breaks in the aspiration can be very effective in resumption of clot movement into and through the catheter. Based on the evaluation of aspiration status, the hardness of the clot can be estimated, and the aspiration can be adjusted to improve clearing of medium hardness and hard clots. To improve the timing and reduce blood loss, the system response to sensor readings can be automated with oversight by a health care professional.

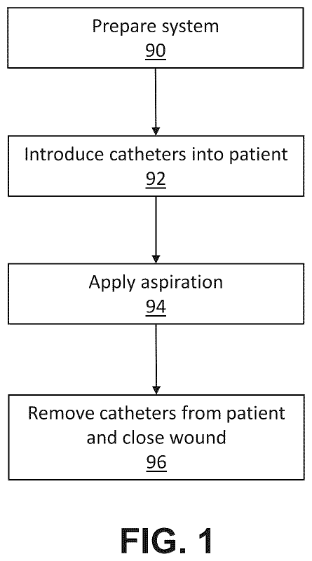

An overview of the procedure can be summarized with the following steps, although additional steps can be added before, after or intermittently as desired. See also FIG. 1.

1. Preparing the System (90)

2. Introducing the catheters into the patient and positioning for aspiration. (92)

3. Applying aspiration, which has the following stages: (94)

Aspiration Stages:
A. Initial clot engagement. This time period can be short.
B. Corked, at catheter tip and/or within catheter (not necessarily observed).
C. Uncorked moving—Presumably this free flow continues through the fitting until the clot reaches the filter and is removed from the flow. This time period can be short.
D. In filter and terminating aspiration. Since flow is fairly predictable at this stage and since any non-predictable flow can be measured, it seems that this stage can be determined accurately once any transient measurements are avoided.

4. Remove catheters from patient and close wound. (96)

With respect to preparing the system (90—FIG. 1), the flow meter can be zeroed following manufacturer instructions for the zeroing particular flow sensor being used. With respect to calibrating the flow and pressure sensors, this can be done each time the system is used, or it can mainly rely on look up tables for values with periodic resetting of the look up table to allow for changes of time or pump performance, replacement if basic components are not performing adequately, or simply occasional confirming of values.

Specifically, following system assembly, the catheter system can be primed with sterile biocompatible fluid, such as sterile saline. Once the system is fully primed, the system can be checked for air leaks using the pressure and/or flow sensors. In a properly functioning system, the freely flowing saline or the like provides a high flow limit and a low pressure limit with the pump on and valve(s) fully open. The measured values can be checked against manufacturer specified values, which can be programmed into the controller. Based on pressure, values measured can be compared with expected values. If below a certain first threshold, such as 95% of expected value, this would indicate that a check for air leaks should be performed. If measured pressures are below a second threshold, such as below 80% of expected, this would indicate either there is an air leak or other system malfunction, such as a malfunctioning pump. If under the first threshold but above the second threshold, a check for air leaks should be performed and fixed if found prior to continuing with the procedure. If under the second threshold, an air leak should be corrected or other system problems identified and fixed prior to proceeding, after confirming with a further check. Similarly, with flow measurements, a limit can be set for checking, such as ±20% of expected flow. If both values are used, these can be used to confirm each other to reach comfort that system is operating within acceptable parameters prior to continuing.

Since each system can vary from others somewhat based on specific layout of the system and pump performance, which likely varies somewhat over time. Values provided by the instructions for use (IFU, standard values) can provide peak operating values for flow and pressure. The check procedure can identify issues such as air leaks as well as more significant failures, such as a pump not performing adequately. Once a checked system is operating within acceptable parameters, the actual measured limits of flow and pressure for that specific system can be used as an alternative to standard values for the subsequent procedure evaluations. This procedure can allow for operation of the basic system using different catheter sizes as well as brands of components, such as a guide catheter.

The introduction of the catheter into the patient (92—FIG. 1) can rely on known percutaneous catheter procedures, which can involve, for example, an introducer, hemostatic components and suitable guidewires to access the treatment zone. Similarly, removal of the catheter from the patient 96 generally can comprise standard catheter procedures involving removal of components from the patient while avoiding risk of contamination. It can be desirable to check vessel flow using contrast dye and imaging prior to removal of the guide catheter and/or the aspiration catheter. Between introduction and removal of the catheters, dynamic aspiration procedure 94 can be performed, which is described in various embodiments in detail below.

In the improved procedures described herein, the performance of the aspiration stages can be described in the context of a dynamic aspiration procedure. Pressure and flow measurements provide information regarding the state of the clot removal process. The pump pressure generally provides a negative (gauge) pressure that can be evaluated when there is no flow, which provides the maximum negative pressure available for that pump. Ambient atmospheric pressure provides the pressure reference point. Pumps can be considered as referring to any suitable negative pressure device, but certain medical pumps are available that can be used for this function, such as available from MIVI Neuroscience, Inc. When flow is unrestricted as when the catheter is placed in a liquid pool, such as sterile saline, the basic catheter system has a corresponding flow rate and pressure that corresponds with one limit with open exposure to the pump.

Catheter system pressures and flow rates for 11 commercial aspiration catheters are provided in Table 1, where the catheters are placed into a liquid pool. Pressures range from about −18 inches of mercury (inHg)) to about −27 inHg. Flow rates range from about 1 milliliter per second (ml/s) to about 7 ml/s. The flow rates are grouped approximately by outer diameters at the distal end of the catheters. A higher flow rate correlates with a lower pressure since the flow results in a reduced pressure and vice versa. These two extremes provide a basis for evaluating aspiration status.

TABLE 1

| | | Ave. | Outer |
|---|---|---|---|
| Catheter | Ave. Flow | Pressure | Diameter |
| (TMs) | (ml/sec) | (inHg) | (mm) |
| Q6^{a} | 7.0 | −18.2 | 2.13 |
| SOFIA6^{b} | 5.3 | −21.6 | 2.1 |
| Q5^{a} | 5.8 | −19.6 | 1.83 |
| SOFIA5^{b} | 3.0 | −25.2 | 1.7 |
| ZOOM55^{c} | 4.3 | −22.8 | |
| Q4^{a} | 4.8 | −23.0 | 1.4 |
| 4MAX^{d} | 2.6 | −25.4 | 1.42 |
| ZOOM45^{c} | 3 | −25.0 | 1.52 |
| Q3^{a} | 3.3 | −24.6 | 1.22 |
| 3MAX^{d} | 1.2 | −27.2 | 1.27 |
| ZOOM35^{c} | 1.3 | −27.3 | 1.3 |

^{a}Q6 ™, Q5 ™, Q4 ™, Q3 ™ are catheter systems from MIVI NeuroScience.
^{b}SOFIA6 ™ and SOFIA5 ™ are catheters from Microvention.
^{c}ZOOM55 ™, ZOOM45 ™, and ZOOM35 ™ are catheters from Imperative Care.
^{d}4MAX ™ and 3MAX ™ are catheters from Penumbra.

Pressure and/or flow measurements between the unrestrained flow and no flow extremes provide information on the status of the clot movement that can guide the procedure. At one extreme, if the clot is “corked” in the catheter, the pressure drops (becomes more negative) and flow drops relative to the unconstrained flow, which can drop to essentially zero flow. “Corked” can refer to the clot engaged at the catheter tip without free movement into the catheter, or to clots that remain within the catheter lumen for more than 30 seconds. Thus, corking can be related to insertion into the tip, or it can involve clots wedged in the catheter lumen and not effectively moving. A clot freely moving within the catheter should be cleared in a few seconds. If fully corked and non-moving, the flow would be zero and the pressure would be at the pump negative pressure limit. If the clot is fully corked, i.e., no flow around the clot within the catheter, but is moving, the flow could be low and the pressure near the pump limit. In this embodiment, the flow would be close to the volume swept by the moving clot, and since the unconstrained flow can clear the catheter and fittings fully in seconds, depending on the flow rate. If the clot is not fully corked, intermediate values of flow and pressure would be obtained, and it may be difficult to directly evaluate the rate of clot movement. It can be difficult to tell whether or not the clot is corked and essentially stationary, or moving somewhat. But it is clear that flow is passing around the clot and not fully corked if the flow and pressure are intermediate and if the clot is not cleared into the filter in the expected time based on the flow volume indicating a clearing of the catheter volume.

It is desirable to have a larger free flow rate since this suggests a stronger aspiration at the catheter tip. Once the clot is cleared, it is desirable to end the aspiration to avoid continuing to pull blood volume out of the vessel without any need. With higher flow catheters, the results indicate that the clot clearance time can be reduced significantly, so the aspiration time, may be relatively short to achieve good clot clearance. As described herein, a risk of injury to the vessel can be mitigated through the control system described herein.

Some objectives of using the sensor measurement in controlling the aspiration relates to more effective use of the aspiration as well as reducing or avoiding any potential injury to the vessel. To effectuate these improvements, the flow system generally comprises a valve with automatic control. Suitable valves are described below, but generally the valves can be actuated (or de-actuated) to fully cutoff aspiration, or with some valves the amount of aspiration can be controlled with partial closure. In some embodiments, it is desirable to pulse the aspiration to facilitate getting the clot uncorked in the catheter. Based on this objective, it is correspondingly desirable to not pulse the aspiration when the clot is not corked to avoid slowing completion of clot removal. Alternatively, a system like the Phillips QuickClear™ could be used where the vacuum pump can provide a baseline and then the vacuum syringe can be used by a health care provider when indicated by software so that pulsatile aspiration can be applied. Alternatively, the syringe can provide the base line vacuum pressure and then the pump with or without a valve could be cycled to provide the pulsatile pressure. These configurations would ensure that there was always some level of vacuum and prevent clot lose.

Also, it can be desirable to terminate aspiration once the clot is fully removed and captured in a filter and out of the flow. Similarly, with a partially closed valve, the flow can be reduced incrementally or gradually as the clot is nearing capture to avoid a flow spike as the clot is captured. High flow, unrestricted aspiration can result in vessel collapse and potential injury to the vessel once the clot is not inhibiting the full force of the aspiration from reaching the vessel. So once the clot is captured, it is desirable to terminate the aspiration, which can be accomplished by closing the valve connecting the catheter to the pump. Similarly, with an appropriate valve design, flow can be reduced (stepwise or gradually) at the last stages of clot capture so that the amount of negative pressure reaching the vessel at the time of clot capture can be reduced.

Following a discussion of the components of the system, these procedural steps are discussed in more detail, and lab bench results are presented to illuminate operation of these steps in a controlled environment. For exemplified versions described herein, a disposable flow meter is used that is placed close to the fittings adjacent to entry points into the patient. A pressure sensor is placed adjacent to the flow meter. Lower cost flow meters based on thermal sensing are available from Innovative Sensor Technology (such as OOL, thermal mass flow sensor). In some embodiments, a control board interfaced with the flow meter can be used to automate the process flow, although alternative separate processors can be similarly used.

An aspiration catheter with one or more components and a pump can be considered the respective ends of the aspiration system. Fittings generally attach to a hub at the proximal end of a catheter to provide for a sealed connection to the interior of the catheter. The fittings provide for a sealed connection to the pump and generally comprise various branches that can provide for introducing and/or removing fluids as well as optionally providing for introduction of ancillary medical devices for delivery into the patient. If the fittings are considered to include the components assembled between the catheter and the pump, the fittings generally also comprise one or more valves, tubes, and the sensors are integrated into the fittings at selected locations. In alternative terminology, the catheter fittings can be limited to components that connect with specific connectors, such as Luer connectors, hemostatic values, Tuohy-Borst adapters, and the like, and transitioning to tubing, such as high pressure tubing, to connect the fittings to the pump can involve different styles of connectors, but regardless of the terminology, the structures are clear. Tubing connectors are known in the art with different styles than Luer connectors and the like that are standardized for leak free connections for hypodermic or intravascular applications.

To provide desired control over the flow, flow to the pump can be controlled using one or more valves, such as an electronically controlled valve. In some aspiration systems, pulsing can be performed through turning the pump on and off. The results herein suggest the desirability of using a value to control flow. In some embodiments, the valve can be a solenoid valve mounted external to the tubing with closing of the valve resulting in a pinching closed of the tubing. Other valves, such as diaphragm valves or mechanical leaflet valves can be used alternatively, which may or may not be pinch valves in isolation from the flow. Commercial valves can be adapted for connecting to the aspiration tubing. As described in the ′828 application, an appropriate controller can operate the valve automatically in response to sensor readings and/or manually. As described herein, appropriate algorithms are described to effectuate such control. A commercial solenoid valve for mounting on tubing is available from Cole-Palmer® under the Masterflex series of two-way solenoid-pinch valves or electronic pinch valves from Clippard (Clippard Instrument Laboratories, Inc.). A proportional solenoid pinch valve is available from IMI Norgren® under the Acro 900 series.

A valve can be placed at reasonable locations within the fittings and/or tubing. Transient sensor readings may be somewhat sensitive to the location of the valve upon opening and closing, but transient readings should be sufficiently short in time as to not alter basic procedural functions. A plurality of pressure sensors can be desirable to measure pressures on opposite sides of the valve, which can be significantly different due to the relative location of the pump. In some embodiments, a plurality of valves can be used to accommodate and possibly take advantage of transient flows. For example, an electrically actuatable valve can be placed close to the catheter distal to any sensors and to the filter, while a second valve can be placed closer to the pump relative to the filter and possibly sensors. While any of the valves can be proportional valves that can partially close, placing a proportional valve proximal to the filter and toward the pump can avoid catching the clot on a partially closed valve since the clot should be removed by the filter prior to reaching the valve. In some embodiments, having a valve distal to the sensors allows for closing the valve initially with the pump on to set baseline sensor readings at the start of the procedure followed by automatic opening of the valve to initiate aspiration from the catheter.

A filter can be used to remove the clot from the flow once it is removed from the catheter(s). This has the advantages of providing possible visual confirmation that the clot has been captured, although confirmation with sensor readings can help rule out clot fragmentation, and shortening the procedure since a significant length of tubing can be used between the catheter and the pump to help ensure maintenance of sterile conditions. Filter designs can reduce impact of the filter on the flow and safely remove the clot form the flow such that the removed clot does not impact ongoing flow significantly if at all.

While pressure and/or flow sensors can be placed within the catheter and potentially deployed in the patent, having sensors in the fittings or tubing can provide desirable information on clot capture. While pressure sensors and flow sensors can provide complementary information, one or the other can be used alone. In the examples below, the use of the complementary information is described. The sensor readings can be referenced to the nominal maximum and minimum baselines, although keeping in mind that transient measurements can exceed the baseline values. As indicated below, correlations can be made between the sensor limits obtained with saline and equivalent flow and pressure readings with blood, which can be mimicked using aqueous glycerin 40%. The controller can be programmed with a look up table or other correlation mechanism, such as fitted equations, to perform the conversion at the start of the procedure, and the converted estimates of the flow and pressure limits can be used to control the procedure.

For the flow during prepping of the system for a procedure, the range is set by zero flow and steady state flow of saline (converted for blood if desired) through the unconstrained catheter, which is the effective 100% flow value, discounting transients or other anomalous circumstances. Correspondingly, for the pressure, during prepping of the system for a procedure, the greatest negative pressure is the value pumping on the system with no flow, and the smallest negative pressure generally is the value with unconstrained saline flow (converted for blood if desired) through the system. So if the clot is completely corked or if the valve is completely closed, no flow is expected, and the pressure can reaches its greatest negative pressure, although transients change these expectations. Once the clot is captured in the filter, the flow becomes unrestricted, and the flow can reach the corresponding 100% flow value and the pressure can reach its limit of smallest negative pressure, which is then the highest pressure with the pump on relative to the pressure sensor.

Once the clot is cleared, it is desirable to stop or significantly reduce flow since there is no advantage in continuing to aspirate blood from the vessel. Clot clearance is indicated by the flow going to 100%±a selected range and/or the pressure going to smaller negative pressure limit with a±selected range. Similarly, a sensor in the filter can indicate the collection of material. Confirming multiple of these factors can provide comfort that the clot is cleared and, and in particular, likely fully cleared. As described in the following, the sensor readings can also be used to control pulsed aspiration. If desired, the sensor readings can also be used for partial valve closure, which can be used to moderate amount of aspiration from the vessel. Some specific protocols are described further below. The general principles can be discussed generally in the context of particular uses of the sensor measurements.

For the improved control of the application of pulsed aspiration, after prepping the system, aspiration is started and the status of the clot is checked using flow and/or pressure. The hardness of the clot as well as the likely movement of the clot can be determined in the first few seconds such that an aspiration strategy can be confirmed for the next portion of the process. In the procedure above, the flow rate is used as a flow based measure to estimate clot hardness and its corresponding response to aspiration. It has also been observed in bench testing that a pressure pulse is observed for softer clots at the start of aspiration, generally within the first second or half a second. A pressure pulse of at least about 10% or about 20% of the pump pressure within the first 0.5 seconds of aspiration can provide an additional or alternative estimate of clot softness to direct pulsed aspiration. In some embodiments, information on clot hardness is input based on separate evaluations of the clot such as using imaging techniques. For example, CT imaging can detect calcium in the clot suggesting a harder clot or red blood cells in the clot suggesting a softer clot. See Patel et al., entitled “Increased Perviousness on CT for Acute Ischemic Stroke is Associated with Fibrin/Platelet-Rich Clots,” Am J Neuroradiol 42:57-64 Jan. 2021, and Niesten et al., entitled “Relationship between thrombus attenuation and different stroke subtypes,” Neuroradiology volume 55, pages 1071-1079 (2013), both of which are incorporated herein by reference. The degree of flow through the clot may also give relevant information. As described further below, information on clot hardness can be input into the controller to help direct the procedure. Further tracking of the process can continue with adjustments as appropriate.

Aspiration Catheter Systems

Aspiration catheter systems generally comprise a one or two component catheter, a hub, potentially control structures and other ancillary components. With respect to aspiration catheters, the designs tested in the Examples fall into two design groups. In a two component embodiment, a short catheter design is intended for placement into a guide catheter with the aspiration tip extending from the distal tip of the guide catheter into the blood vessel and a control structure attached to the catheter and exiting from the patient into the fittings. The aspiration lumen then extends through the aspiration catheter and through a portion of the guide catheter. Such a design is described further below and in U.S. Pat. No. 10,478,535B to Ogle (hereinafter the ′535 patent), entitled “Suction Catheter Systems for Applying Effective Aspiration in Remote Vessels, Especially Cerebral Arteries,” incorporated herein by reference. These embodiments of aspiration catheter systems are described further below. Alternatively, an aspiration catheter can extend the entire length of the aspiration lumen within the patient with the proximal end of the catheter exiting the patient and connected to the fittings. With the full-length aspiration catheters, a guide catheter is generally still used, and the aspiration catheter usually exits through a hemostatic valve or the like to connect to fittings. The full-length aspiration catheters can have a smaller diameter section near its distal end to provide access into smaller vessels, as described in U.S. Pat. No. 9,662,129B to Aldonic et al., entitled “Aspiration Catheters for Thrombus Removal,” incorporated herein by reference. For the exemplified aspiration catheters, Applicant's Q series catheters, based on the technology of the ′535 patent) have the short design using a portion of the guide catheter for the aspiration lumen, and the other catheters are full-length catheter designs. As demonstrated in the Examples below, the Q series catheters are able to provide rapid clot clearance in bench simulations.

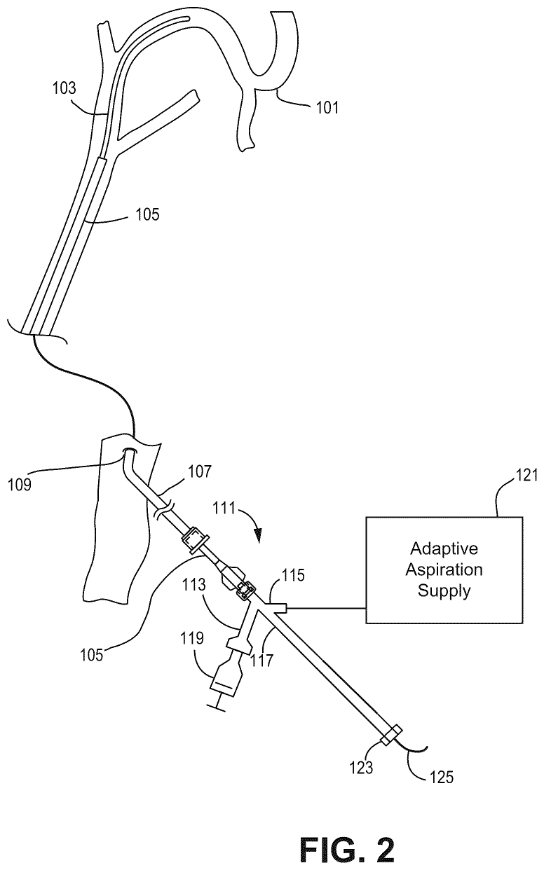

The general apparatus structure is shown schematically in FIG. 2. Details regarding specific embodiments and corresponding components can be found in the discussion below. With respect to FIG. 2, a fragmentary view of an embodiment of the aspiration system is depicted with elements of the catheters inserted into the patient. Details of the catheter are presented below. A distal portion of the aspiration system is shown in the neuro-vasculature 101 in which a aspiration catheter (distal access catheter design) 103 is extending from a guide catheter 105. A proximal portion of the aspiration system shows guide catheter 105 extending proximally from an optional introducer 107, which extends from the patient entry point 109. As shown in FIG. 2, proximal fittings 111 of the aspiration system extending proximally from guide catheter 105. In embodiments, proximal fittings 111 have various branches providing desired functionalities as described in the several embodiments presented herein. In embodiments, branches 113, 115, 117 may be multiple manifolds in various configurations such as a three branched manifold or two manifolds connected in series. In embodiments, first branch 113 is distal to second branch 115. In embodiments, second branch 115 is distal to third branch 117. In this particular embodiment, a first branch 113 may be connected to fluid source 119, which can be configured to delivery contrast agent, medication, a pressurized fluid or the like.

As depicted in FIG. 2, second branch 115 is connected to adaptive aspiration supply 121. Adaptive aspiration supply 121 can comprise a negative pressure source, a flow sensor, an automatic valve, a controller and optionally a pressure sensor. Specific embodiments and configurations are described below. Adaptive aspiration supply 121 provides adaptation of the aspiration that can provided the functionality for the specific aspiration procedures described herein. Third branch 117 can comprise a hemostatic valve 123 that allows for delivery of additional medical tools into the clean plenum of the catheter lumen as well as allowing exit and control of a control structure 125 of an aspiration catheter inside the guide catheter and/or a guidewire or the like. Particular configurations of proximal fittings 111 and can be assembled from a wide range of commercial fitting components or made to custom specifications.

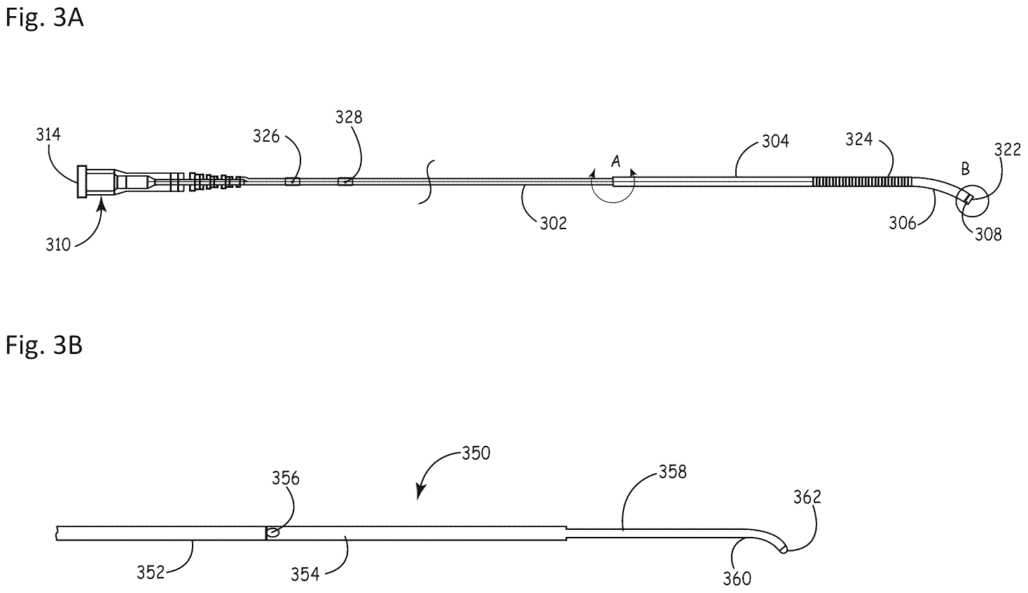

While the improved processing is relevant to both types of catheters, Applicant's primary interest and most of the experimental results relate to the Q-catheter design (distal access catheter), which does exhibit superior performance. Representative embodiments of a full-length aspiration catheter are found in FIGS. 3A and 3B. This embodiment is based on a rapid exchange design, but an over-the-wire counterpart just lacks the rapid exchange port and a rapid exchange segment just merges into a portion of the catheter tube. Referring to FIG. 3A, in a particular embodiment aspiration catheter 101 comprises a hollow tube 302, a rapid exchange segment 304 with an optional bent tip 306 and with a distal aspiration port 322 and a hub 310 for attachment to a suction device, which is shown as a hub with a female Luer fitting. In some embodiments, a single lumen extends from hub 310 through tube 302, through rapid exchange segment 304 to distal aspiration port 322. While it can be desirable for tube 302 to have a single lumen, in alternative embodiments tube 302 can have a plurality of lumen, such as two, three or more lumen, in which the lumen may or may not have different functions in the device. Hub 310 can comprises a handle and a connection port 314.

For rapid exchange embodiments, a guidewire port 121 is located at the position at which rapid exchange segment 304 joins tube 302. Rapid exchange segment 304 may or may not have a larger average diameter than tube 302. FIG. 3A show a first radiopaque band 308 positioned at the distal tip of the catheter, and a second radiopaque band 125 positioned at a selected position on rapid exchange segment 304. Visual marker bands 127, 129 are also shown.

Referring to FIG. 3B, aspiration catheter 350 for accessing smaller vessels comprises a tube 352, a rapid exchange segment 354, an optional rapid exchange port 356 at the position at which rapid exchange segment joins tube 352 and a reduced diameter distal segment 358 with an average diameter smaller relative to the average diameter of the tube with a curved distal tip 360 having a radiopaque marker band 362. While the proximal end of catheter 350 is not shown in FIG. 3B, catheter 350 can have a hub for connection to fittings similar to that shown in FIG. 3A.

The aspiration catheter can have a constant inner and/or a constant outer diameter along its length, but for either the distal access catheter or full-length catheter embodiments, a distal section can have a narrower configuration, which can be for both the inner and outer diameters. A narrower, smaller diameter, distal section can allow for access into smaller vessels. The narrow distal section can provide good aspiration compared with embodiments having the narrower diameter along its entire length. Having a narrow tip though raises concerns about getting the clot into the catheter, which suggests maintaining a larger diameter. Results are suggesting that having good aspiration is an important feature for clot removal and that clots are generally conformable for clearance. This is observed in the results presented herein, so the application of strong aspiration is desirable.

In some improved embodiments, the aspiration catheter can comprise a lubricious coating along the inner diameter. While the Q series catheters generally have good clot clearance once the clot is within the catheter lumen, a further speeding of the clot passage to the filter/trap and cleared form the flow can help to shorten the procedure. Application of coatings along the inner diameter can be provided to facilitate clot clearance.

To allow for a larger opening without compromising smaller diameters to provide access to smaller vessels, catheters with expandable distal tips have been proposed. Some designs to expand the inner diameter at the distal tip to the full diameter of the proximal portion are described in U.S. Pat. No. 7,309,334 to von Hoffmann, entitled “Intracranial Aspiration Catheter,” incorporated herein by reference. Catheter designs that allow for even greater distal tip expansion have been described in published PCT application 2016/113047 to Ribδ Jacobi, et al., entitled “Thrombectomy Device and System for Extraction of Vascular Thrombi from a Blood Vessel,” incorporated herein by reference. However, to form an actuatable tip involves significant design complexities and challenges to maintain sufficient flexibility of the tip with the extra design complexities.

The results herein indicate that good suction can be effective for removing clots through compression into the tip. In some situations, a significant amount of the ingestion time is entering the tip. This suggests that a modest bowing out at the tip may have significant advantages in using the suction to more directly deploy slight compressive forces to help guide the clot into the tip. Small bowing out at the tip can be implemented without significant structural changes that could impact delivery and flexibility. In some embodiments, to form such structures, a polymer body of a catheter with a thermoplastic composition can be softened at the tip and put over a mandrel of the like to gently form a funnel structure. Metal reinforcing wire can be reflowed into the polymer after the shape of the polymer is set, and this can be performed while the polymer tube remains on the mandrel. An embodiment of an aspiration catheter comprising a laser cut hypotube incorporated into the catheter structure can also conveniently adopt a slight bowing out at the tip, as described in copending U.S. provisional patent application 63/432,874 to Wainwright et al., entitled “Catheters With Laser Cut Hypotube Scaffold for Flow Under Different Pressure Differentials,” incorporated herein by reference. Potential embodiments are discussed further below.

A reasonable shape of the slight funnel can be selected as desired, and an approximately straight wall can apply a slight force along the length of the funnel. The inner diameter as a function of position can monotonically increase from the start of the outward expansion to the tip, which can take place at the end of a constant diameter segment. The diameter (inner and outer diameters) at the distal tip can be from about 0.5% to about 20% larger than the corresponding diameter at the adjacent constant diameter segment, and the length of the funnel can be, for example, from about 0.5 mm to about 1 cm. A person of ordinary skill in the art will recognize that additional ranges of dimensions and relative dimensions within the explicit ranges above are contemplated and are within the present disclosure.

With respect to bench trials described below, most results are obtained with soft clots, but some results are presented with medium hardness clots and hard clots. The medium and hard clots may damage the catheter tip under particularly challenging conditions further suggesting the slight bowing out described above.

Also, these results suggest the desirability of determining the hardness of the clot. If evaluated at or near the start of the procedure, the aspiration thrombectomy procedure can optionally be adjusted according to clot properties. For example, a fiber optic pressure sensor (such as Pressurewire™ X (Abbott) can be inserted a set distance into the clot and the pressure measured. The pressure reading should correlate with the clot hardness. Other biomedical photonics based measurements of the modulus or other property can be used. Alternatively or additionally, ultrasound or tomographic imaging can be used to look at the deflection of the clot after insertion of a wire, to evaluate the clot character. The use of imaging to measure strain in heart muscles using imaging is described further in Johnson et al., entitled “Practical Tips and Tricks in Measuring Strain, Strain Rate and Twist for the Left and Right Ventricles,” Echoresearch and Practice 6:3 R87-R98 (2019), incorporated herein by reference.

The suction catheter system is generally appropriately sterilized, such as with e-beam or gas sterilization. The suction catheter system components can be packaged together or separately in a sealed package, such as plastic packages known in the art. The package will be appropriately labeled, generally according to FDA or other regulatory agency regulations. The suction catheter system can be packaged with other components, such as a guidewire, filter device, and/or other medical device(s). The packaged system generally is sold with detailed instructions for use according to regulatory requirements.

Next, a detailed presentation of a Q-Catheter device and associated system is described. The catheter system is described in detail using a guide catheter and a distal access aspiration catheter for placement through the guide catheter and extending in a distal direction.

Aspiration Catheter Systems with Two Component Aspiration Lumen

The following aspiration catheter system can take advantage of good suction available with a suction lumen having a larger proximal section and a narrower diameter suction extension through a small aspiration catheter that uses the guide catheter lumen as a proximal suction lumen. These aspiration catheter systems provide an alternative to the full length aspiration catheters discussed above. A laterally slidable suction extension or distal access aspiration catheter extends from a proximal section located within the guide catheter lumen, and the small aspiration catheter can have a smaller distal diameter to provide access to narrow vessels while providing for delivery of other treatment structures and/or embolic protection structures as well as for a desirable level of suction for the removal of debris from the vessel. Herein, suction extension and distal access aspiration catheter terminology are used interchangeably. A control wire or other control structure can be attached to the suction extension to control sliding for providing selective lateral placement of the suction extension relative to a fixed guide catheter and a target treatment location. In some embodiments, the distal access aspiration catheter comprises a connecting section that interfaces with the guide catheter lumen with a non-cylindrical cross section to provide for contact at two parts along the circumference. This non-cylindrical interface can substantially block flow between the exterior of the proximal portion of the suction extension and proximal locations in the interior of the guide catheter while allowing relatively easy sliding of the suction extension relative to the guide catheter. Alternative connection section designs are mentioned below. The fitting, sensors and aspiration control and delivery components in this section can be equally applied for systems adopting full length catheters.

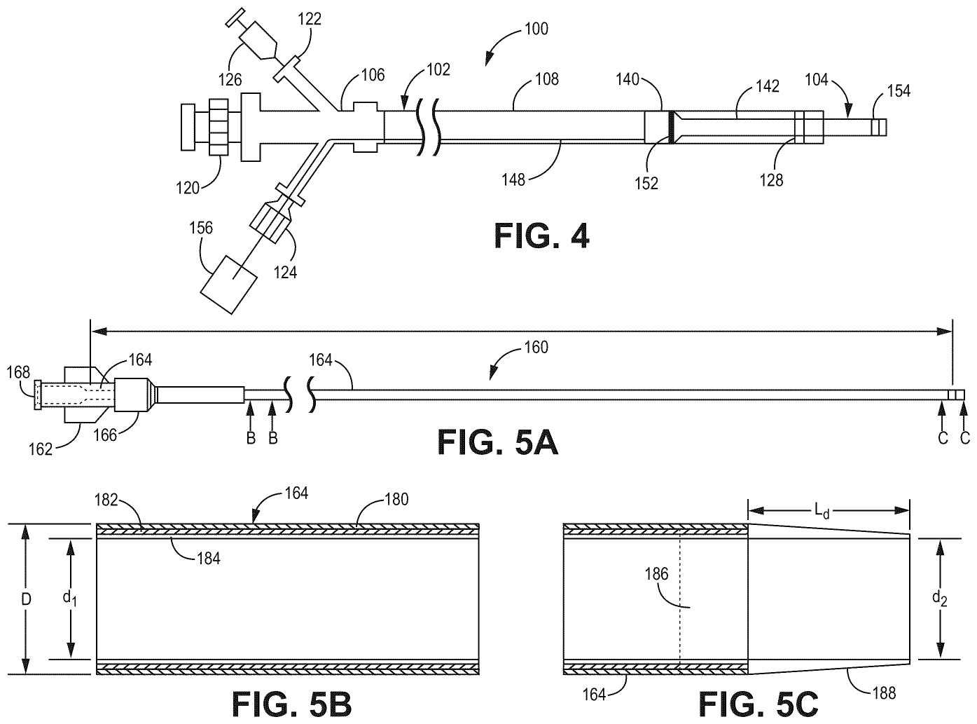

Referring to FIG. 4, suction system 100 comprises a suction adapted guide catheter 102 and a suction extension 104. The suction adapted guide catheter 102 comprises proximal section 106 and tubular shaft 108. Proximal section 106 generally is suitable for use also as a handle and generally can comprise proximal fittings 120, a suction port 122 and an optional control wire port 124, as well as possibly other additional ports and/or fittings to provide desired functionality and access, in which all such ports and fittings can be arranged in a branch configuration or other suitable configuration. In general, proximal fittings 120 can comprise a suitable hemostatic valve, luer connector or the like to provide for entry of a guidewire and/or structures delivered over the guidewire into the guide catheter lumen, such as alternative treatment structures and/or embolic protection devices.

Proximal fittings 120 can be configured with various components to facilitate handling or exchange of small aspiration catheter 104. See U.S. Pat. No. 11,234,723 to Ogle (hereinafter the ′723 patent, entitled “Suction Catheter Systems for Applying Effective Aspiration in Remote Vessels Especially Cerebral Arteries,” and 11,617,865 to Ogle (hereinafter the ′865 patent), entitled “Suction Catheter Systems With Designs Allowing Rapid Clearing of Clots,” both of which are incorporated herein by reference. In improved embodiments described herein, proximal fittings 120 are connected to adaptive aspiration supply 121 of FIG. 2. While desired features of fittings at the proximal end of the suction system 100 can be integral with proximal fitting 120, design flexibility can be achieved through embodiments of proximal fitting 120 comprising a connector, such as a Tuohy-Borst connector, and connection of fittings providing other desired features as described further below. For use with suction system 100, suitable embolic protection devices can be mounted on a guidewire, and/or other treatment structures can be used. Suitable treatment structures are described further below and can include, for example, fiber-based filters, stents, stent retrievers, atherectomy devices or the like.

In general, tubular shaft 108 can have an approximately constant diameter along its length, or some guide catheters can have sections with different diameters, generally with a smaller diameter section distal to a larger diameter section. In some embodiments described herein, a significant of the length of the tubular shaft has a constant diameter to make desired contact with a connecting section of the suction extension, which can be called an engagement section of the tubular shaft designed to engage the suction extension in a configuration suitable for the delivery of suction to a patient. Portions of the tubular shaft proximal to the engagement section can have a larger inner diameter and generally larger outer diameter relative to the engagement section. While a conventional guide catheter can be used in some embodiments for the suction catheter system, a specific design is described in detail below. A distal tubular portion of the tubular shaft can have a slightly narrower inner diameter, a tab or other structure to retain a portion of suction extension 104 within tubular shaft 108. Tubular shaft 108 can have one or more radiopaque marker bands to facilitate positioning of the tubular shaft within the patient as well as positioning the connecting section of the suction extension within the guide catheter lumen, and FIG. 4 shows a marker band 128 near the distal end of tubular shaft 108, although alternative positions can be used as desired. Tubular shaft 108 can have coatings on the inner surface and/or the outer surface or portions thereof.

Suction extension (distal access aspiration catheter) 104 generally comprises a connecting section 140, tubular extension 142, and control structure 148, such as a control wire. All or a part of connecting section 140 can be configured to remain within the lumen of guide catheter 102. As shown in FIG. 4, connecting section 140 can comprise a radiopaque marker band 152, although connecting section may not have a marker band in some embodiments and in other embodiments can comprise a plurality of marker bands, and tubular extension 142 is shown with radiopaque marker band 154 near the distal tip of tubular extension 142, although again tubular extension 142 can comprise a plurality of radiopaque marker bands if desired. Control structure 148 can be a control wire or the like that connects with proximal portion 140 and in the assembled device extends exterior to the catheter, such as exiting through control wire port 124 or proximal fitting 120. Control structure 148 can be used to control positioning of connecting section 140 within the lumen of shaft 106. Control structure 148 can comprise a control tool 156, such as a handle, slide or other the like that can anchor a control wire or other connecting element to facilitate movement of the control wire. In some embodiments, alternative structures such as a plurality of wires or cylindrical wire assembly can connect the proximal portion to the proximal end of the suction catheter system to provide a desired level of control with respect to positioning the proximal section.

As noted above, the connecting section of suction extension engages the inner lumen of the guide catheter with an appropriate interface to reduce or eliminate flow of blood between the connecting section of the suction extension while allowing for the user to translate the suction extension relative to the guide catheter to position the tip of the tubular extension. A desirable design with a connecting section of the suction extension having a non-circular cross section has been found to particularly meet these criteria. With material selection as described herein, a very small average clearance can also be used between the connecting section of the suction extension and the interior of the guide catheter. When assembled, the inner lumen of the guide catheter can contact the connecting section of the suction extension at two locations around the circumference, which can provide partial rounding the cross section of the connecting section. This two location contact configuration provides desirable confinement of the flow while allowing for sliding of the suction extension by the user with appropriate ease.

The non-circular cross section of the connecting section (or a portion thereof) of the suction extension generally can be roughly oval in shape. As described below, the oval shape can be generated through the attachment of a wire control structure to the proximal section, although other structural features can be used to introduce the oval shape, such as with approximately one axis of symmetry or two axes of symmetry, although the oval can be asymmetric. Generally, the oval cross section can be partially characterized by a major axis, e.g., the longer dimension along an axis of symmetry, and a minor axis, e.g., the longest line segment connecting the circumference perpendicular to the major axis. While the specification of the major axis and the minor axis does not fully specify the oval since the specific shape is not specified, the major and minor axes can provide significant information regarding the dimensions and relative shape of the oval, especially since the shapes are generally not far out of a circular shape. Also, an average clearance can be defined using the largest value of the circumference (C) of the oval cross section and converting to an equivalent circle to define an approximate average diameter (Dₐ=C/π).

An embodiment of a guide catheter is shown in FIGS. 5A-5C. Referring to FIG. 5A, guide catheter 160 comprises a connector fitted hub 162 with a portion of a Tuohy-Borst connector, luer connector or the like, shaft 164 and strain relief support 166. In this embodiment, the proximal end of shaft 164 passes through strain relief support 166 to connector fitted hub 162, and the components can be secured together with adhesive. Also, female connector 168 is located at the proximal end of connector fitted hub 162 for connection to a male connector fitting on a proximal fitting, such as a branched connector, which may have a rotating hemostatic valve with one or more branches.

A sectional view of a portion of shaft 164 near the proximal end is shown in FIG. 5B. Referring to the embodiment of FIG. 5B, shaft 164 comprises a polymer tube 180 with an embedded stainless steel wire braid 182 and a lubricious liner 184, e.g., polytetrafluoroethylene (PTFE) or other fluoropolymer. FIG. 5C shows the distal end of shaft 164. As shown in FIG. 5C, a radiopaque marker band 186 is embedded in the polymer tubing near the distal end of shaft 164. Also, a distal section 188 of tubing is placed at the distal end of shaft 164 with a slightly reduced inner diameter, as explained further below. As shown in FIGS. 5B and 5C, the metal braid ends adjacent marker band 186 (or overlaps with the marker band and terminates after), and distal section 188 is free of metal braiding in this embodiment. As described further below, the composition of the polymer tubing included in the shaft can vary along the length of shaft 164, for example, to increase flexibility of the shaft toward the distal end of the shaft. In some embodiments, different adjacent sections of polymer tubing can be heat bonded together and further supported with an overarching metal braiding and/or coil reinforcing the majority of the shaft. In some embodiments, the majority of the shaft 164 can have a constant inner diameter, except for distal section 188, to provide for the application of suction through the suction extension positioned at any location within the guide catheter proximal to distal section 188. But in alternative embodiments, a proximal section of shaft 164 can have a larger diameter if desired since the proximal section of the guide catheter may not be used for positioning the connecting section of the suction extension for the application of suction. Appropriate markers on the control wire can be used to ensure that the suction extension is positioned properly for the application of suction.

A lubricious coating, for example, a hydrophilic coating, can be placed on the outer surface of shaft 164 or a portion thereof. Suitable hydrophilic coatings include, for example, polyvinyl alcohol, heparin based coatings, or the like. Hydrohylic coating solutions are commercially available, such as LUBRICENT® (Harland Medical Systems, MN, USA) or SERENE™ (Surmodics, Inc, MN, USA). Further description of the materials and manufacturing process are provided below.

The guide catheter can have an outer diameter (D) from about 5.5 Fr (1.667 mm diameter) to about 10 Fr (3.333 mm diameter), in further embodiments from about 6 Fr (1.833 mm diameter) to about 9 Fr (3 mm diameter), and in some embodiments from about 6.25 Fr (2 mm diameter) to about 8.5Fr (2.833 mm diameter). The guide catheter measurement are generally referenced to the outer diameter, and the inner diameter is less than the outer diameter by twice the wall thickness. In general, the inner diameter of the main portion of shaft 164 (d₁) can range from about 0.8 mm to about 3.175 mm, in further embodiments from about 0.9 mm to about 2.85 mm and in additional embodiments from about 1.00 mm to about 2.7 mm. The reduction in inner diameter of distal section 188 (d₂) relative to the inner diameter of an engagement section of shaft 164 (d₁) can be from about 0.034 mm (0.00134 in) to about 0.25 mm (0.0098 in) and in further embodiments from about 0.05 mm (0.002 in) to about 0.20 mm (0.0079 in). The length of the guide catheter shaft can be from about 30 cm to about 150 cm, in further embodiments from about 35 cm to about 130 cm and in additional embodiments from about 40 cm to about 120 cm and is generally selected to be suitable for the corresponding procedure. In some embodiments, distal section 188 can have a length (L_{d}) from about 1 mm to about 50 mm, in further embodiments from about 1.5 mm to about 25 mm, and in other embodiments from about 2 mm to about 20 mm. A person of ordinary skill in the art will recognize that additional ranges of dimensions within the explicit ranges above are contemplated and are within the present disclosure.

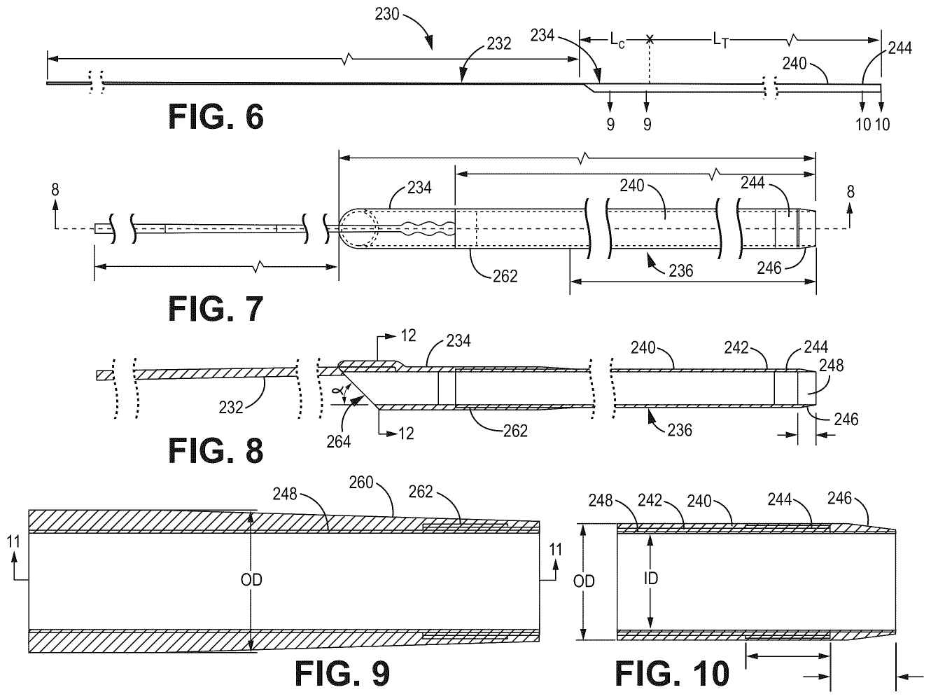

An embodiment of a suction extension is shown in FIGS. 6-12. Referring to FIG. 6, suction extension 230 comprises a control wire 232, connecting section 234 and tubular extension 236. Connecting section 234 connects with control wire 232, which extends in a proximal direction from the connecting section, and tubular extension 236, which extends in a distal direction from the connecting section. In general, control wire 232 can be a solid wire, coil or the like that provides for transmission of pulling and pushing forces to connecting section 234, which correspondingly can move with the tubular extension 236 relative to a guide catheter in the assembled suction catheter system. Control wire 232 can have any reasonable cross sectional shape, which can be different at different locations along the length of the control wire. Also, the control wire can be tapered to a smaller circumference toward the distal end of the control wire. Generally, control wire 232 is made of biocompatible metal, such as stainless steel, titanium or the like, although other materials that have appropriate balance of rigidity and flexibility can be used in principle. In some embodiments, the control wire is a round metal wire with an average diameter along its length from about 0.010 inches (0.254 mm) to about 0.040 inches (1.01 mm) and in further embodiments from about 0.0125 inches (0.32 mm) to about 0.030 inches (0.76 mm). The length of control wire 232 is generally somewhat longer than the guide catheter so that the guide wire extends from the proximal end of the guide catheter, such as 5 cm or more longer than the guide catheter. A person or ordinary skill in the art will recognize that additional ranges within the explicit dimensional ranges above are contemplated and are within the present disclosure.

Connecting section 234 generally is distinguishable by a larger outer diameter than tubular extension 236, and tubular extension 236 extends from the connecting section 234 in a distal direction. In the embodiment of FIGS. 6-12, tubular extension 236 has an approximately constant outer and inner diameter, and a further embodiment is described below with a step down diameter along the tubular extension. Referring to a sectional view in FIG. 10, tubular extension comprises a polymer tube 240, a metal coil reinforcement 242 and a radiopaque marker band 244. Metal coil reinforcement 242 can comprise a flat metal wire, which can extend in some embodiments from roughly radiopaque marker band 244 to a radiopaque marker band in connecting section 234, described further below, although the metal coil reinforcement can extend over the marker bands. Polymer tube 240 can remain the same along the length of tubular extension 236, or the polymer can be changed as different positions along tubular extension 236, for example, getting more flexible in a distal direction. Different sections of polymer can be heat bonded during construction, and metal coil reinforcement 242 as well as optionally a polymer overlayer can further stabilize connected sections of polymer tubing. A tip 246 of tubular extension 236 distal to radiopaque marker band 244 can comprise polymer tubing 240 free of metal reinforcement. A low friction liner 248, such as PTFE or other fluoropolymer, can extend along the length of tubular extension 236 and/or connecting section 234, or portions thereof.

The relationship of connecting section 234 with control wire 232 and tubular extension 236 are shown in FIGS. 6-8. Sectional views of portions of connecting section 234 are shown in FIGS. 9, 11 and 12 and show certain details of the structure. Connecting section 234 can comprise polymer tubing 260 and radiopaque marker band 262. Polymer tubing 260 has a proximal opening 264 that can be angled relative to a longitudinal axis of the polymer tubing to facilitate delivery of devices through the suction extension, although a right angle can be used if desired. The angle α is marked on FIG. 8 and can range from 25 degrees to about 85 degrees, in further embodiments from about 30 degrees to about 80 degrees, and in additional embodiments from about 33 degrees to about 75 degrees. A person of ordinary skill in the art will recognize that additional ranges of angles within the ranges above are contemplated and are in the present disclosure.

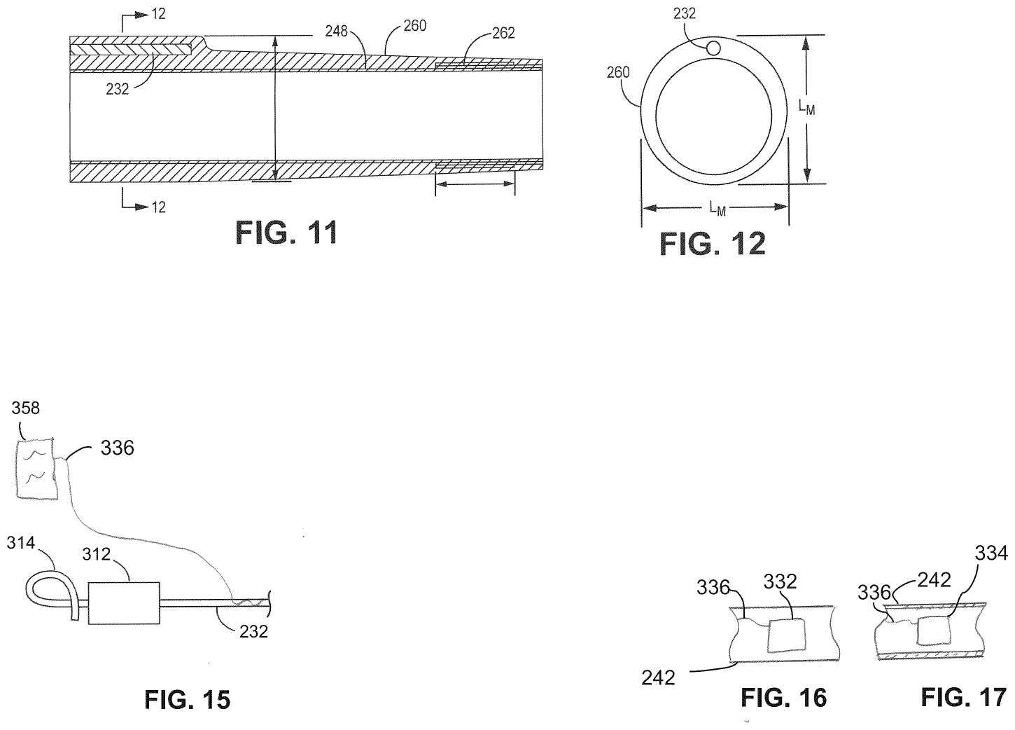

The interface of control wire 232 with connecting section 234 can serve the purpose of both securing the components together as well as helping to form the shape of connecting section 234, which can be selected to provide a desired interface with the interior of the guide catheter lumen. Specifically, the connection of the control wire with the connecting section can facilitate the formation of the oval cross section of the connecting section. In alternative embodiments, control wire 232 can terminate with a flat wire coil that is embedded into a polymer tube to substantially maintain the shape of the connecting section, as described in the ′915 patent and below. In additional or alternative embodiments, an oval shape of the connecting section can be introduced through the molding or other shaping of the polymer which may or may not be combined with a bump due to an embedded control wire. Suitable dimensions of the oval cross section and the processing to form the connecting section are described further below. Low friction liner 248 can extend through the inner lumen of connecting section 234, as shown in FIGS. 9 and 11, or in some embodiments a separate low friction liner can be included in connecting section 234 if desired.

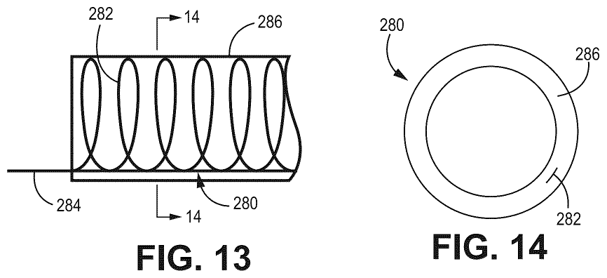

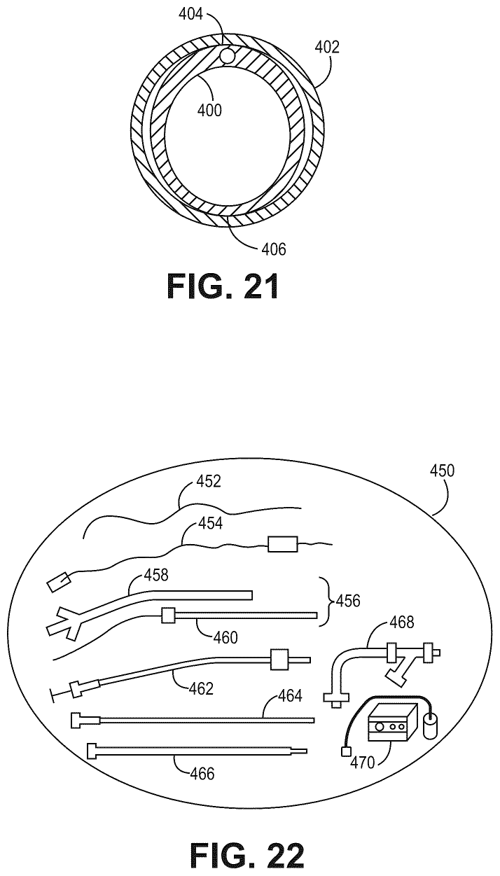

Referring to FIGS. 8, 11 and 12, the distal end of control wire 232 is embedded in polymer associated with polymer tubing 260. Supplementing the polymer wall to secure control wire 232 alters the cross sectional shape that results in a major axis (L_{M}) greater than the minor axis (Lₘ), as can be seen clearly in FIG. 12. As noted above, the non-circular cross section is advantageous for the interface of the suction extension with the guide catheter. The cross section of an alternative embodiment of a connecting section 280 with a non-circular shape is shown in FIGS. 13 and 14. In this embodiment, a flattened metal coil 282 at the end of a control wire 284 is embedded in a polymer tube 286 with a noncircular cross section. The non-circular cross section is formed in this embodiment through forming the polymer with a thicker wall along one edge of the circumference, as can be seen in the sectional view of FIG. 14. A corresponding circular embodiment is shown in FIGS. 21 and 22 of the ′915 patent. The connecting section may or may not have an approximately constant outer diameter over its length, and the outer diameter may taper, e.g. a gradual taper, stepwise taper or combination thereof, over at least a portion of its length to roughly the outer diameter of the adjacent section of the tubular extension.

In some embodiments, a low friction liner, such as PTFE or other fluoropolymer, can extend along the lumen of connecting section 234 and/or tubular extension 236 or selected fractions thereof. Metal reinforcement, such as a flat metal wire coil, can reinforce polymer tube 240 or a fraction thereof. In some embodiments, tubular extension 236 can comprise a first tubular section, a taper section and a second tubular section having a smaller diameter than first tubular section. The taper section tapers between the diameter of first tubular section and the diameter of the second tubular section. This embodiment is discussed further in the ′243 application.

A significant aspect of the suction extension is the narrower diameter suction tip relative to the guide catheter, and a step down diameter of a second tubular section of the alternative embodiment summarized above allows for further reach into narrow neurovascular vessels. The effective suction lumen then extends through the guide catheter into the connecting section of the suction extension and then into the tubular extension, which can have further step downs in diameter. The inner diameter of the connecting section may or may not be the same as the inner diameter of the first tubular section. The narrow diameter of the tubular extension provides for reach into small circuitous blood vessels and the use of the larger diameter proximal suction lumen improves the suction performance significantly without detracting from the ability to reach appropriate locations.

Referring to FIG. 15, control wire 232 has a handle 312 secured near its proximal end. Handle 312 may or may not comprises structure to provide for disengagement of the handle. A specific embodiment of a handle is described in detail below. Control wire 232 can have a twist 314 at its distal end to inhibit the removal of handle 312 from control wire 232. Twist 314 can refer to or be replaced with a bend, a knot, an anchor, or other structure or distortion that prevents or inhibits the removal of handle 312 from control wire 232.

To facilitate monitoring of the pressures and flows through the aspiration system, various components of the aspiration system can be instrumented with sensors. Referring to FIGS. 16 and 17, polymer tube 240 can be provided with one or more pressure sensors. Pressure sensor 332 may be positioned to measure the pressure exterior to polymer tube 240, such as within a vessel. Pressure sensor 334 is positioned to measure the pressure within polymer tube 240. In embodiments, pressure sensors 332,334 may be positioned at or near a distal tip of the tubular shaft. In embodiments, pressure sensors 332,334 may be positioned anywhere along polymer tube 240. In some embodiments, pressure sensors 332,334 may be strategically positioned to have an acceptable impact upon the flexibility of the polymer tube as it is advanced through the vasculature. Wiring for the sensors can be embedded within a polymer wall or otherwise tracked along the length of the tubular shaft to a location near its proximal end. For example, as shown in FIGS. 15-17, wire 336 connects to pressure sensors 332, 334 and controller/display 358. The pressure readings could also be transmitted wirelessly. Wireless pressure measurements are used in the Pressurewire™ X guidewire from Abbott. In some embodiments, wire 336, for example, may terminate at a transceiver that is in wireless communication with controller/display 338. Various suitable pressure sensors can be adapted for use in these devices. Integrated circuit pressure sensors can be used such as the Infineon KP236 pressure sensor. A piezoresistive pressure die P330 W is available from Nova®Sensor with a thickness of 120 microns. These sensors can be appropriately embedded into the wall of the catheter to secure the sensors with appropriate modification of the catheter wall. The use of these pressure sensors in a catheter is described in published U.S. patent application 2018/0010974A to Bueche et al., entitled “Pressure Sensor System,” incorporated herein by reference.

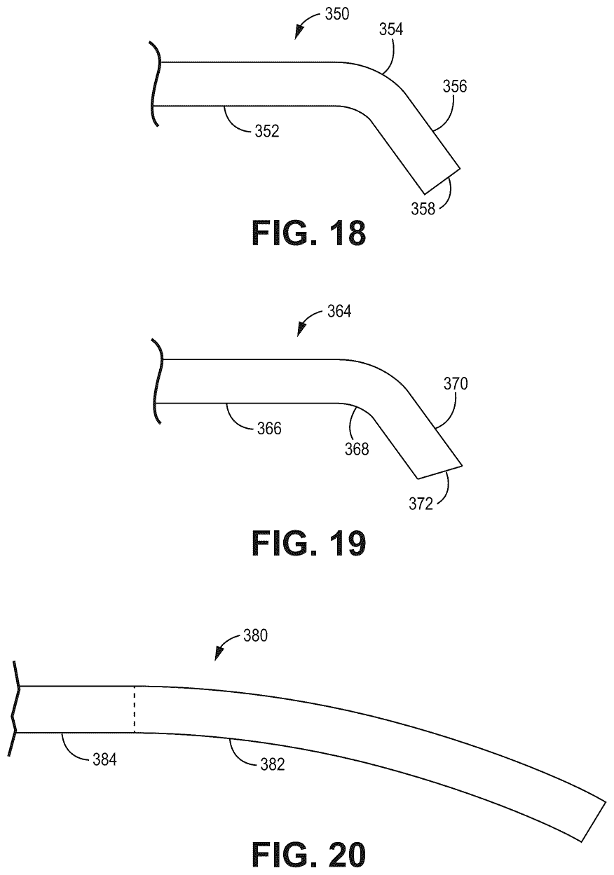

The distal tip of the tubular extension can be bent or curved in its natural unstressed configuration. It has been found generally that a bent tip catheter can facilitate tracking of the catheter over a guidewire without adversely altering the suction abilities. See, for example, U.S. Pat. No. 8,021,351 to Boldenow et al., entitled “Tracking Aspiration Catheter,” incorporated herein by reference. Two general versions of a bent suction tip are shown in FIGS. 18-20. Referring to FIG. 18, suction tip 350 comprises a straight section 352, bend 354 and bent tip section 356 with a flat distal opening 358 approximately perpendicular to the axis of bent tip section 356. Referring to FIG. 19, suction tip 364 comprises a straight section 366, bend 368 and bent tip section 370 with an angled distal opening 372 at a non-perpendicular angle to the axis of bent tip section 370. Bent tip sections 356, 370 are generally cylindrical and can have approximately the same diameters as corresponding straight sections 352, 366. Relative to the embodiment in FIG. 18, the embodiment in FIG. 19 has an angular opening relative to the axis of the catheter. While two shapes of openings are shown in FIGS. 18 and 19, any reasonable shape of the opening generally can be used.

Another embodiment of a bent tip for a suction extension 380 is shown in FIG. 20. In this embodiment, the distal tip 382 is curved with no straight section at the distal end in this embodiment, although alternative embodiments can have short straight segment at the distal end. Distal tip 382 extends from a straight section 384 of suction extension 380. The arc of the curve is approximately circular, but other gentle arcs can be used, in which case the radius of curvature can be an average over the arc. In this embodiment, the curvature of the tip is gradual so that the distal tip may not have a straight section. An angle 7 can be defined based on the point of initial curvature and the natural position of the tip taken at the middle of the distal opening. In some embodiments, angle 7 can be from about 5 degrees to about 21 degrees and in further embodiment from about 7 degrees to about 20 degrees. A person of ordinary skill in the art will recognize that additional ranges of angles within the explicit ranges above are contemplated and are within the present disclosure.

Referring to FIG. 21, a sectional view is shown of a connecting section 400 of a suction extension within an engagement portion 402 of a guide catheter. The non-cylindrical nature of the cross section of connecting section 400 is readily visible. Due to the interface between the elements, the oval shape of connecting section 400 can be distorted relative to its shape separated from the guide catheter, especially if the undistorted length of the major axis of the connecting section 400 is greater than the inner diameter of engagement portion 402. Connecting section 400 can contact the inner surface of the lumen of engagement section 402 at two contact locations 404, 406. The size of contact locations 404, 406 generally depends on the dimensions of the elements, the shape of connecting section 400 and the material properties. It is generally not necessary to precisely define the boundaries of the contact locations.

As noted above, the non-cylindrical connecting section can be characterized with the major axis, minor axis and an average diameter obtained from the circumference. Based on these parameters, it is possible to specify significant aspects of the interface between connecting section 400 and engagement portion 402 with a difference between the major axis and the minor axis, with a difference between the major axis of an unconstrained connecting section 400 and the inner diameter of engagement section 402, and with the difference between the inner diameter of engagement section 402 and the average diameter of connecting section 400. For example, the difference between the major axis and the minor axis can be from about 30 microns to about 160 microns and in further embodiments from about 50 microns to about 140 microns. In some embodiments, the tolerance measured as a difference between the diameter of the inner surface of engagement section 402 and the average diameter of the connecting section can be, for example, no more than about 4 thou (1 thou= 1/1000 of an inch; 4 thou˜102.6 microns), in further embodiments no more than about 3 thou (76.2 microns), in additional embodiments no more than about 1.75 thou (45 microns), in other embodiments from about 1 thou (25.4 microns) to about 1.75 thou (45 microns) and can be approximately zero within the measurement uncertainty. For embodiments in which the major axis of the connecting section separated from the guide catheter is larger than the guide catheter inner diameter, the difference between the major axis of unconstrained (i.e., separated from the guide catheter) connecting section 400 and the inner diameter of engagement section 402 can be from about 0 to about 250 microns, in further embodiments from about 15 microns to about 150 microns and in other embodiments from about 20 microns to about 100 microns. A person of ordinary skill in the art will recognize that additional ranges of dimensions differences within the explicit ranges above are contemplated and are within the present disclosure. Additional embodiments, including embodiments with actuatable structures, are described further in published U.S. patent application 2023/0248377 to Wainwright et al., entitled “Suction Catheter Systems With Designs Allowing Improved Aspiration and Evaluation of Aspiration Conditions,” incorporated herein by reference.

Catheter components can be formed from one or more biocompatible materials, including, for example, metals, such as stainless steel or alloys, e.g., Nitinol*, or polymers such as polyether-amide block co-polymer (PEBAX®), nylon (polyamides), polyolefins, polytetrafluoroethylene, polyesters, polyurethanes, polycarbonates, polysiloxanes (silicones), polycarbonate urethanes (e.g., ChronoFlex AR@), mixtures thereof, combinations thereof, or other suitable biocompatible polymers. Radio-opacity can be achieved with the addition of metal markers, such as platinum-iridium alloy, tantalum, tungsten, gold, platinum-tungsten alloy or mixtures thereof, such as wire or bands, or through radio-pacifiers, such as barium sulfate, bismuth trioxide, bismuth subcarbonate, powdered tungsten, powdered tantalum or the like, added to the polymer resin. Medical grade PEBAX is available commercially loaded with barium sulfate, as well as with ranges of Shore hardness values. Generally, different sections of aspiration catheter can be formed from different materials from other sections, and sections of aspiration catheter can comprise a plurality of materials at different locations and/or at a particular location. In addition, selected sections of the catheter can be formed with materials to introduce desired stiffness/flexibility for the particular section of the catheter. Similarly, fitting components can be formed form a suitable material, such as one or more metals and/or one or more polymers.

In some embodiments, the guide catheter, suction extension or appropriate portions thereof comprises a thermoplastic polymer, such as the polymers listed above, with embedded metal elements, which reinforces the polymer. The wire can be braided, coiled or otherwise placed over a polymer tubing liner with some tension to keep the wire in place over the tubing liner. In some embodiments, a polymer jacket, such as a heat shrink polymer, can then be placed over the top and heated to shrink and fuse the cover over the structure, and/or the polymer tube can be softened with heat to allow incorporation of the metal reinforcements. The wire adds additional mechanical strength while maintaining appropriate amounts of flexibility. The wire can provide some radio-opacity although radiopaque bands generally would provide a darker and distinguishable image relative to the wire. However, the image of the wire can provide further visualization of the catheter during the procedure. To decrease the chance of accidental removal of the radiopaque band from the catheter and to decrease the chance of the radiopaque band catching onto other objects within the vessel, a metal reinforcing wire can be used to cover or enclose the radiopaque band with the metal wire subsequently being embedded within the polymer. In some embodiments, a polymer jacket can be placed over the metal wire, which is correspondingly covering the radiopaque band(s), and the heat bonding embeds the radiopaque marked band also.

The suction system described herein can be used effectively to remove blood clots from the vasculature, including the vasculature of the brain to treat acute stroke conditions. In particular, the narrow tip catheter of the ′792 patent have performed well in human clinical trials to restore blood flow in persons with an acute embolic stroke with good patient outcomes. The device described herein may be expected to provide even better suction while maintaining access capability into vessels challenging to navigate. Nevertheless, for some acute stoke conditions or other embolic events, it can be desirable to use the suction catheter systems described herein with other medical tools for performing the therapy. Furthermore, specific desirable embodiments of proximal fittings are described in this section that provide for improved procedures for use of the suction extension described herein. In particular, the proximal fittings can be adapted with a pressure sensor and/or a flow sensor that can provide valuable information about the status of the suction process. The availability of the pressure and/or flow information can be used to improve aspects of the procedure to increase efficacy and to reduce potential risks to the patient.

Referring to FIG. 22, a treatment system 450 is shown comprising a guidewire 452, embolic protection system 454, suction catheter system 456, shown with guide catheter 458 and suction extension 460 separated, a percutaneous medical device 462, a microcatheter 464, a delivery catheter 466, proximal fittings 468, negative pressure device, e.g., pump or syringe, or the like, 470, and a display unit 472. Suitable components of proximal fittings 468 are described below.

Not all embodiments of medical systems may have all of these components, and some medical system embodiments may have multiple components of each type, such as multiple distinct percutaneous medical devices. Suitable structures covering desirable embodiments for proximal fittings 468 are discussed in the following section. Suitable negative pressure devices include, for example, syringes, pumps, such as peristaltic pumps, piston pumps or other suitable pumps, aspirator/venturi, or the like. Suitable pumps are available from Allied Healthcare Products, Inc., such as a Gomco™ brand pump, or a DRE DM-660™ pump.

Guidewires suitable for use in tortuous bodily vessels are described in published U.S. Pat. No. 10,518,066 to Pokorney et al., entitled “Medical Guidewires for Tortuous Vessels,” incorporated herein by reference. In some embodiments, embolic protection system 454 can comprise a guide structure to provide for delivery of the device, and for these systems a separate guidewire may or may not be used. Suction catheter systems 456 are described in detail herein, and the various embodiments described herein can be adapted for use with the medical systems as well as for use as stand-alone devices. If desired for particularly challenging device delivery, the medical system can include a delivery catheter 466, as described in the ′915 patent.

Embolic protection devices with small filter longitudinal extent and designed for suitable manipulations to facilitate delivery in vessels have been developed that are suitable for use in the medical systems described herein. See, for example, U.S. Pat. No. 7,879,062B2 to Galdonik et al., entitled “Fiber Based Embolic Protection Device,” and U.S. Pat. No. 8,092,483B2 to Galdonik et al., entitled “Steerable Device Having a Corewire Within a Tube and Combination with a Medical Device,” both of which are incorporated herein by reference. Additional fiber-based filter devices particularly designed for delivery into tortuous vessels are described in U.S. Pat. No. 8,814,892B2 to Galdonik et al. (hereinafter the ′892 patent), entitled “Embolectomy Devices and Method of Treatment of Acute Ischemic Stroke Condition,” incorporated herein by reference. The ′892 patent describes the use of the filter device as a clot engagement tool for use with an aspiration catheter. The ′892 patent also envisions the use of supplementary structures to facilitate engagement of the clot. The DAISe™ thrombectomy system system with a fiber based filter is under development by MIVI Nueroscience, Inc. The use of supplementary structures are also contemplated in procedures described herein.

Microcatheters have been designed to allow for access to small blood vessels, such as cerebral blood vessels, and cerebral microcatheters are available commercially, e.g. Prowler Select™ (Cordis Neurovascular Inc.) and Spinnaker Elite™ (Boston Scientific Co.). Of course the term microcatheter can cover a range of devices, and the present discussion can focus on catheters useful for the procedures described herein. In some embodiments, microcatheters can comprise a distal section that is narrower than a proximal section. However, in further embodiments, a microcatheter can have an approximately constant diameter along its length to facilitate delivery of other devices over the microcatheter. A narrow distal diameter allows for the catheter to navigate the tortuous vessels of the brain. The distal section can be highly flexible enough to navigate the vessels, but resilient enough to resist kinking. A microcatheter comprises at least one lumen. The microcatheter can then be used to deliver other treatment devices, aspiration, therapeutic agents, or other means of treating a condition. While microcatheters can have a selected size, in some embodiments, the microcatheters can have a distal outer diameter from about 1.0Fr to about 3.5Fr and in further embodiments from about 1.5Fr to about 3Fr, and a length from about 30 cm to about 200 cm and in further embodiments from about 45 cm to about 150 cm. A person of ordinary skill in the art will recognize that additional size ranges within the explicit ranges above are contemplated and are within the present disclosure.

With respect to percutaneous medical devices 462, suitable devices include, for example, clot engagement devices, angioplasty balloons, stent delivery devices, atherectomy devices, such as stent retrievers, and the like. Desirable thrombus engagement devices are described in U.S. Pat. No. 10,463,386 to Ogle et al., entitled “Thrombectomy Devices and Treatment of Acute Ischemic Stroke With Thrombus Engagement,” incorporated herein by reference. Stents may be, for example, balloon extendable, self-extendable or extendable using any other reasonable mechanism. Also, balloon extendable stents can be crimped to the balloon for delivery to engage a clot in a blood vessel. Some balloon-stent structures are described further, for example, in U.S. Pat. No. 6,106,530, entitled “Stent Delivery Device;” 6,364,894, entitled “Method of Making an Angioplasty Balloon Catheter;” and 6,156,005, entitled “Ballon [sic] Catheter For Stent Implantation,” each of which are incorporated herein by reference. Self-expanding stents are described further in U.S. Pat. No. 8,764,813 to Jantzen et al., entitled “Gradually Self-Expanding Stent” and U.S. Pat. No. 8,419,786 to Cottone, Jr. et al., entitled “Self-Expanding Stent,” both of which are incorporated herein by reference. Stent retrievers are described, for example, in U.S. Pat. No. 8,795,305 to Martin et al., entitled “Retrieval systems and methods of use thereof,” incorporated herein by reference.

Proximal fittings 111 provide both isolation of the catheter interior as well as connection of the catheter interior to devices to control flow of fluids. Referring back to FIG. 2, proximal fittings 111 provide a manifold that allows for functionality that facilitate the procedure. For example, hemostatic valve 123 can control access to the clean plenum within the catheter interior as well as limit any blood loss due to leakage. Inventive procedures can be implemented using adaptive aspiration supply 121.

To facilitate use of short aspiration catheters that share an aspiration lumen with the guide catheter, adaptations of the proximal fittings can provide for removal of a tubular extension of the suction extension from the guide catheter without passage through a hemostatic valve. These configurations are described further in the ′723 patent. In some embodiments, the proximal fittings can further comprise an additional branched fitting with a proximal end that can dock the proximal end of the suction extension to provide for convenient removal from the isolated locations behind a hemostatic valve to provide for convenient clearing of thrombus blockage of the suction extension and reinsertion. The thrombus blockage can be cleared through a flush delivered from a branch of docking Y-connector with the suction extension docked for quick replacement of the suction extension for the additional removal of further blockage form the blood vessel in the patient. The fittings with a docking structure are described in the ′865 patent. These optional embodiments are not discussed further here.

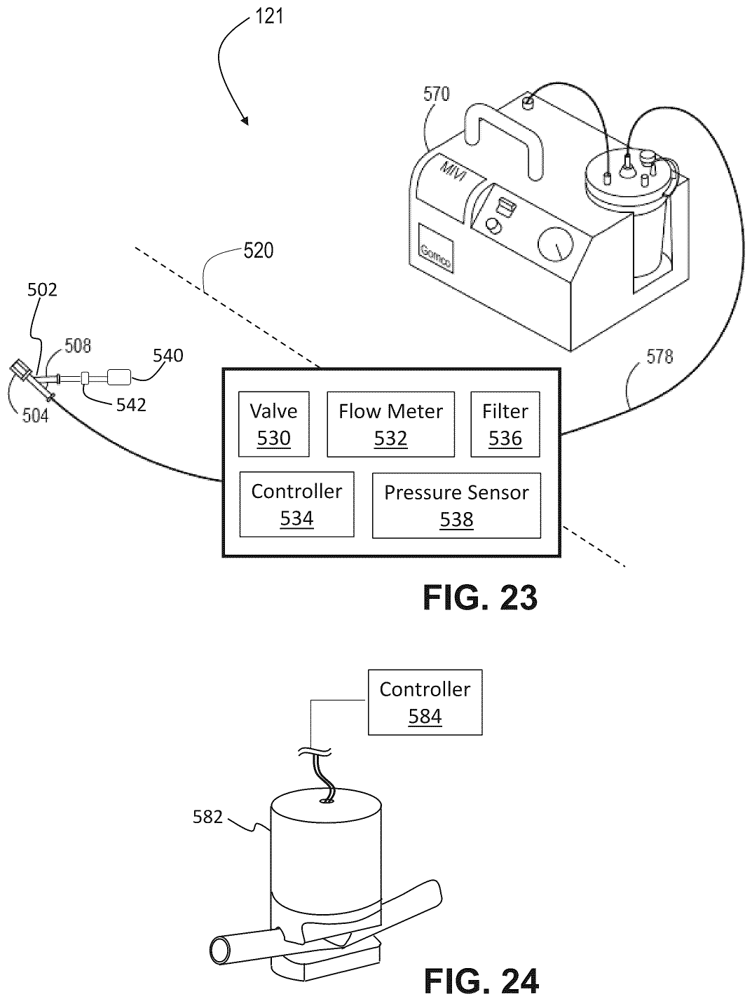

The general set up of an adaptive aspiration supply 121 is depicted in FIG. 23. A manifold 502 is shown for connecting to proximal fittings 111, although other configurations are possible including an integral connection with the proximal fittings. Manifold 502 has a connector 504, such as a luer connector component, and two branches 506, 508. A manifold can have more than two branches or a single branch in some embodiments. As shown in FIG. 23, branch 506 is connected to pump 508 using tubing 510, which can be high pressure tubing. Under regulatory guidelines, placing components a sufficient distance from the patient and under appropriate sterile procedures. Such a demarcation by distance is noted in FIG. 23 as border 520, which is shown as a dashed line. Components on the patient side of border 520 generally would be single use or require other special care since they can be presumed to be contaminated by fluids from the particular patient. Components on pump side of border 520 may be reused, although maintained sufficiently sterile to avoid infection for the patient.

The adaptive aspiration supply 121 generally further comprises at least one of an automatically controlled valve 530, a flow meter 532, a controller 534, a filter 536 and optionally a pressure sensor 538. As shown in FIG. 23, these components are shown in a box overlapping boundary 520 since they can be potentially placed on either side of the boundary, which may correlate with single use status. The order of the components can influence the sensor readings, and particular placement order is discussed in the following discussion. Two embodiments are exemplified. Prior to discussing the assembled embodiments of the adaptive aspiration supply 121, embodiments of these individual components are discussed.

During pulsatile flow, a valve to the pump may be closed. It has been proposed to expose the catheter to atmospheric pressure or other purge fluid pressure during this time to perhaps allow a clot to relax relative to the low pressure status when the valve is closed. This can be accomplished through branch 508 of FIG. 23. An alternative pressure system can be at a range of pressures, and this can be described in terms of a fluid reservoir to deliver the selected pressure. In some embodiments, the alternative pressure system can maintain a negative pressure, but at a significantly lower magnitude than supplied by the pump. Referring to FIG. 23, a fluid reservoir 540 is connected to branch 508. Valve 542 controls fluid communication between fluid reservoir 540 and branch 508. In some embodiments, fluid reservoir 540 can be at atmospheric pressure, or at a higher pressure. In other embodiments, fluid reservoir can be at a negative gauge pressure, but substantially less in magnitude relative to the pump negative pressure.

While controller 534 can be manually controlled, full implementation of the automated features of the procedures described herein rely on a controller operated under programmed control. In general, controller 534 may receive input regarding measurements of other sensors and/or instructions from various user inputs and/or other control processors. Similarly, output from controller 534 can be directed to various displays and/or other processors to facilitate control of the overall process.

While valve 582 can be a manually controlled valve, such as a pinch valve actuated with a lever, electronically controlled valves can provide easier and faster control of the valve, and electronically controlled valves can implement the automated aspects of the processes described herein. In particular, a solenoid valve can be a desirable design. While generally other electronic valves can be used as desired, commercially available solenoid valves can be attached to the exterior of the tubing for convenient use without contaminating either the flow or the valve. A commercial solenoid valve for mounting on tubing is available from Cole-Palmer® under the Masterflex series of two-way solenoid-pinch valves. A proportional solenoid pinch valve is available from IMI Norgren® under the Acro 900 series. As shown in FIG. 24, valve 582 is connected to controller 584.

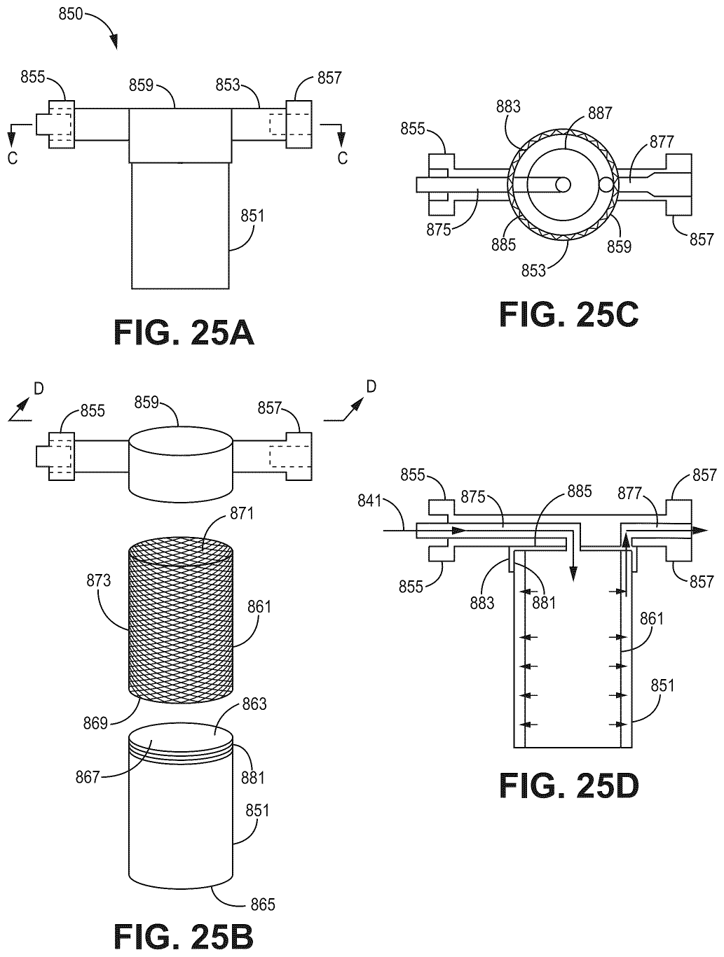

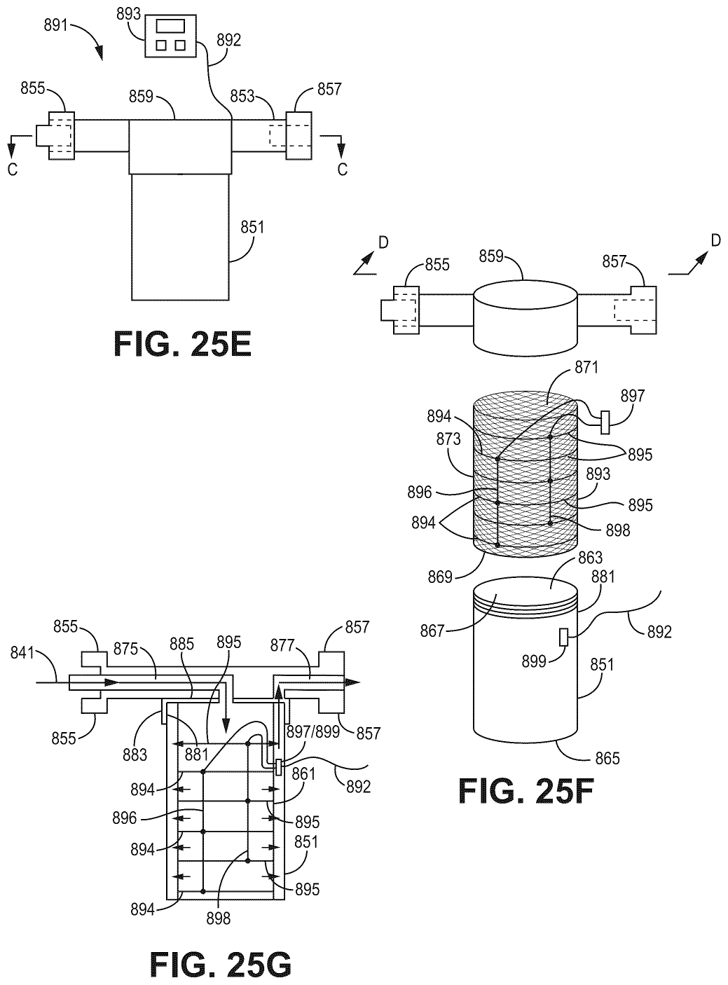

Referring to FIGS. 25A-25D, filter 850 has filter body 851 and end cap 853. End cap 853 includes connections 855, 857 configured to attach with high-pressure tubing and proximal fittings. In embodiments, connections 855, 857 are luer fittings. Filter body 851 interfaces with a central portion 859 of end cap 853. In embodiments, filter body 851 and central portion 859 of end cap 853 have corresponding threads 881, 883, respectively, such that filter body 851 may screw into central portion 859 and form a seal. If desired, central portion 859 can comprise a washer or gasket 885 to engage with screwed on filter body 851. Filter body 851 further has an open top end 863 opposite closed bottom end 865, and an interior chamber portion 867 there between. End cap 853 has a first channel 875 extending from a first connection 855 to about the center of central portion 859, such that the channel is in fluid communication with the interior chamber 867 of filter body 851. First channel 875 then bends, e.g. about 90 degrees, towards filter body 851 to direct the flow. End cap 853 has a second channel 877 extending from a second connection 857 to about the perimeter of central portion 859, where second channel 877 bends, e.g. about 90 degrees, towards the edge of filter body 851 outside of area constrained by washer/gasket 883, such that flow outside of screen filter element 861 can flow to second channel 877.

Filter 850 may have a filter element 861 or similar filter structure. Screen filter element 861 is configured to fit within interior chamber portion 867 of filter body 851 and is fully contained therein when end cap 853 is secured to filter body 851. Filter element 861 optionally has a closed end 869 at bottom end opposite open top end 871 and mesh screen 873 there between. In some embodiments, closed end 869 engages the bottom of filter body 851 to restrict clots from exiting screen filter element 861. Closed end 869 can alternatively have a screen to allow flow through the end. Fluid entering, for example, through the open end 871 of filter element 861 passes through the screens 873 in order to exit filter 850. Filter element 861 should be sized to leave an appropriate gap between the filter element 861 and the wall of chamber portion 851 as well as to leave a flow path to the exit of interior chamber portion 851. For example, as shown in FIG. 25D, filter element 861 may have an outer diameter that is less than an inner diameter of chamber portion 851. Flow 841 enters filter element 861 through first channel 875. In embodiments, filter element 861 has a height that roughly matches a height of filter body 851, such that filter element 861 is held in position when filter body 851 is secured to end cap 853. In some embodiments, top end 853 can comprise a washer or the like to engage the top of filter element 861 when filter body 851 is engaged with end cap 853.

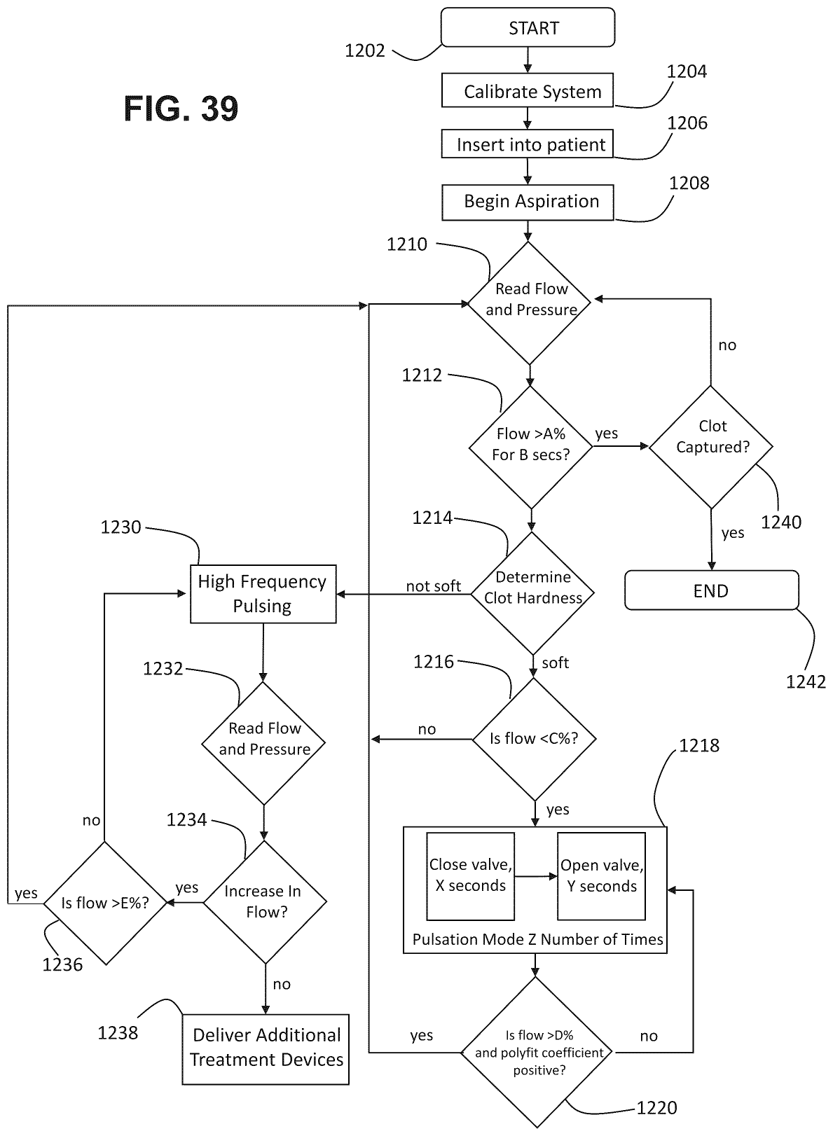

In embodiments, central portion 859 of end cap 853 may have a lip, protrusion and/or gasket that engages the top of filter element 861. A gap should be maintained between the wall of chamber portion 851 and filter element 861 such that flow 841, upon passing through screen 837 may continue between filter element 861 and wall of chamber portion 851, ultimately exiting in line filter 850 through connection 857. It should be recognized that flow 841 can be reversible and filter 850 may work with flow entering connection 857 and exiting through connection 855, but collection of clots is not necessarily equivalent for the two flow directions. A person of ordinary skill in the art can adjust these designs to have other functionally equivalent configurations based on this teaching. For example, the inclusion of O-rings, washers, gaskets, or the like may be used for seals to direct flow 841 and are not beyond the scope of this disclosure. In addition, while FIG. 39C depicts first channel 875 and second channel 877 connecting to inlets and outlets in a linear configuration, there is no functional need for this configuration, and the respective inlets and outlets can be placed at a selected angle relative to each other around the circumference as long as the inlet and outlet do not interfere with each other. The depicted linear configuration can be convenient for a range of setups.

Mesh screens 837 may be sized appropriately to capture clots while letting fluid flow essentially unimpeded. Since the purpose of the mesh screens is to remove clots that can impede flow through the tubing and not to purify blood for the patient, the pore size through the screen need not be particularly small. Pore sizes less than 1 millimeter and in further embodiments less than 0.5 millimeter may be adequate, and generally the pore sizes should not be too small, such as greater than at least about 0.1 mm. Similar effective filter sizes can be considered for the other embodiments. For meshes with relatively large pores, fibers can be included in the filter to help trap the clots, and gravity can further assist with the trapping clots, especially with a configuration, such as shown in FIG. 39. The packing of fibers can be selected to facilitate clot capture without significantly constricting flow or excessively obscuring visualization inside the filter. In embodiments, filter body 851 may be transparent, allowing for a visual assessment of debris trapped within filter 850. The ability to identify whether or not a clot has been captured within the filter, if it is transparent, can improve safety and help to guide the practitioner in performing the procedure.

An instrumented embodiment of the filter is shown in FIG. 25E-G. Referring to FIG. 25E, filter 891 comprises an electrical connection 892 connecting filter 891 with controller 893. Controllers for making electrical measurements on biological fluids are available commercially, such as electrical chips from Analog Devices, Inc., such as AduCM355 chip. Generally, the controller provides a low voltage ac current, but a direct current could be applied. Electrical connection 892 can connect with electrodes within the filter housing in various ways. Generally, electrodes for the measurement can be located on the filter material to provide a relatively direct measurements influenced by clot captured in the filter material. The wires connected to the electrodes pass through the filter container, which can be through cap 859 or filter body 851. If the filter material is not exchanged, the electrodes can be hard wired, or alternatively an optional connecting clip can be used to disconnect the electrodes to allow for replacement of the filter material within the filter cartridge.

FIG. 25F is analogous to FIG. 25B with a filter element 893 having integral electrodes. In the depicted embodiment, filter element has electrodes encircling filter element 893 with three electrodes 894 of a first polarity/phase and three electrodes 895 of a second polarity/phase. Wire 896 connects electrodes 894 with connector element 897, and wire 898 connects electrodes 895 with connector element 897. Wires 896, 898 are appropriately insulated to avoid short circuit. Mated clip element 899 is connected to electrical connection 892 through a sealed hole through filter body 851. Connector elements 897, 899 are optional, and wires 896, 898 can be directly connected to electrical connection 892. Electrodes 894, 895 can be made from electrically conductive features placed along mesh 873, and mesh 873 can be formed from an insulating material, such as polymers (polyamines, polycarbonates, etc.) or ceramics (silicates, etc.) to avoid short circuits. An alternative view is shown in FIG. 25G. The numbers of electrodes and positioning can be altered as desired to achieve appropriate measurement values. With blood flowing through the filter, a certain current/impedance can be measured, which can alter if a clot is trapped by the mesh. Such a change of current or impedance measurement can indicate the presence of a clot, and the degree of change can provide information related to the amount or composition of the clot.

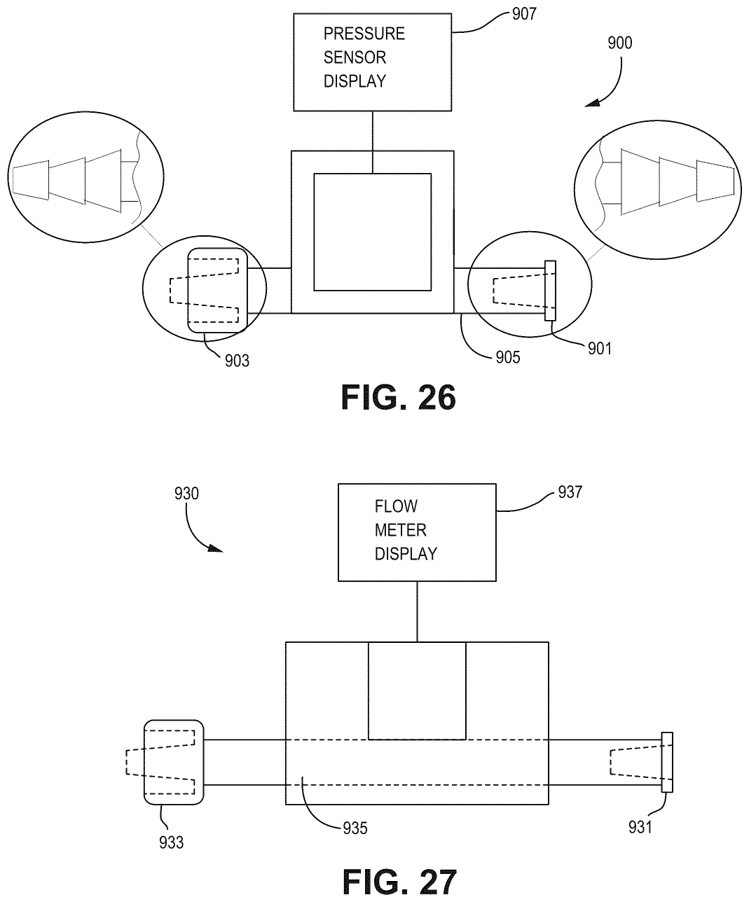

An example of a fitting adapted with a pressure sensor is shown in FIG. 26. Referring to FIG. 26, pressure sensor 900 has a female luer fitting 901, a male luer fitting 903, and a channel 905 there between. As shown in the balloon inserts adjacent fittings 901, 903, the fittings can be replace with tubing connectors 905, 906 to allow pressure sensor 900 to be connected to high pressure tubing of adaptive aspiration supply 121. For other components similar connector replacements can be made, as desired. Pressure sensor 900 may have a display 907 indicating the measured pressure of fluid passing through pressure sensor 900. In embodiments, display 907 may be integral with pressure sensor 900. In embodiments, display 907 may be a separate display unit, for example, connected via electrical connection or a wireless connection to pressure sensor 900. As described in more detail below, in embodiments, display 907 may be integrated into a multi-functional display that can contemporaneously display output from multiple sources during a procedure. Female luer fitting 901 and male luer fitting 903 are in fluid communication with fluid within channel 905. Various designs of a pressure sensor may be suitable. In some embodiments, electrical wires can extend from pressure sensor 900 and terminate at an electrical connector, which can be a multi-pin clip or other suitable connector configuration. An electrical connector can be suitable for connection to the controller and/or a suitable monitor or display. Commercial pressure sensor components for use as pressure sensor 900 are commercially available, for example, from PendoTECH, Princeton, NJ, USA or MPS microfluidic pressure sensors are available from ELVEFLOW (Darwin Microfluidics), which also provides software for their operation. These components can be purchased sterile, or they can be sterilized before use using conventional methods, such as using gamma irradiation.

Referring to FIG. 27, flow meter 930 has a female luer fitting 931, a male luer fitting 933, and a channel 935 there between. Fittings 931, 933 can be replaced with Flow meter 930 has a display 937 indicating the measured flow rate of fluid passing through flow meter 930. In embodiments, display 937 may be integral with flow meter 930. In embodiments, display 937 may be a separate display unit, for example, connected via electrical connection or wireless connection (such as blue tooth) to flow meter 930. As described in more detail below, in embodiments, display 935 may be integrated into a multi-functional display that can contemporaneously display output from multiple sources during a procedure. Readings from flow meter 930 can be simultaneously displayed on multiple display devices. Female luer fitting 931 and male luer fitting 933 are in fluid communication with fluid flowing through channel 935.

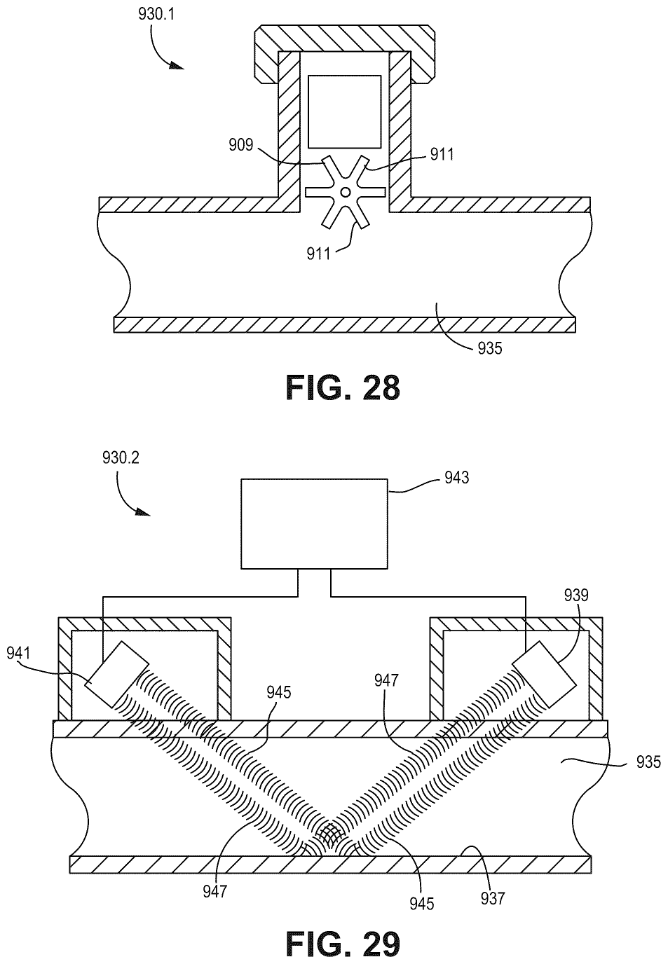

As shown in FIG. 28, in embodiments, flow meter 926 has a paddle wheel 909 positioned such that one or more paddles 911 extend partially into channel 905. Fluid flowing through channel 905 pushes the one or more paddles 911 causing paddle wheel 909 to rotate. A flow rate is associated with the rotational velocity of paddle wheel 909 as it turns.

In an alternative embodiment, as illustrated in FIG. 29, ultrasonic flow meter 928 has a first transceiver 939 and a second transceiver 941. First and second transceivers 939, 941 are in electrical communication with computational unit 943. First transceiver 939 emits a first ultrasonic signal 945 which reflects off an interior surface 937 of channel 935 with modulation from the fluid flow and is received by second transceiver 941. Second transceiver 941 emits a second ultrasonic signal 947 which reflects off an interior surface 937 of channel 935 and is received by first transceiver 939. In embodiments, first and second transceivers 939, 941 are ultrasonic transducers and/or ultrasonic sensors. Computational unit 943 receives output from first and second transceivers 939, 941. In embodiments, computational unit 943 can use output from first and second transceivers 939, 941 to calculate characteristics of fluid flowing through channel 935. For example, computational unit 943 can determine a flow rate of a fluid flowing through 935. Ultasonic flow meters are commercially available for adaptation to these purposes. For example, Dynasonics ultrasonic flow meters (such as Dynasonics DXN flow meter (Badger Meters, Inc., WI, USA) down to 0.5 inch diameter pipe) can be clipped onto a tube to measure flow rate based on Doppler ultrasound effect. Coriolis flow meter can connect directly to the tubing to allow flow through the device, and commercial coriolis flow meters are available, such as BFS Microfluidic Coriolis Flow Sensor (available from DARWIN Microfluidics) and controllable with ELVEFLOW Software.

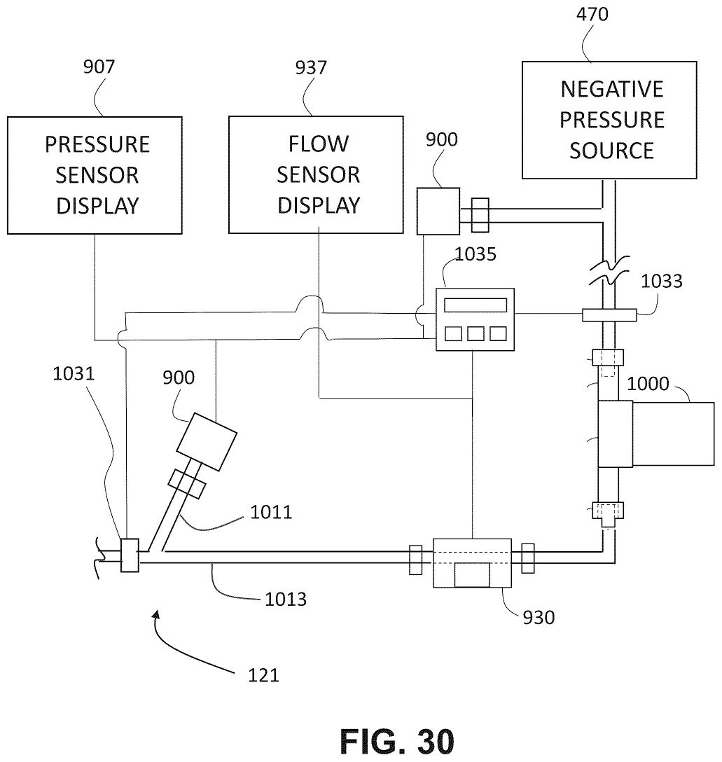

An embodiment of an adaptive aspiration supply 121 is shown in FIG. 30. As shown in the embodiment of FIG. 30, adaptive aspiration supply 121 is connected to fittings 111 of FIG. 2 at connector 510 (504), see also FIG. 23. It can be desirable to transition to high pressure tubing for connection of the components of adaptive aspiration supply 121, although they can maintain connection using standard catheter fitting connectors, such as Luer connectors, if desired. Referring to FIG. 30, adaptive aspiration supply 121 may comprise a first valve 1031, pressure sensor 900, flow meter 930, filter 1000, and a negative pressure source 470. Pressure sensor 900 is connected to pressure sensor display 907 and flow meter 930 is connected to flow sensor display 937. In embodiments, pressure sensor 900 and flow meter 930 are connected to controller 1035. In embodiments, adaptive aspiration supply 121 comprises a Y-branch manifold having a first branch 1011 connected to pressure sensor and a second branch connected to flow meter 930, filter 1000, and negative pressure source 470. In embodiments, first branch 1011 of Y-branch manifold is distal to second branch 1013 of Y-branch manifold. In embodiments, filter 1000 is proximal to flow meter 930. In this particular embodiment, first valve 1031 is positioned distal to first branch 1011. First valve 1031 can be an automatic controlled valve, a manual valve or replaced with a connector, such as a Luer connector if valve 1033 provides sufficient control of the flow to the pump. A second valve 1033 may be located proximal to filter 1000. First valve 1031 and second valve 1033 may be electrically connected to controller 1035.

In some embodiments, the controller 1035 may be a general-purpose integrated microcontroller system. The system may include a central processing unit (CPU) responsible for executing program instructions, volatile and non-volatile memory components for data storage. The microcontroller may further include input/output (IO) ports for interfacing with additional modules, sensors, external devices, and other peripherals. The system may also include a transceiver for wireless communications such as a Bluetooth module. IO ports may be configured to attach directly to displays, or, in some configurations, may attach to other computing systems that may be commonly found as part of surgical suites. IO ports may be further configured to employ use wireless communications as opposed to direct electrical attachment. For example, the controller 1035 may be configured to transmit flow and/or pressure measurements to a wireless display unit. For example, the Arduino® UNO is an integrated microcontroller system of the type described herein.

The embodiment shown in FIG. 30 has one particular configuration of components of the adaptive aspiration supply. Other order of components (pressure sensor, flow rate sensor, filter and automatic valve) can be used, and additional components can be used. The function of the automatic valve can be replaced by the automatic turning on and off of the pump, but generally a valve can provide desirable responsiveness and the availability of greater control on the direction of the aspiration. The closing of a valve can influence the resulting pressure readings, so the placement of a pressure sensor relative to the valve can be significant. Thus, the pressure sensor can be placed on either side or with two pressure sensors on respective sides of an automatic valve. Also, the relative placement of the filter can influence transient flow and pressure readings. Two specific working embodiments are described further below.

The suction catheter system is generally appropriately sterilized, such as with e-beam or gas sterilization. The suction catheter system components can be packaged together or separately in a sealed package, such as plastic packages known in the art. The package will be appropriately labeled, generally according to FDA or other regulatory agency regulations. The suction catheter system can be packaged with other components, such as a guidewire, filter device, and/or other medical device(s). The packaged system generally is sold with detailed instructions for use according to regulatory requirements.

General Aspiration Thrombectomy Procedures

The procedures herein provide significant advantages with embodiments based on adapting the procedure based on flow and/or pressure measurements. In some embodiments, dynamic control of the flow can provide improved efficacy of the procedure relative to embodiments with constant aspiration or with completely manual control. Some improved embodiments based on availability of pulsed aspiration. The procedures are based on measurement of pressure and/or flow to evaluate the status of the clot clearance and control valves and/or direct direction of the procedure. Prior to discussing the improved embodiments of the procedure, it is helpful to outline the basic procedure to provide the context of the discussion.

As indicated above, the medical systems comprising an aspiration catheter system described herein can be used with the aspiration catheter system as stand-alone treatment device, perhaps with a guidewire and/or other delivery support devices, or used with supplemental medical treatment devices for treatment of ischemic vessel blockage. In some embodiments of the improved procedures, the adaptive aspiration supply can provide feedback regarding the desire to introduce other treatment modalities to assist with clot removal. In particular, in some embodiments, the aspiration catheter system is used with an embolic protection device or a hydraulic fluid infusion device, and in additional embodiments, some form of clot engagement device, stent, balloon, atherectomy device or the like may also be used. In any case, a guidewire is generally used to provide access to the treatment site. The guide catheter portion of the suction catheter system may or may not be positioned prior to the introduction of an aspiration catheter.

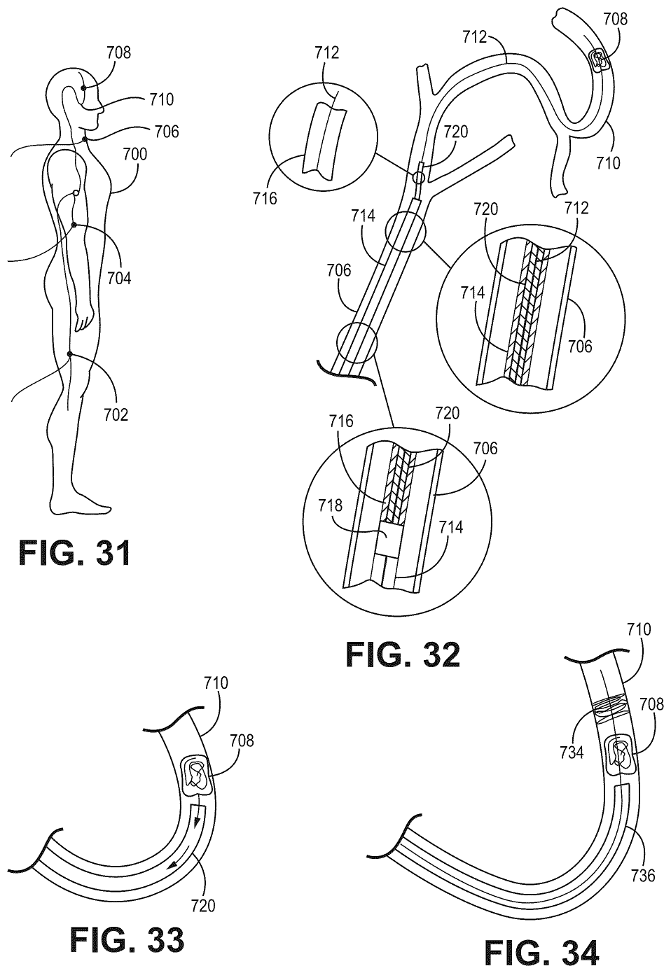

For the treatment of an acute ischemic stroke condition, referring to FIG. 31, a patient 700 is shown with three alternative access points into the vasculature, femoral artery 702, artery in the arm 704 or carotid artery in the neck 706. Regardless of the access point, the catheter and associated devices are guided to the left or right carotid artery to reach a clot 508 in a cerebral artery 710 of the brain. Referring to the schematic view in FIG. 32, clot 708 is shown in cerebral artery 710 with a guidewire 712 positioned with its distal tip past the clot. Guide catheter 714 is positioned over the guidewire within the carotid artery 706. Suction extension 716 with connecting section 718 within guide catheter 714 and tubular extension 720 extending from guide catheter 714 over guidewire 712. Referring to FIG. 33, tubular extension 720 can be advanced over the guidewire to a position near clot 708. Suction can be applied as shown with the flow arrows in the figure. Guidewire 712 may or may not be removed before suction is applied. Aspiration catheters have successfully removed clots responsible for ischemic stroke without further medical devices in the intervention. However, for more difficult clots, additional medical treatment devices can be used as described in detail below.

Using the embodiments of proximal fittings, such as shown above, adapted with pressure sensing capability, the initiation of suction as described in the context of FIG. 33 can be checked with respect to its efficacy. If appropriate flow is established since negative pressure is applied to the catheter system, the pressure in the proximal fittings can be in a suitable range. The precise ranges of expected pressures generally are dependent on the specific design of the suction extension, and the acceptable pressure range can be adjusted accordingly. In any case, the pressure can be confirmed in real time during the procedure for comparison with specifications adapted for the specific suction catheter components. If the pressure at the time immediately following the initiation of suction is closer to the negative pressure of the pump than expected based on the set acceptable range, the physician can withdraw the suction extension at least part way from the delivered configuration with or without stopping suction. A partial withdrawal can be used to try to unkink the suction extension without complete removal. As described further below, if proximal fittings are used that allow removal of the tubular extension for the patient without passing through a hemostatic valve, the tubular extension can be visually checked without exposing the tubular extension to the ambient atmosphere. After verifying that the tubular extension is ready for use or after replacing the suction extension, the suction extension can be redelivered.

When initiating the process, the system is generally primed with sterile saline to remove air from the aspiration system through to the pump. Pressure and flow measurements then relate to liquid parameters, such as the saline and/or blood as blood gets pulled into the system. When using the suction system to clear actual clots associated with acute ischemic stroke events, it is frequently found that the tubular extension becomes clogged itself prior to fully clearing the vessel. Therefore, it can be desirable to clear the clot form the tubular extension and reintroduce the suction extension back into the cerebral vessel to remove additional thrombus. The clearing and reintroduction can be repeated as necessary. The fittings described herein can facilitate this process, and the use of these fittings to effectuate this process are described further below. The desire to clear clots from the suction extension and reintroducing the suction extension may also be performed with the use of additional treatment structures as described in the following.

The use of a flow meter provide a significant additional parameter to guide the procedure. While pressure changes may provide some overlapping information, the additional flow measurements can provide additional guidance. If the flow drops, this can signal that the clot is lodged somewhere or that the suction extension is kinked. Depending on the stage of the procedure, the suction extension/aspiration catheter can be removed from the guide catheter and cleared of any clots. This then allows for the guide catheter to be checked if clear from any blockages. A sudden increase in flow can indicate that the clot has been removed. If the clot is in the filter, this can indicate advance of the procedure, but if the clot is not identified in the filter, the practitioner can carefully check likely alternative locations of the clot and proceed with caution in the procedure to avoid inadvertent redirecting the clot into the patient.

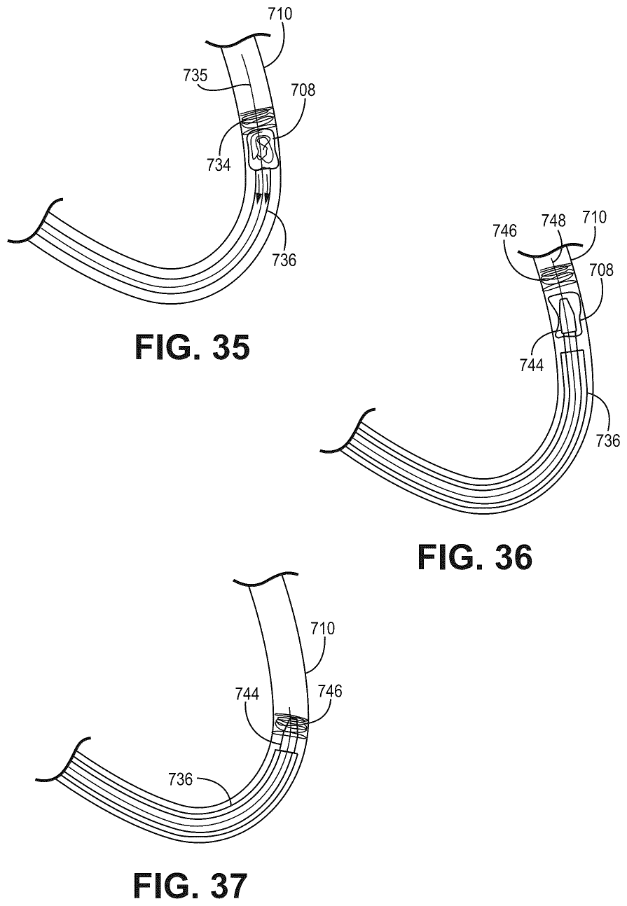

Referring to FIGS. 34 and 35, the use of a fiber-based filter device is shown in use along with the suction catheter system. As shown in FIG. 34, clot 708 is shown in cerebral artery 710 with a deployed fiber-based filter 734 supported on a guidewire 736 positioned with the filter deployed past the clot. Fiber-based filter 734 can have fiber elements extending essentially to the wall of the vessel, cerebral artery 710. Tubular extension 736 can be positioned with its distal tip just proximal to the clot, and the remaining portions of the suction catheter system are not shown in this view. Referring to FIG. 35, fiber-based filter 734 can be pulled toward tubular extension 736 with suction being applied to facilitate removal of clot 730. Clot 708 can be broken up and removed by suction, and/or all or a portion of clot 708 can be pulled into tubular extension 736 optionally along with all or part of the fiber-based filter, and/or all or a portion of clot 708 can be held to the opening of tubular extension 736 with the fiber-based filter holding the clot. In any case, once the clot is appropriately stabilized, the devices and any clot still within the vessel or catheter can be removed from the patient. The removal of the devices is described further below.

The further use of an additional medical device to facilitate clot removal is shown in FIGS. 36 and 37. As shown in FIG. 36, clot 708 is shown in cerebral artery 710 with a medical treatment device 744 positioned at the clot and deployed fiber-based filter 746 supported on a guidewire 748 positioned with the filter deployed past the clot. Suitable medical treatment devices for clot engagement are described above. The selected medical treatment device is deployed generally with protection from the deployed fiber-based filter and optionally with suction. Once the clot is engaged with the medical treatment device, the recovery of the remaining portions of the clot and the medical treatment devices can be removed as shown in FIG. 37, similarly to the process shown in FIG. 36. In particular, the medical treatment device can be removed, although portions such as a stent may be left behind, and the removal can precede or can be done in conjunction with removal of a filter in the blood vessel and/or remaining fragments of clot. All or a portion of clot 708, if not already broken up and removed with suction can be pulled into tubular extension 736 optionally along with all or part of the fiber-based filter, and/or all or a portion of clot 708 can be held to the distal opening of tubular extension 736 with the fiber-based filter holding the clot. Again, once the clot is appropriately stabilized, the devices and any clot still within the vessel or catheter can be removed from the patient. The use of a plurality of additional medical treatment devices can be performed through extension of the procedure outlined above to repeat steps involving the additional medical device.

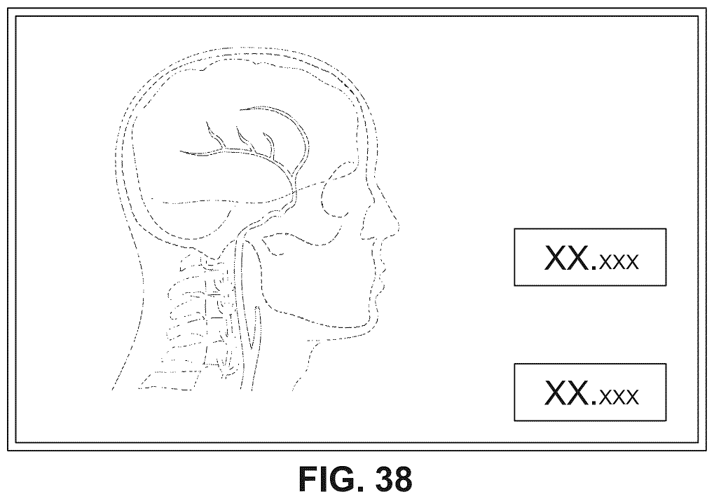

FIG. 38 depicts a video monitor 680 displaying a real time x-ray image 682 of the patient at the site of the thrombus removal along with the pressure value 684 and the flow rate 686. Through having all of these images simultaneously visible, the health care provider can assess all of the information to make a decision regarding next steps of the procedure.

Dynamic Aspiration Adjustment

The modified procedures are intended to shorten, on average, the aspiration time for clots that are readily removed, to facilitate ultimate removal with aspiration of more difficult to remove clots and also to help determine more quickly if additional treatment modalities should be introduced into the procedure. As noted in the ′828 application, the availability of flow and pressure measurements allows for evaluation of the clot status. This information can be effectively used to control delivery of aspiration. With a well designed aspiration catheter, such as the Q-catheters described herein, easier to remove clots can be quickly removed with constant aspiration. For clots that are somewhat more difficult to remove, pulsed aspiration can be applied if it is determined that the clot is not being quickly removed, which can be effective to shorten the average clot clearance time. By still tracking the clot clearance, the aspiration can be quickly terminated to avoid excessive blood loss and reduce risk of vessel damage resulting from transmission of the full aspiration pressure of the pump once the clot is cleared.

A flow chart of the dynamic aspiration adjustment procedure is shown in FIG. 39. At the start 1202 of the procedure, a user signals to the system to begin 1202. Next, calibration 1204 is performed to determine flow rate and pressure ranges corresponding to unrestricted flow and closed flow. Once calibration 1204 is completed, the catheter is placed into the patient 1206 with the tip of the aspiration catheter appropriate imaging, such as real time x-ray imaging with contrast. With the catheter in place in the patient, the user signals to the system to begin aspiration delivery 1208 to the patient. Next, flow rate and/or pressure are measured 1210 from the patient through the catheter. The aspiration procedure continues then until the clot is cleared or the procedure is otherwise terminated. Generally, the user would be able to terminate the procedure manually for potential unforeseeable safety reasons.

Intermediate values of flow rate and pressure can be interpreted with respect to various degrees of corking of the clot. The character of corking can extend over various ranges with varying degrees of flow past the corked clot, which result in corresponding intermediate values of flow and pressure. The flow can be particularly sensitive to fluctuations during corking with oscillations in the flow. The continued flow while corked would seem to correspond with flow getting past the corked clot, which we can call by-pass flow. The pressure tends to remain nearer the no flow limit rather than a more intermediate pressure between the no flow and the free flow limits, so bench simulations suggest less sensitivity of the pressure measurements to a by-pass flow around a corked clot than of the flow measurements, which can indicate significantly reduced flow rate when resulting from by-pass flow. Of course, a moving clot also results in some, but reduced flow rate. The time dependence of the pressure and flow rate values can provide useful information since time to clear the catheter at various rates can provide a reference point.

If pulsed aspiration is desired, the status of the clot obtained from the sensors can help to guide the aspiration pulsing, as indicated in FIG. 39. An evaluation of the flow rate 1212 can involve determining if the flow rate is greater than a selected value (A) for a selected period of time (B). If yes, then this indicates that the clot has likely been cleared. Aspiration generally is terminated within a few seconds of detecting near open flow, such as 90 to 95%, of full open flow to reduce blood loss and to avoid exposing the vessel to high aspiration. Nevertheless, clearing of the clot can be verified by visually or automatically determining if the clot is in the filter, which may take place with aspiration paused. If yes that the clot is in the filter, the procedure is terminated by closing the valve or turning off the pump, and optionally verifying open flow using imaging. The catheter is removed from the patient once it is determined that blood flow has been restored. If the clot is not found in the filter, the flow and/or pressure can be checked again 1210.

If the flow is below values indicating clot clearance, then, the hardness of the clot can be estimated 1214 from the flow rate properties and/or from external measurement input into the controller. As noted above, for example, imaging can be used to evaluate clot hardness. In some embodiments, flow rates below a specified value can be used to trigger aspiration, such as below about 55%, 50%, 45%, 40%, 35%, 30%, 25% or less of maximum. If it is determined that the clot is likely hard, then an alternative process path is followed. If it is determined that the clot is likely soft, then the flow rate can be evaluated 1216 to determine if the flow rate is low enough to indicate that pulsed aspiration is desired. If flow rate is above a threshold, such as about 60%, 65% or 70%, then continuous aspiration is maintained, and the flow rate and/or pressure are continued to be monitored 1210. If pulsed aspiration is selected 1218, the pulsing can be performed by closing and opening the valve. In a basic version of this, the valve is opened and closed for selected periods of time for a selected number of times. Various pulsing options are discussed in more detail and can be considered within box 1218 of FIG. 39. If pulse aspiration is applied, the pulse can be applied 1, 2, 3, 4, 5, 6, 7, 8, 9, 10 or more than ten times prior to checking the flow at 1220. For each pulse, the valve is closed to a first period of time and opened for a second period of time (or remains open for the last pulse of a cycle). While the first period of time (closed valve) can be equal or greater than the second period of time, it has been found in some embodiments to be desirable for the first period of time to be shorted than the second period of time. In some embodiments, the first period of time is no more than ⅔, ½, ⅖, ⅓, ¼ or ⅕, of the second period of time. The first period of time can be from about 0.1, 0.2, 0.25, 0.3, 0.4, 0.5, 0.6, 0.7, 0.8, or 1.0 s to about 1.0, 2.0, 3.0, 4.0, or 5.0 s, and the second period of time can be from about 0.5, 1.0, 1.5, 2.0, 2.5, 3.0 s to about 2.0, 3.0, 4.0, 5.0, 7.0, 8.5, 10.0, or 15.0 s. In some embodiments, if the pressure differential is considered, P_{diff}=P_{flow}− Pₚᵤₘₚ, where P_{flow} is the pressure at free flowing fluid, Pₚᵤₘₚ is the pressure with no flow, pulsing can be applied when measured P over a period of time from about 1 s to about 30 s is less than (more negative) P_{flow}− 0.4 P_{diff} or 1.2 P_{flow}. With respect to flow, pulsing can be used when flow rate if from about 10% to about 65% of open flow rate. A person of ordinary skill in the art will recognize that additional ranges of pressure and flow rate cutoffs and times within the explicit ranges above are contemplated and are within the present disclosure.

With an adjustable flow valve, steady state or pulsed aspiration can be performed using intermediate decreases in flow in contrast with full opening or closing. To reduce chances of clots catching on a partially closed valves, a partially actuatable valve can be placed on the pump side of a filter/clot catcher, in some embodiments. An adjustable valve can provide considerable flexibility in programming flow limits. Also, as described above, it has been proposed to expose the catheter to a vent or other selected pressure fluid reservoir during periods where the access to the pump is closed. The vent can be at atmospheric pressure or some greater value using an appropriate fluid reservoir. Using sources of lower amounts of vacuum are also provided as an option above. Since arteries are at a pressure well above atmospheric pressure, these are all somewhat matters of different degree, especially if there is any flow being observed from the vessel. Whether or not some type of venting is used can be selected as desired. Use of another value can be timed to simultaneously open the vent when the valve to the pump is closed.

Intermittently or at the end of pulsed aspiration delivery, flow rate can be measured and evaluated 1220. The flow rate can be evaluated based on a threshold value and/or an evaluation of the change in flow. With respect to the flow rate, the threshold (C in FIG. 39) of the flow rate to continue applying pulsed aspiration can be selected, for example, to be less than about 45%, 50%, 55%, 60% or 65% of the full flow rate value, and above which continuous aspiration is resumed at 1210. In some alternative embodiments, the review of flow at 1220 can be skipped, and the process can go directly to evaluation at 1210 once a pulsing cycle is completed. A person of ordinary skill in the art will recognize that additional ranges of flow rate cutoffs within the explicit ranges above are contemplated and are within the present disclosure. To evaluate the change in flow rate with significant noise in the time dependence, the flow as a function of time can be fit to a curve using known algorithms. For example, a polynomial fit can be used, although any suitable curve fitting procedure can be used. Once a curve is fit to the flow, the slope provides information on the change in flow rate as a function of time with interference from the noise removed, and if the flow rate is increasing significantly, continuous aspiration can be used, and the procedure reverts to checking of the pressure and flow rate at 1210. If the flow rate is insufficient and/or if the increase in flow rate over time is insufficient, pulsed aspiration can be continued 1218. Once pulsed aspiration is discontinued, checks for clot clearance is continued 1212.

If the clot is determined likely not to be soft, high frequency pulsing can be applied 1230, although the soft clot procedures can still be followed if desired. For this pulsing the frequency can be at least about 15 Hz, 20 Hz, 30 Hz, 50 HZ or from about 25 Hz to about 200 Hz. A person of ordinary skill in the art will recognize that additional ranges of frequency within the explicit ranges above are contemplated and are within the present disclosure. High frequency pulsing is generally, but not necessarily, applied with symmetric pulses, equal times open and closed. After a selected period of time, flow rate and/or pressure is evaluated 1232, and evaluation of the flow rate readings 1234 can be used to determine whether or not the flow rate is increasing. This can again be performed by fitting the flow rate curve to remove the noise and looking at the slope. If the flow rate is increasing, the value of the flow rate can be evaluated 1236. If the value of the flow rate is below a threshold, then high frequency pulsing 1230 can be continued. If the flow rate is above a threshold, continuous aspiration can be used and the clot status can be evaluated form the flow rate and/or pressure readings 1210. If the flow rate is not increasing 1234 after applying high frequency pulsing 1230, a practitioner may determine that additional treatment devices should be delivered 1238 to assist with clot removal.

Once the catheter is placed into the patient and the clot is cleared, the flow should return to the open flow pressure and flow measurements based on the calibration, which may involve small adjustment for blood versus biocompatible priming fluid, such as saline. Transient readings can cause potential mistaken conclusions, but this can be accounted for, as suggested in the bench studies reported below. With respect to further elaboration on evaluation of clot clearance under 1212, pressure and/or flow rates can be used as follows. Once the pressure is within about ±10%, ±8%, ±7%, ±6%, ±5%, from the 100% pressure value (open flow baseline) for more than a specific amount of time (such as 0.1-20 seconds (s), 0.5-10 s or 1-5 s)), it can be assumed that the clot has been captured. Similarly, once the flow rate is greater than a cutoff portion, such as about 90%, 92%, 94% 95%, 96%, 97%, of the open flow rate baseline (100% value) for a specified period of time (such as 0.1-20 s, 0.5-10 s or 1-5 s), this is indicative of clot capture. Also, the slope in flow as a function of time will go to approximately zero. Clot capture can be confirmed through visual determination of the clot being in the filter and/or through sensor measurement of the clot in the filter. But if clot fragmentation occurs, then determination of clot material in the filter can be inconclusive on its own. Consistent results with flow and/or pressure sensor readings and determination of clot material in the filter, provides appropriate verification of clot clearance. Once clot capture has been determined 1240, the valve can be closed and consideration can be directed to ending the procedure 1242 and/or performing any additional tasks. Valve closure can be performed automatically to provide to faster response time and allowing the health care professional to attend to other considerations at the same time. A person of ordinary skill in the art will recognize that additional ranges of pressure and flow rate cutoffs within the explicit ranges above are contemplated and are within the present disclosure.

As noted above, the measurements can be used to evaluate clot hardness in a relative sense relating to response to aspiration. This flow based clot evaluation does not necessarily correlate with a conventional hardness measurement, but it does presumably depend on the mechanical properties of the clot, such as hardness and elasticity. The flow based clot hardness determination may be supplemented or replaced with alternative means to evaluate the clot hardness. A soft clot is observed to result in a more rapid pressure drop after the aspiration is initiated at the catheter relative to the pressure drop observed for a harder clot. Corresponding plots are presented below from bench studies. While not wanting to be limited by theory, this observation would seem consistent with the clot being more effectively brought into the catheter lumen where it blocks the flow. A soft clot though then continues to respond to the pressure, and within about two seconds or less flow begins to increase as the clot is ingested into the catheter and gradually moves down the catheter. For harder clots, the pressure drops more slowly since the clot is deformed less by the aspiration, but then the pressure continues to drop. If kept under constant aspiration, the clot would either be ingested into the catheter and gradually be pulled down the catheter or it remains stuck at the catheter tip. A rapid, greater than 10 hertz, pulsed aspiration can be applied to try to either uncork the clot at the catheter tip or fragment the clot so that the clot can be cleared. The sensor reading can evaluate the efficacy of the rapid aspiration pulsing on the clot, and if no effective change in the clot status in a selected period of time, generally from 10 seconds to 5 minutes, a further treatment tool can be delivered to facilitate clot removal.

With respect to measuring flow rate and pressure measurements in the various steps of FIG. 39, the measurements can be continuous on the time frame of the processor clock monitoring the sensors, but evaluation of the flow rate and/or pressure to influence adjustments in the aspiration can be set according to prescribed timings built into the procedure. After aspiration is started, such as by opening the automatic valve, the flow rate and pressure can be monitored at short time intervals which are effectively continuous. The status of the flow rate can be determined at a selected time point, such as 1 second, for evaluating process status. In the context of FIG. 39, the pulsed aspiration of a soft clot is presented as continuous pulsing at a select frequency for a select period of time. Various options for varying this pulsing performance is described next.

As noted above, for clots that are not soft, higher frequency pulsing is applied with the objective of breaking up or wakening the clot structure to provide for conforming the clot for entry into the catheter tip. The frequency can be selected from about 10 hertz to the response frequency of the valve and corresponding electronics., such as about 200 hertz. For soft clots, the pulsed aspiration is found to be more effective when the time period of the closed valve is shorter than the time period of the open valve. So the valve can be open for 2 times to 25 times the length of time that the valve is closed. The overall period of repeating the open and closing of the valve can be from about 0.025 seconds to about 25 seconds. A person of ordinary skill in the art will recognize that additional ranges within these explicit ranges of frequencies and period are contemplated and are within the present disclosure.

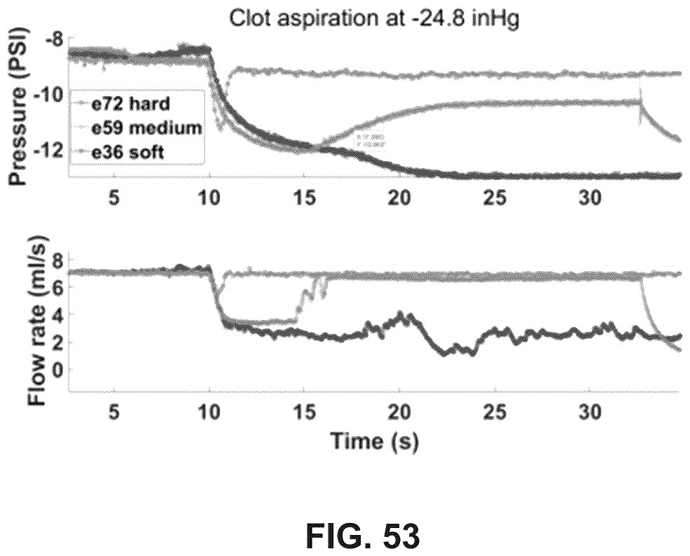

When aspiration is being applied, the clot status can be checked at prescribed intervals, whether the aspiration is continuous or pulsed. When the aspiration is pulsed, various approaches can be used with respect to timing to check the aspiration status. In some embodiment, the status can be checked after every pulse cycle, optionally with a delay, such as one second, and the pulsed aspiration can be continued or continuous aspiration can proceed, as described in FIG. 53. Thus, the triggering of pulsed aspiration is reevaluated after each pulse. In other embodiments, the pulsed aspiration can be continued for multiple pulses, such as two, three, four, five, or more pulses, before the clot status is evaluated. In further embodiments, after one or more pulses, the flow is just checked if the flow is above a threshold value, and if not, another pulse is applied, and if it is, then constant aspiration is continued with corresponding periodic evaluation of clot status. Thus, in this alternative pathway, multiple aspiration pulse cycles can be completed without checking for clot clearance such that the pulsing can continue on a shorter time frame.

Lab Bench Testing

Two sets of measurements are described, which use slightly different system configurations. The first set of experiments evaluates the catheter performance for MIVI Q-Catheter™ with a distal access aspiration catheter under continuous aspiration with comparisons to performance from competing full length catheters. Significantly better performance is observed with the Q-Catheters™. The second set of experiments evaluates the effects of pulsed aspiration.

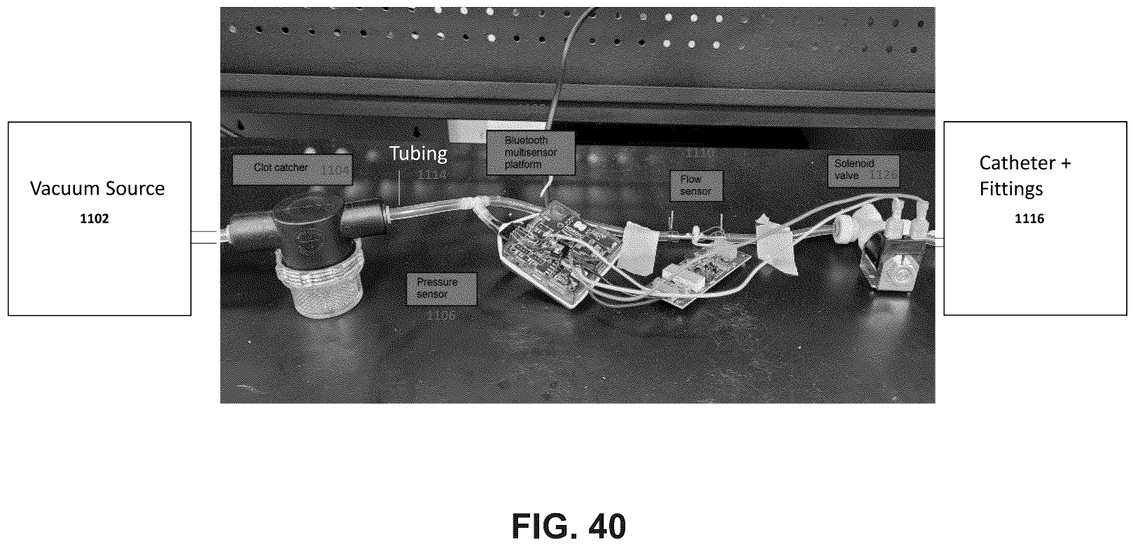

A first experimental system configured to allow for real time monitoring of a simulated aspiration thrombectomy that was used in the trials described below is illustrated in FIG. 40. The system generally included a vacuum source 1102, a clot catcher (filter) 1104, a pressure sensor 1106, controller 1108, a flow sensor 1110, a valve 1112, extension tubing 1114 and aspiration catheter 1116.

The vacuum source 1102 was a vacuum pump configured to operate at a pressure of about −30 inHg. The vacuum source 1102 was fluidly connected to the catheter 1118 by extension tubing 1114 and valve 1112. The pressure sensor 1106 was fluidly connected between the vacuum source 1102 and catheter 1118 and configured to measure real time pressure of the fluid passing through the extension tubing 1114. The measurements were communicated to controller 1108.

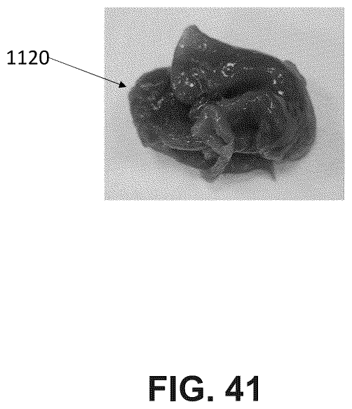

Filter 1104 was fluidly connected between the vacuum source 1102 and pressure sensor 1106 and sized appropriately to capture simulated clots while letting fluid flow essentially unimpeded. The clot 1120 used to simulate a red blood cell clot is generally shown in FIG. 41 and was a soft Thrombotech™ synthetic clot obtained from Biomedix. The clot was sized to have a diameter of about 2.0 mm.

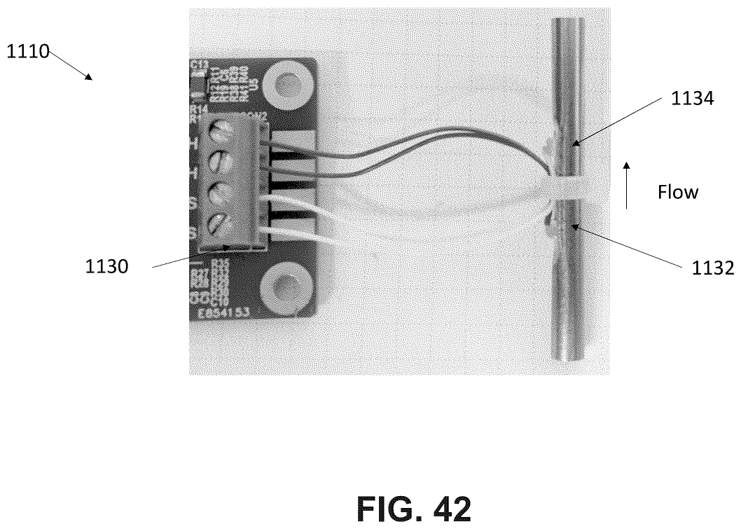

A close-up of the flow sensor 1110 is illustrated in FIG. 42 and included a printed circuit board 1130 electrically connected to unheated 1132 and heated 1134 temperature sensors. The printed circuit board 1130 was configured to measure the real time flow rate using the constant temperature anemometer principle (i.e., the amount of heat removed from the heated temperature sensor 1134 by a flowing fluid is related to that fluid's velocity with the unheated temperature sensor 1132 being used to compensate for variations in the air temperature). The measurements were communicated to controller 1108 by an electrical wire.

Valve 1126 was a solenoid valve electronically connected to controller 1108 and configured to open and close based on signals sent from controller 1108. The controller 1108 was configured to receive input regarding measurements from pressure sensor 1106 and flow sensor 1110 and communicate output to a display and to the valve 1126 for the purpose of opening and closing the valve. Tubing running through solenoid valve 1126 to pump 1102 connects to catheter+fittings 1116.

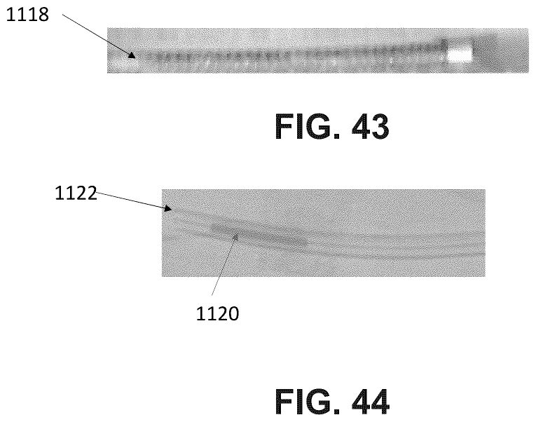

Catheter is generally illustrated at its distal tip 1118 in FIG. 43 and, as shown, included a Q™ 4 catheter (from MIVI Neoroscience, Inc.) having an OD of 1.4 mm, ID of 1.09 mm and which is generally described above. In some measurements, other sizes of Q™ catheters were used, including a Q6 catheters. For comparison purposes, a Zoom45 catheter (from Imperative Care Inc.) having an OD of 1.52 mm and a 4Max catheter (from Penumbra Inc.) having an OD of 1.42 mm were also used. Silicone tubing 122 (shown in Figure PE), having an ID of 1.5 mm or 1.6 mm was used to simulate a blood vessel.

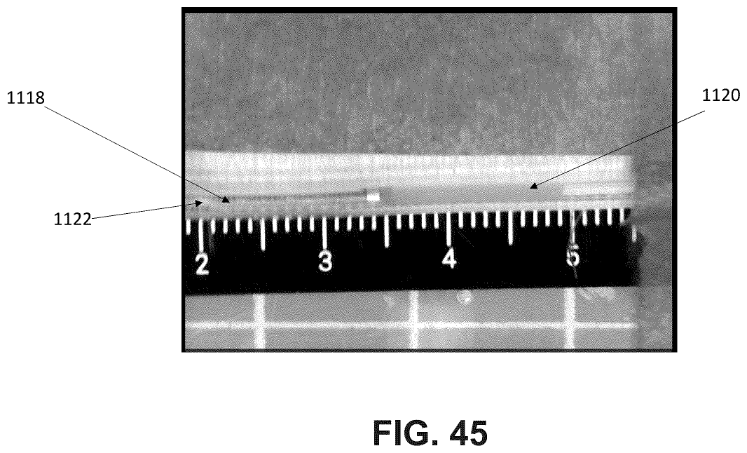

For each trial, a soft Thrombotech™ synthetic clot 1120 was cut to have a length of about 10 mm and positioned in the silicone tubing 1122 using a syringe as illustrated in FIG. 44. The solenoid valve was electronically closed by the controller. To obtain baseline pressure and flow rate values, the vacuum pump was turned on for approximately 1 minute during which time the pressure within the extension tubing was continuously measured by the pressure sensor to obtain the maximum negative pressure Pc available for the pump (the flow rate Fc being equal to 0). The tip of the catheter was then immersed in saline and the solenoid valve was electronically opened to fill the system with saline. Some trials were also performed in glycerin 40% to mimic the performance with blood. The pressure and flow rate within the extension tubing was then measured by the pressure sensor and flow sensor to obtain an open control pressure Pₒ and steady state flow rate Fₒ of saline through the unconstrained catheter, which is the effective 100% flow rate value. The solenoid valve was then electronically closed for approximately 10 seconds to remove any air within the system. The catheter was then passed through a hemostatic valve and its distal end directed to the clot face within the silicone tubing (see FIG. 45). Once the catheter was in place, the solenoid valve was opened and the pressure and flow rate within the extension tubing was continuously measured by the pressure sensor and flow sensor.

Bench testing can also be used to correlate flow and pressure measurements with the system, using a selected catheter size, and corresponding measurements with blood or a blood analogue, such as aqueous glycerin 40%. Corresponding measurements can be made for free flow and no flow limits with saline and with blood/blood analogue to form a look up correlation table or fit correlation equations. If desired, the look up table or correlation equations can be programmed into the controller to allow for determining flow and pressure limits during system preparations at the start of a procedure, as described above.

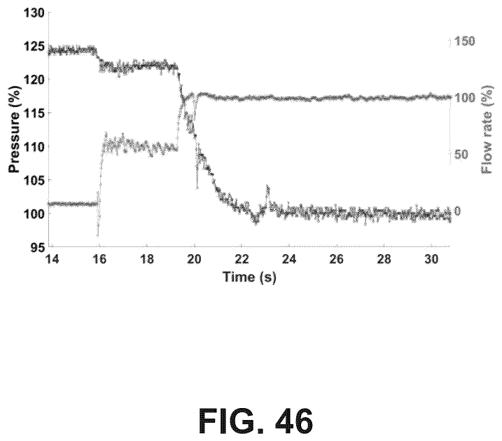

The pressure and flow rate measurements for an exemplary trial are shown in FIG. 46. In this trial, the maximum negative pressure Pc was about −29 inHg (125%) and the open control pressure was about −22 inHg (100%) for the Q4 catheter. After the solenoid valve was opened at 10 seconds, the negative pressure increased to about −27 inHg (120%) and remained steady while the flow rate varied between about 50-60% of Fₒ for approximately 9 seconds suggesting the clot was corked. The negative pressure and flow rate then increased to Pₒ(100%) and Fₒ(100%) and remained steady suggesting the clot was travelling through and clearing from the system

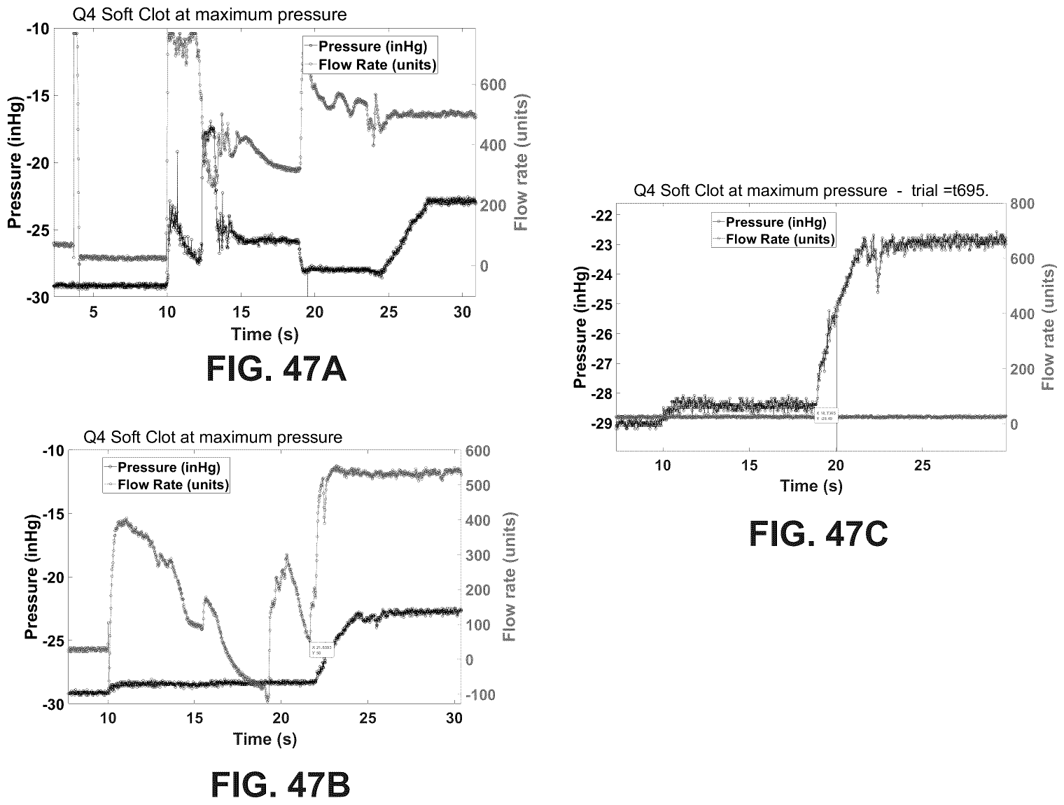

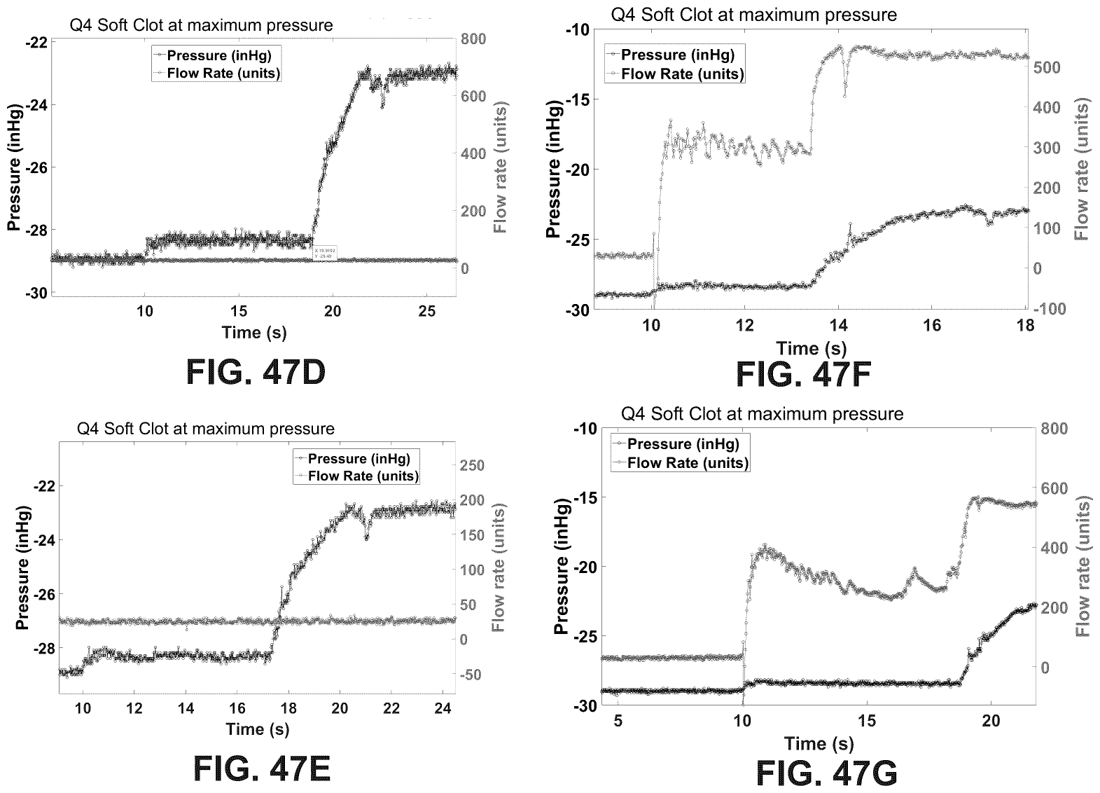

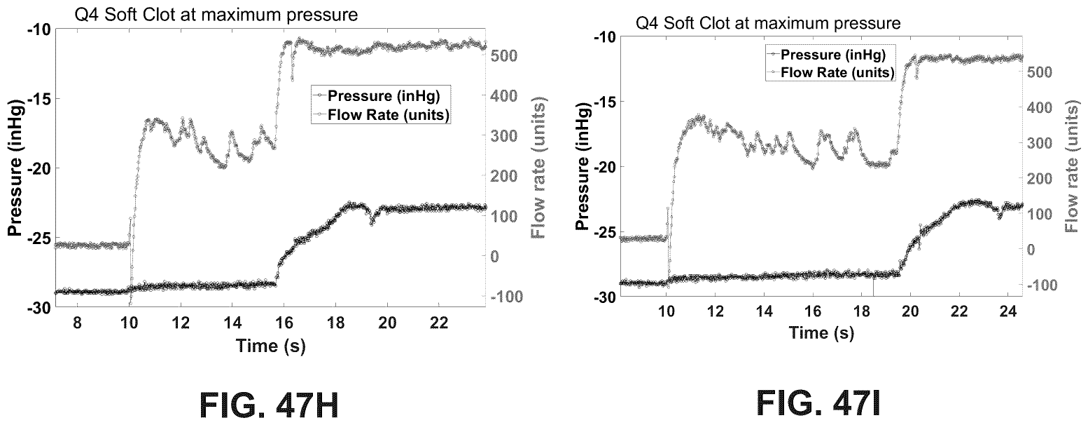

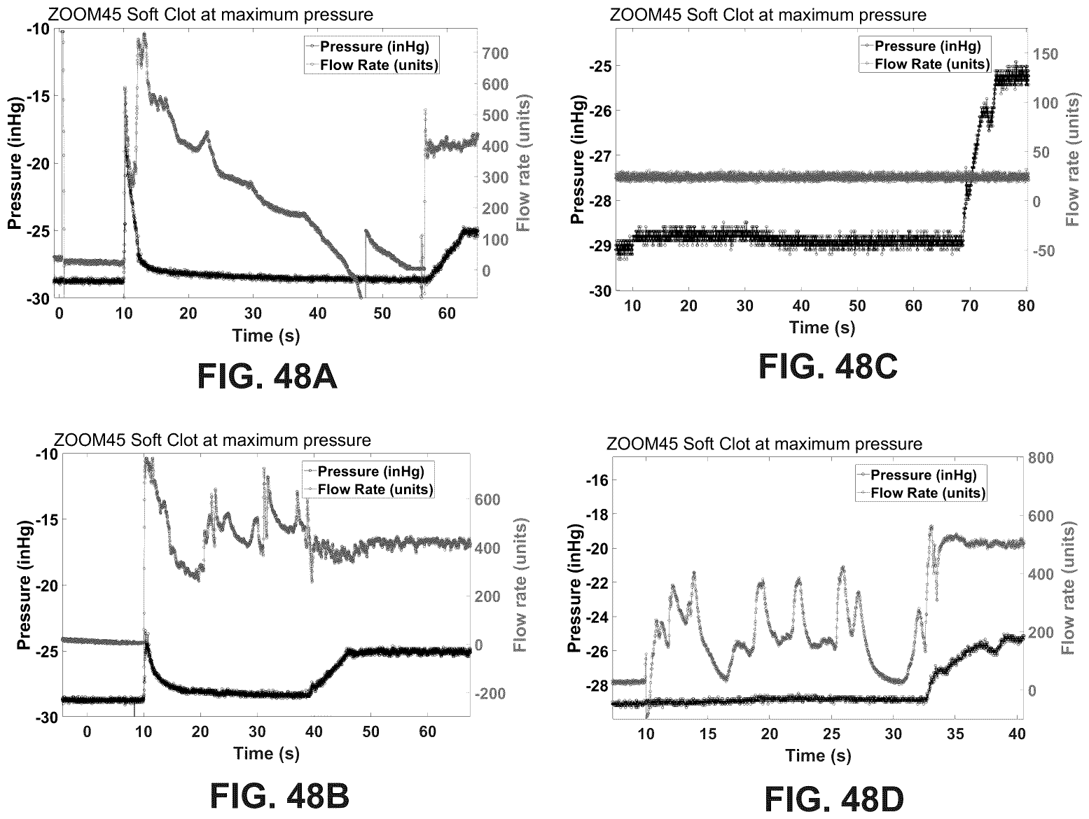

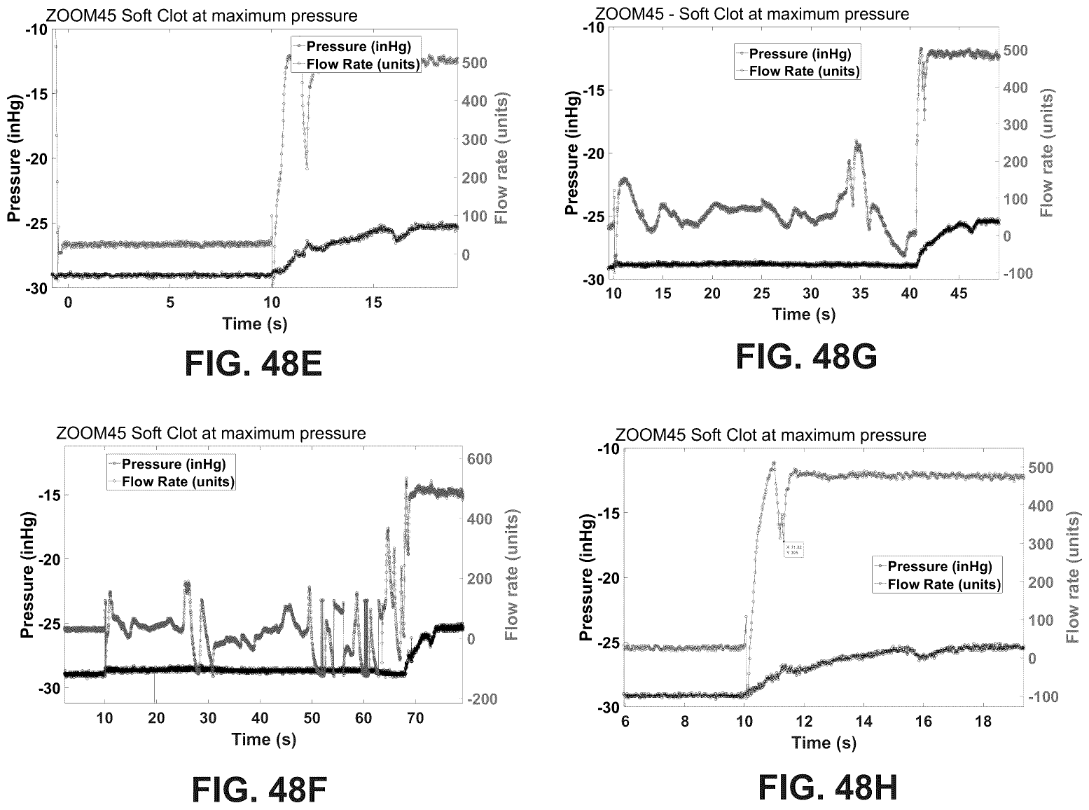

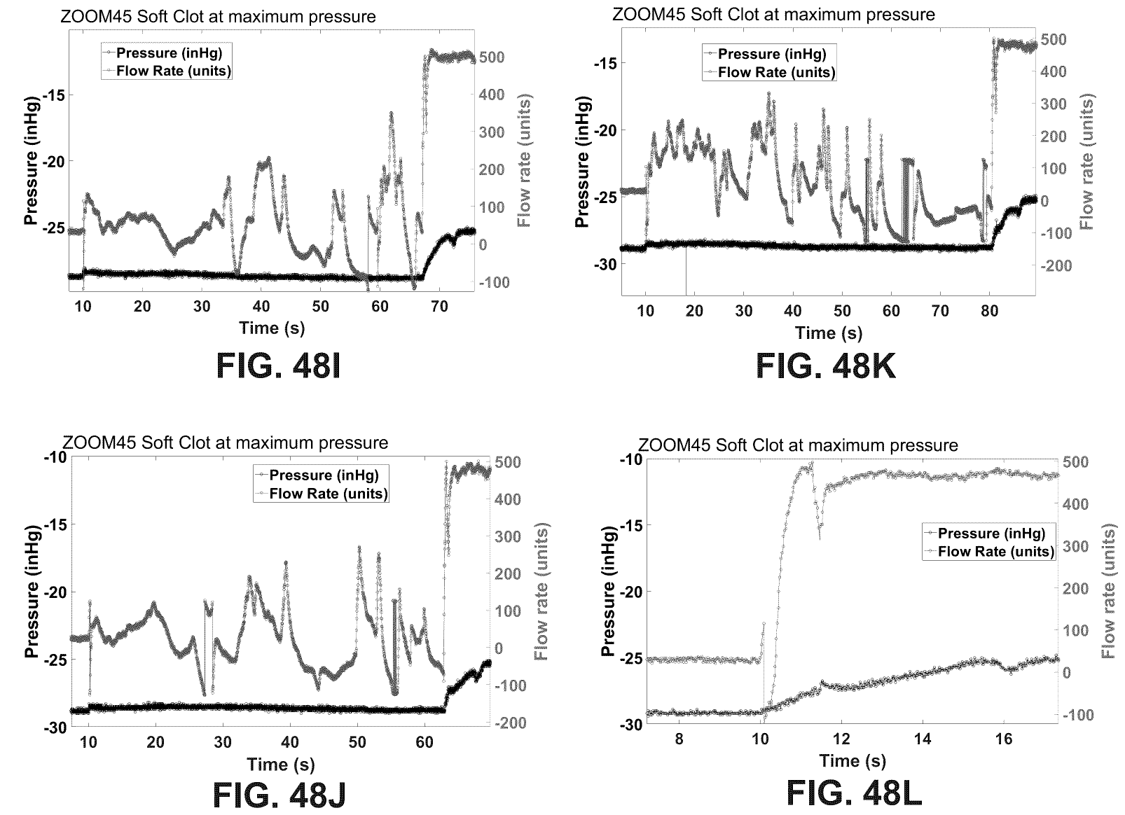

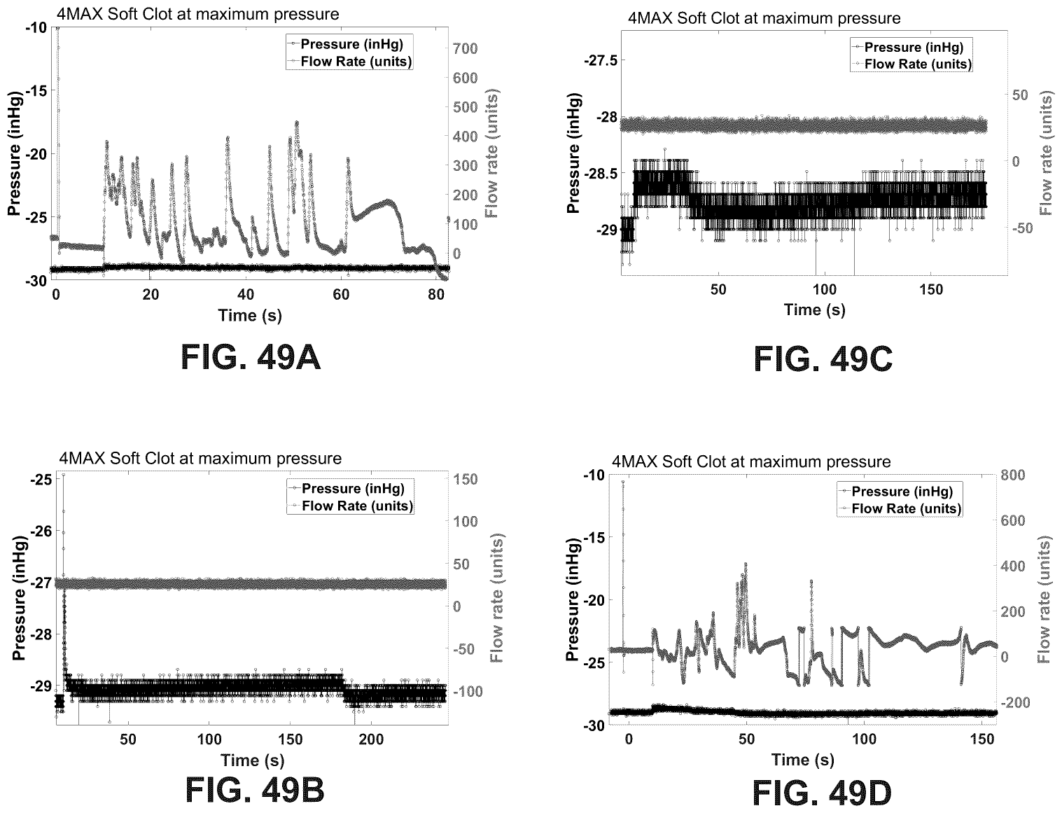

The results of the trials are summarized below in Tables 2, 3 and 4 and illustrated in FIGS. 47A-I, 48A-L, and 49A-D:

TABLE 2

| | | | Clot | | | |
|---|---|---|---|---|---|---|
| | | | length | | | |
| | | | inside | | Smart | |
| | | Clot | silicone | | tube | Catheter |
| Trial | Catheter | length | tube | | ingestion | ingestion |
| # | Type | (mm) | (mm) | Notes | (secs) | time |
| 1 | Q4 | 10.00 | 18 | | 14.6 | NA |
| 2 | Q4 | 10.00 | 15 | | 11.6 | 2.6 |
| 3 | Q4 | 10.00 | 16 | | 8.0 | 1.8 |
| 4 | Q4 | 10.00 | 16 | | 8.7 | 1.6 |
| 5 | Q4 | 10.00 | 16 | | 8.9 | 2.6 |
| 6 | Q4 | 10.00 | 15 | | 7.3 | 0.8 |
| 7 | Q4 | 10.00 | 16 | 16 OR | 3.4 | 0.9 |
| | | | | 16.5 mm | | |
| 8 | Q4 | 10.00 | 16 | | 8.6 | 1.8 |
| 9 | Q4 | 10.00 | 16 | Signal did | NA | 1.8 |
| | | | | not save | | |
| 10 | Q4 | 10.00 | 16 | | 5.6 | 0.6 |
| 11 | Q4 | 10.00 | 16 | | 12.8 | 3.6 |
| 12 | Q4 | 10.00 | 15 | | 9.4 | 1.9 |

TABLE 3

| | | | Clot | | | |
|---|---|---|---|---|---|---|
| | | | length | | | |
| | | | inside | | Smart | |
| | | Clot | silicone | | tubing | |
| Trial | Catheter | length | tube | | ingestion | Catheter |
| # | Type | (mm) | (mm) | NOTES | (secs) | ingestion |
| 1 | ZOOM45 | 10.00 | 16 | | 47.3 | 2.8 |
| 2 | ZOOM45 | 10.00 | 15 | | 29.1 | 2.3 |
| 3 | ZOOM45 | 10.00 | 16 | | 58.5 | 1.6 |
| 4 | ZOOM45 | 10.00 | 16.5 | 16.5(17 | 22.6 | 1.4 |
| | | | | mm) | | |
| 5 | ZOOM45 | 10.00 | 15 | | 1.7 | 0.4 |
| 6 | ZOOM45 | 10.00 | 18 | | 57.8 | 1.5 |
| 7 | ZOOM45 | 10.00 | 16 | | 31.5 | 0.8 |
| 8 | ZOOM45 | 10.00 | 16 | | 1.3 | 0.3 |
| 9 | ZOOM45 | 10.00 | 16 | | 57.1 | 1.5 |
| 10 | ZOOM45 | 10.00 | 16.5 | 16 OR | 52.8 | 3.7 |
| | | | | 16.5 mm | | |
| 11 | ZOOM45 | 10.00 | 16 | | 70.4 | 0.7 |
| 12 | ZOOM45 | 10.00 | 15 | | 1.5 | 0.5 |

TABLE 4

| | | | Clot | Corked | | |
|---|---|---|---|---|---|---|
| | | | length | (Y = | SMART | |
| | | Clot | inside | YES, | tubing | Catheter |
| TRIAL | Catheter | length | silicone | N = | ingestion | ingestion |
| # | TYPE | (mm) | tube (mm) | NO) | (secs) | (secs) |
| 690 | 4MAX | 10.00 | 16 | Y | >150 | 3.3 |
| 693 | 4MAX | 10.00 | 17 | Y | >160 | 9.0 |
| 698 | 4MAX | 10.00 | 15 | Y | >230 | 12.7 |
| 701 | 4MAX | 10.00 | 16 | Y | >150 | 6.4 |
| 703 | 4MAX | 10.00 | 15 | Y | >130 | 4.3 |

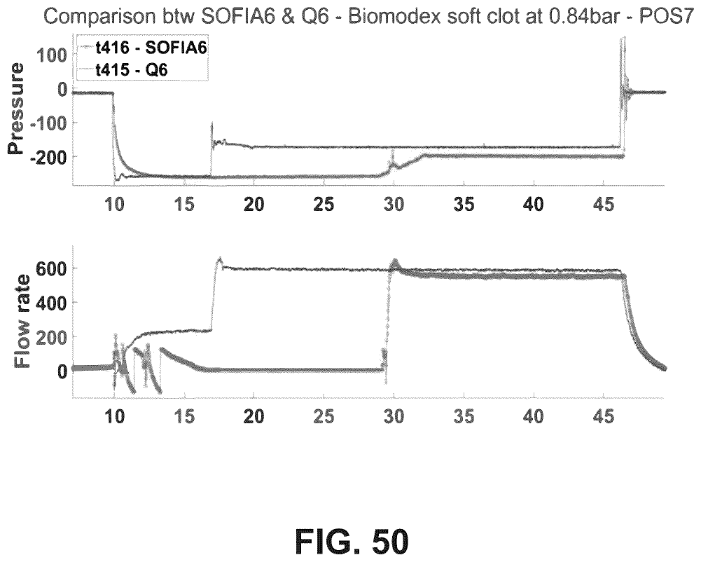

The first trials for each catheter were run on the experimental system described above whereas the solenoid valve was removed from the system for the remaining trials. This flow rate sensor was then replaced with a different flow rate sensor for most of the trials, but the results did not seem effected. The comparison of results with a Q6 catheter and a Sophia6 catheter are shown in FIG. 50.

The controller can be configured to compare the pressure and flow rate curves with information stored in a memory module to identify the aspiration stages. The procedures described herein can be programmed into the controller to make the comparison and send a signal to the solenoid valve to close the valve when the clot has cleared the system (and/or open and close the valve during pulsing when the clot is corked). For example, the controller can be programmed to maintain the solenoid valve in an open position (or pulse mode) when the pressure is greater than 110% (the clot is corked) and close the solenoid valve (or turn the vacuum pump off) to avoid blood vessel damage when the pressure is between 95-105% after the clot has cleared system and is captured in the filter. Regarding flow rate, the controller can be programmed to maintain the solenoid valve in open position (or in a pulse mode) when the flow rate is less then 90% of the open flow rate Fₒ for at least 10 seconds (the clot is corked) and then close the solenoid valve (or turn the vacuum pump off) to avoid blood vessel damage when the flow rate is greater than 90% of Fₒ for an appropriate period of time, such as at least 10 seconds.

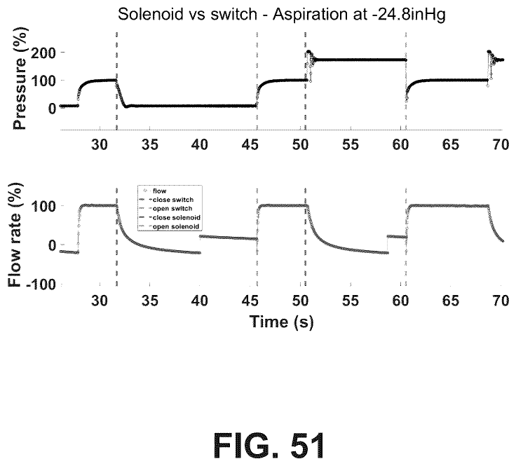

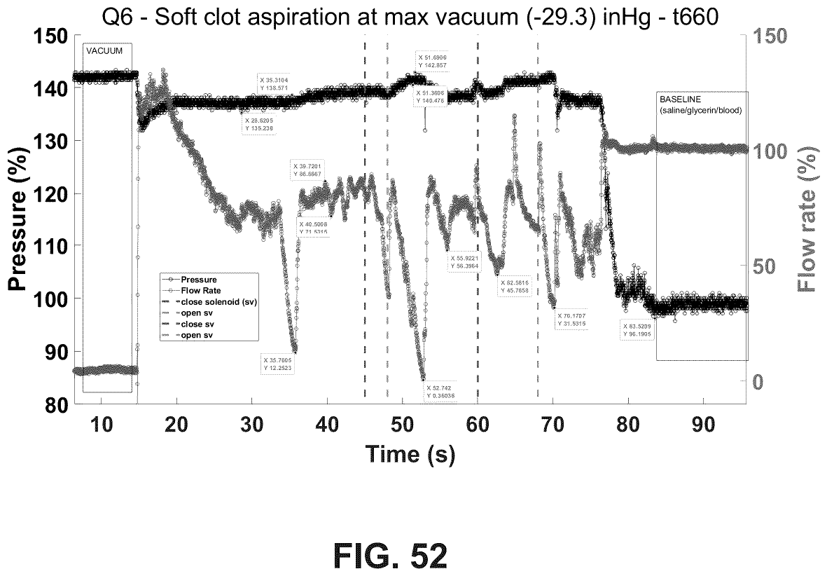

FIG. 51 shows flow and pressure measurements where the valve is closed and reopened at selected increments shown with verticle dashed lines. When the valve is closed, the flow gradually diminishes over several seconds and the pressure responses with a somewhat shorter transient. Trials with a simulated soft clot with aspiration pulsed are shown in FIG. 52.

Some trials were also performed using medium hardness clots and hard clots. Representative pressure and flow measurements are compared in FIG. 53 plotted in superimposed format. The medium and hard clots were particularly challenging since they were formed with a larger than typical diameter for the tubing size and compressed into the tubing. In these trials, the soft clots cleared rapidly, suggesting that the clots did not cork. The medium clot cleared the catheter in 6 or 7 seconds. For the medium clot, the pressure did not recover to the baseline pressure corresponding to free flow. An examination of the catheter tip following clot clearance indicated that the polymer at the very tip was damaged which is consistent with the observed pressure after clot clearance and not unexpected based on the excess diameter of the clot samples. Nevertheless, the catheter did succeed in clearing the clots. The flow also does not quite recover, but the difference is significantly smaller. For the hard clot, it was not captured in the plotted time frame. Looking out to longer times, the hard clot was captured in about 45-50 seconds. Again, for the hard clot, the pressure did not return to the free flow baseline and the catheter tip was folded and damaged.

A further set of bench testing was performed to examine the effects of pulsed aspiration on soft clots. For these experiments, the solenoid valve was placed between the filter and the pump, as shown in FIG. 40. The soft clot model of FIG. 41 was used to evaluate clot ingestion into the catheter and into the system after clearing the catheter. For these studies, a MIVI Q4™ or Q6™ aspiration catheter was used with a MIVI™ guide catheter.

In a first set of experiments, constant aspiration was compared with pulsed aspiration without any evaluation of flow to gate the aspiration delivery. The pulsed aspiration was selected to have varying amounts of time with the valve open and the valve closed. The tests are indicated in Table 5.

TABLE 5
19 Trials Total
8 Constant Method Trials
11 Pulsation Trials

| | |
|---|---|
| 3 Method A Trials | 0.1 sec ON + .01 sec OFF |
| 3 Method U Trails | 5 sec ON + 0.1 sec OFF |
| 5 Method Z Trials | 5 sec ON + 0.5 sec OF |

Of the pulsed trials, 8 had longer periods of the valve open (aspiration on) relative to the time that the valve was closed (aspiration off), and 3 trials with equal periods of the valve open and closed.

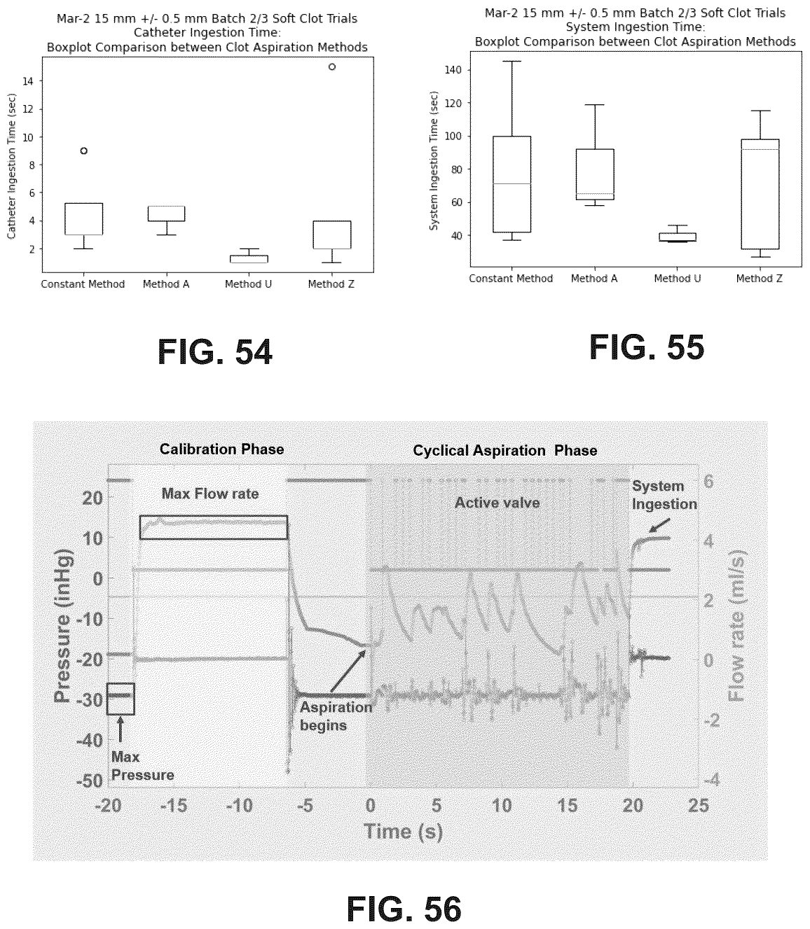

Box plots of the catheter ingestion time (clot within catheter) and the system ingestion time (clot clears catheter) are plotted in FIGS. 54 and 55. In general, the 0.1 seconds (s) off-0.1 s on results performed roughly comparably to the constant aspiration results. Trials with pulsed aspiration with 5 s on and 0.5 s off performed worse than constant aspiration. The best performance both with respect to median times and spread was obtained with aspiration with 5 s on and 0.1 s off.

Another set of experiments was performed to evaluate selective application of pulsed aspiration based on flow measurements. Experiments were performed with either constant aspiration or with aspiration delivered according to the various procedures outlined above. For these tests, six sequential flow data points were used to evaluate the desirability of using aspiration as well as evaluating clot status. Four specific procedures were tested. In all of the procedures, the flow sensor was zeroed at the beginning of the procedure, and the catheter was calibrated with open valve flow and closed valved flow to get the ranges of flow and pressure measurements. Then, the testing was started. Again, for all of the versions of the procedure, an initial flow evaluation checked to see if flow was within a certain percent (>95%) of the full calibrated flow, which indicates that the clot has cleared to catheter. If the flow is at least this amount, the procedure is stopped since the clot is cleared. Following this initial flow evaluation, the four procedures varied form each other. In these procedures, the clot hardness was not tested. Effects of clot hardness on flow is discussed below.

In a first procedure, if the flow is below the value indicating clot clearance, then the flow is evaluated to determine if pulsed flow is desired. If the flow is above a set percent of the full flow, such as 65%, then, continuous aspiration is continued. If the flow is below the threshold value, then an aspiration pulse is applied, by closing the valve for the specified time and then reopened. After reopening the valve and a one second delay, flow is checked and the procedure loops back to the constant aspiration track where clot clearance is checked, and the desire to applied an aspiration pulse is similarly reevaluated. In the continuous aspiration track, the flow is checked periodically to see if the clot is cleared and if not if continuous aspiration should be applied or if an aspiration pulse should be applied. In this procedure, aspiration can have periods of constant application with intermittent periods of pulsed aspiration.

A plot of flow and pressure during this first procedure is shown in FIG. 56. After a calibration phase with the valve open, the valve is closed and the flow gradually diminishes to zero flow. Then, the valve is opened to initiate aspiration. In this test, pulsed aspiration, i.e., closing the valve, occurs multiple times at varying time intervals according to flow measurements. The closing of the valve is noted by the upper plot. The clot is cleared in about 20 seconds.

In a second track, the procedure continues similarly to the first procedure through checking to see if an aspiration pulse should be delivered. If an aspiration pulse is delivered, then the option of providing a further aspiration pulse is evaluated prior to looping back to the continuous aspiration track. Thus, after reopening the valve, a delay period, one second used, is waited, and then the flow is evaluated again to determine if flow is below the threshold indicating application of an aspiration pulse. If flow is increased beyond the threshold, then continuous aspiration is applied and the procedure reverts to the continuous aspiration track. If flow has not increased above the threshold, another aspiration pulse is delivered by again closing the valve and reopening the valve after the prescribed time. In this way, a plurality of pulses can be applied without reverting to the continuous aspiration track. A third procedure is similar to the second procedure except that a plurality of aspiration pulses are delivered prior to evaluation of the flow. In the test, three aspiration pulses were applied with the valve closed and reopened three times for the prescribed times, and then the flow is again checked if a further three pulses should be delivered or if aspiration should revert to the constant aspiration procedure.

In a fourth procedure, the use of the curve fitting the flow curve is used to provide additional information on the clot status. A polynomial fit is made to the flow as a function of time. An additional pressure evaluation is also performed. In the constant aspiration path, an evaluation of the flow to determine if an aspiration pulse will be applied, of the flow is less than one value then a pulse is applied or if the flow is less than a somewhat greater value but the slope of the polynomial fit is below a certain value, then an aspiration pulse is applied. In other words, either a lower flow value or a low flow value and slow increase in flow can trigger the use of an aspiration pulse. If an aspiration pulse is applied, when the valve is closed, the pressure is checked, and if the pressure is greater than a set threshold indicating more exposure to blood pressure, then continuous aspiration is resumed. If the pressure is not above the threshold, then the valve is opened and the flow is checked to determine whether or not to apply a further aspiration pulse. If the flow is above a certain threshold or if the slope of the polynomial fit is above a set threshold, then continuous aspiration is resumed, and otherwise, another aspiration pulse is applied. This procedure is continued until continuous aspiration is resumed, and clot clearance is determined from the flow being above the prescribed value.

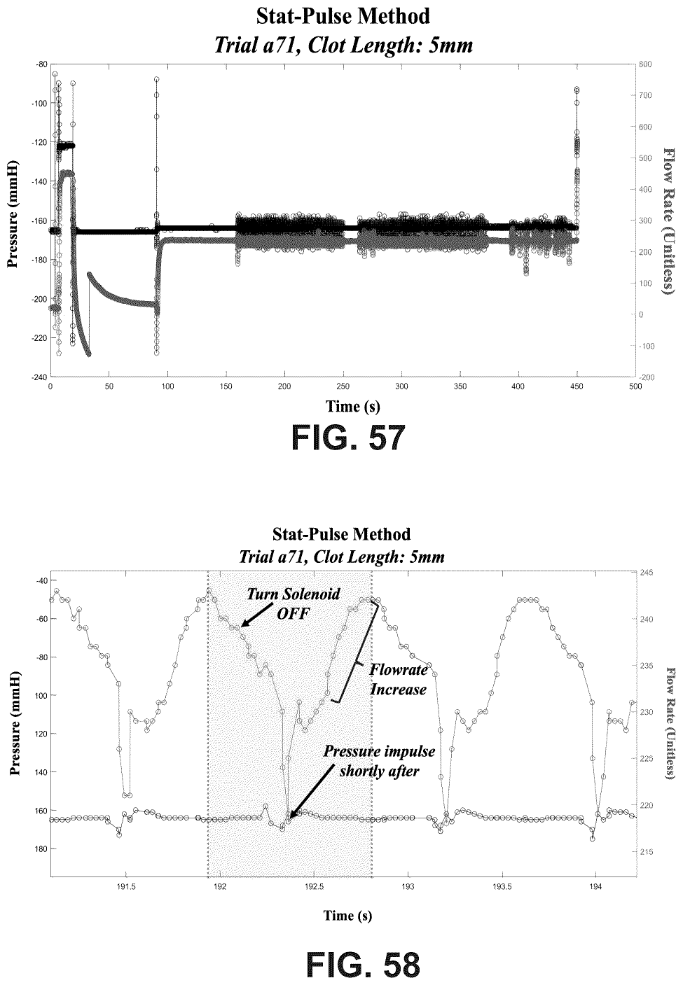

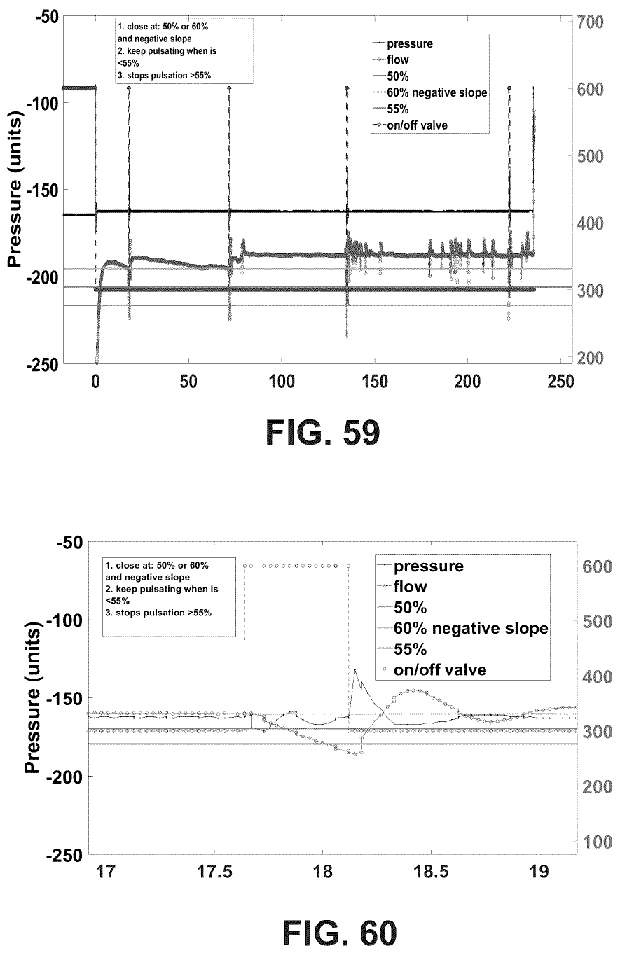

Plots of pressure and flow evaluated during a run of this fourth procedure are shown in FIGS. 57 and 58 using a clot of 5 mm length. There are three stretches of time where pulsed aspiration is applied prior to clot clearance. FIG. 58 shows an expanded section of the plot over a short time frame where four aspiration pulses are shown. FIGS. 59 and 60 depict another run using the fourth procedure. In this run, pulsed aspiration is used more intermittently. FIG. 60 shows an expanded section from 17 to 19 seconds.

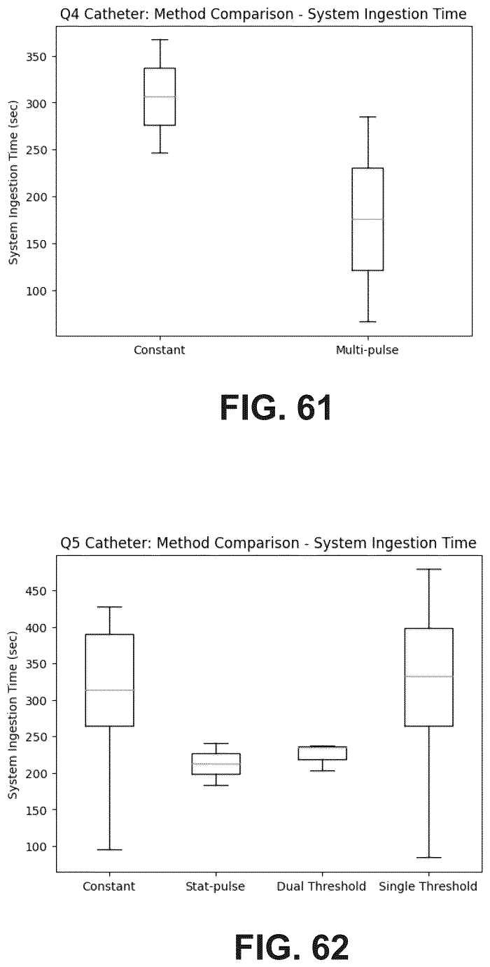

The first procedure is found to yield similar results as continuous aspiration. The other three procedures provided significantly faster clot clearance. Box plots of the results are plotted in FIG. 61 for a Q4 catheter using constant aspiration and the fourth procedure, and in FIG. 62 for a Q5 catheter with constant aspiration and the first three procedures.

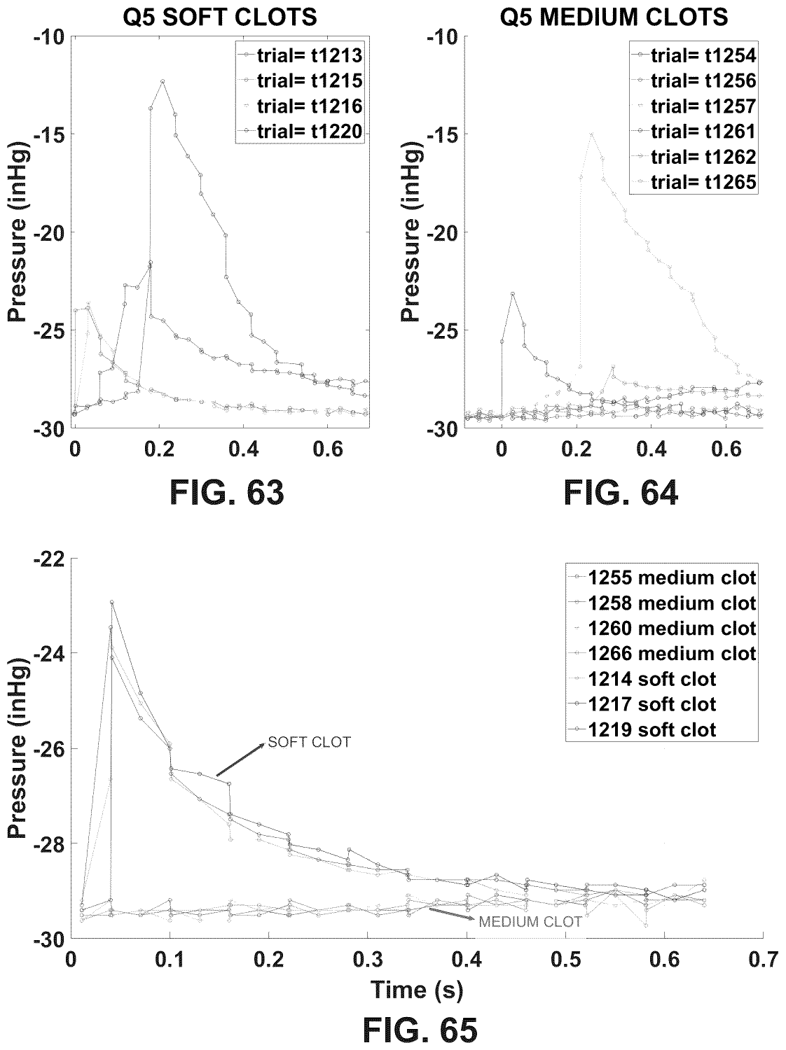

With respect to evaluation of soft versus medium plots, short time measurements of the pressure were found to also provide information on the clot properties. FIGS. 63 and 64 provide pressure measurements as a function of time at times on the order of a second using a Q4 catheter with soft clots (FIG. 63) or medium hardness clots (FIG. 64). A short time pressure spike can be observed for the soft clots and not necessarily for the medium clots. FIG. 65 is a plot of pressure as a function of time for short times on the order of a second for a full length aspiration catheter using soft or medium clots. For the full length catheter, the soft clots exhibited the pressure spike and the medium clots consistently lacked the pressure spike, which indicates the diagnostic utility of the determination of the pressure spikes and the improved aspiration ability of the Q catheters.

Further Inventive Concepts

A1. An aspiration thrombectomy system comprising:
an aspiration catheter assembly comprising a suction lumen extending from a proximal end with a connector, to a distal opening;
fittings comprising a branched manifold with a first branch comprising a hemostatic valve and a second branch comprising a connector, wherein the fittings are in fluid communication with the suction lumen of the aspiration catheter;
a pump;
a conduit connected to the pump and to the connector of the second branch;
a flow meter connected to the conduit to measure flow rate to the pump;
a filter connected to the conduit to remove clots from the flow;
a first automatic valve configured to control flow between the fittings and the suction lumen; and
a controller connected to the flow meter and the automatic valve and wherein the controller controls the valve based on measurements received from the flow meter and pulses the valve based on measured flow values.

A2. The apparatus of claim A1 further comprising a first pressure sensor connected to the fittings to measure pressure at a point between the pump and the aspiration catheter assembly.

A3. The apparatus of claim A1 wherein the conduit comprises polymer tubing and wherein the first automatic valve is a solenoid valve that fits around and engages the exterior of the polymer tubing.

A4. The apparatus of claim A2 wherein the order of components from the pump to the fittings is: the first automatic valve, the filter, the flow meter, and the first pressure sensor.

A5. The apparatus of claim A2 wherein the order of components from the pump to the fittings is: the filter, the flow meter, the first pressure sensor, and the automatic pump.

A6. The apparatus of claim A5 further comprising a second automatic valve between the filter and the pump.

A7. The apparatus of claim A6 further comprising a second pressure sensor between the pump and the second automatic valve.

A8. The apparatus of claim A1 wherein the controller comprises a processor with suitable memory and instructions, displays, and interfaces.

A9. The apparatus of claim A8 wherein the controller is programmed to execute any of the methods of claims 1-15.

B1. A method for aspirating a clot from a blood vessel using an aspiration catheter system, the method comprising:
applying pulsed aspiration with alternating aspiration on periods separated by aspiration off periods, wherein the aspiration off periods are no more than half as long as the aspiration on periods and wherein the aspiration on periods are from about 0.25 second to about 25 seconds.

B2. The method of claim B1 wherein the aspiration off periods are no more than about 75% of the aspiration on periods.

B3. The method of claim B1 wherein the aspiration off periods are from about 0.025 seconds to about 0.25 seconds.

B4. The method of claim B1 further comprising initially applying continuous aspiration and evaluating flow during application of continuous aspiration.

B5. The method of claim B4 wherein the pulsed aspiration is applied after measurement of a flow rate value of no more than a specified value.

B6. The method of claim B5 wherein the pulsed aspiration involves a set of one to 30 cycles of closing for aspiration off periods and opening for aspiration on periods an automatic valve and wherein after the last pulsed aspiration cycle the flow rate is measured to determine if a further set of aspiration pulse cycles should be performed based on flow rate measurements or alternatively whether continuous aspiration should be applied.

B7. The method of claim B1 wherein pressure is measured with a valve closed and wherein if the measured pressure greater than a threshold value the valve is opened and continuous aspiration is provided and if the measured pressure is below the threshold value, pulsed aspiration is continued.

B8. The method of claim B7 wherein during continuous aspiration, the flow rate is evaluated periodically and aspiration is terminated once the flow rate indicates that the clot likely has been cleared.

B9. The method of claim B7 wherein pulsed aspiration is resumed after a measurement of flow rate that falls below a threshold value.

B10. The method of claim B1 wherein the aspiration pulse is repeated for two to 25 times followed by a measurement of flow rate to determine if pulsed flow continues if the flow rate is below a threshold value or continuous flow continues if the flow rate is greater than the threshold value.

B11. The method of claim B1 wherein the aspiration catheter system comprises an aspiration catheter assembly comprising an aspiration catheter; fittings comprising a branched manifold with a first branch comprising a hemostatic valve and a second branch comprising a connector; a pump; a conduit connected to the pump and to the connector of the second branch; a flow meter connected to the fittings to measure flow to the pump; and a controller.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

What is claimed is:

1. A method for aspirating a clot from a blood vessel using an aspiration catheter system, the method comprising:
initiating calibrated continuous aspiration;
evaluating aspiration measurements; and
adjusting application of aspiration based on aspiration measurements.

2. The method of claim 1 wherein the aspiration catheter system comprises an aspiration catheter assembly comprising an aspiration catheter; fittings comprising a branched manifold with a first branch comprising a hemostatic valve and a second branch comprising a connector; a pump; a conduit connected to the pump and to the connector of the second branch; a flow meter connected to the fittings to measure flow to the pump; and a controller.

3. The method of claim 1 wherein the aspiration measurements comprise flow rate and pressure measurements.

4. The method of claim 3 wherein the pressure and flow rate measurements are used to estimate clot hardness by examining the flow rate relative to a maximum flow limit and the time dependence over a period of no more than about 2 seconds.

5. The method of claim 3 wherein if a flow rate reading is below a specified value, adjusting application of aspiration comprises initiating application of pulsed aspiration.

6. The method of claim 5 wherein an aspiration pulse involves closing an automatic valve for a first period of time and opening the valve for a second period of time.

7. The method of claim 5 wherein the pulsed aspiration involves a set of one to 30 cycles of closing for a first period of time and opening for a second period of time an automatic valve and wherein after the last cycle the flow rate is measured to determine if a further set of cycles should be performed based on flow rate measurements or alternatively whether continuous aspiration should be applied.

8. The method of claim 7 wherein the first period of time is less than the second period of time.

9. The method of claim 7 wherein during application of continuous aspiration, flow rate is periodically checked to determine if the clot has likely cleared the catheter and aspiration is stopped if the clot has cleared the catheter.

10. The method of claim 7 wherein the flow rate measurement comprises evaluating whether or not the flow rate is below a set value and/or whether the flow rate is increasing over time greater than a specified slope and wherein if the flow rate is below a specified value and/or the flow rate is not increasing at a sufficient rate then another set of pulsed aspiration cycles is performed.

11. The method of claim 1 wherein during application of continuous aspiration, flow rate is periodically checked to determine if the clot has likely cleared the catheter.

12. The method of claim 4 wherein if a clot is estimated to not be soft, adjusting application of aspiration comprises initiating pulsed aspiration at a frequency of at least about 20 hertz.

13. The method of claim 1 wherein aspiration measurements comprise a flow rate reading indicating flow rate through an open catheter within about 5%, and/or a pressure reading in the catheter within about 5% of the pump pressure with unrestrained flow that indicate clot clearance, and further comprising stopping aspiration once these measurements are received indicating clot clearance.

14. The method of claim 13 further comprising receiving a measurement of a value from a sensor in a filter indicating the presence of the clot in the filter and evaluating clot capture prior to stopping aspiration.

15. The method of claim 1 further comprising calibrating flow rate and/or pressure prior to delivering aspiration for clot removal, wherein calibrating comprises measuring flow rate and/or pressure with an open valve to a pump with the catheter inserted into to fluid reservoir and measuring flow rate and/or pressure with the valve closed.

16. The method of claim 1 wherein an evaluation of the clot is soft is based on the measurement of a pressure spike of at least about 10% relative to the pump pressure within bout 0.5 s of initiating aspiration.

17. An aspiration thrombectomy system comprising:
an aspiration catheter assembly comprising a suction lumen extending from a proximal end with a connector, to a distal opening;
fittings comprising a branched manifold with a first branch comprising a hemostatic valve and a second branch comprising a connector, wherein the fittings are in fluid communication with the suction lumen of the aspiration catheter;
a pump;
a conduit connected to the pump and to the connector of the second branch;
a flow meter connected to the conduit to measure flow rate to the pump;
a filter connected to the conduit to remove clots from the flow;
a first automatic valve configured to control flow between the fittings and the suction lumen; and
a controller connected to the flow meter and the automatic valve and wherein the controller controls the valve based on measurements received from the flow meter and pulses the valve based on measured flow values.

18. The apparatus of claim 17 further comprising a first pressure sensor connected to the fittings to measure pressure at a point between the pump and the aspiration catheter assembly.

19. A method for aspirating a clot from a blood vessel using an aspiration catheter system, the method comprising:
applying pulsed aspiration with alternating aspiration on periods separated by aspiration off periods, wherein the aspiration off periods are no more than half as long as the aspiration on periods and wherein the aspiration on periods are from about 0.25 second to about 25 seconds.

20. The method of claim 19 wherein the aspiration off periods are no more than about 75% of the aspiration on periods.

21. The method of claim 19 further comprising initially applying continuous aspiration and evaluating flow during application of continuous aspiration.

22. The method of claim 21 wherein the pulsed aspiration is applied after measurement of a flow rate value of no more than a specified value and wherein the pulsed aspiration involves a set of one to 30 cycles of closing for aspiration off periods and opening for aspiration on periods an automatic valve and wherein after the last pulsed aspiration cycle the flow rate is measured to determine if a further set of aspiration pulse cycles should be performed based on flow rate measurements or alternatively whether continuous aspiration should be applied.
